(19) United States
(12) Patent Application Publication
(10) Pub. No.: US 2023/0114654 A1
(43) Pub. Date: Apr. 13, 2023

(54) LIGHT-EMITTING DEVICE AND ELECTRONIC APPARATUS INCLUDING THE SAME

(71) Applicant: Samsung Display Co., Ltd., Yongin-si (KR)

(72) Inventors: Kunwook CHO, Yongin-si (KR); Kyungsik KIM, Yongin-si (KR); Yeongrong PARK, Yongin-si (KR); Jaeyong LEE, Yongin-si (KR); Hyeonmi LEE, Yongin-si (KR)

(21) Appl. No.: 17/880,593

(22) Filed: Aug. 3, 2022

(30) Foreign Application Priority Data
Aug. 4, 2021 (KR) 10-2021-0102655

(51) Int. Cl. H01L 51/00; C07D 498/06; C09K 11/06

(52) U.S. Cl. CPC H01L 51/0071; C07D 498/06; C09K 11/06; H01L 51/0052; H01L 51/0073; H01L 51/0058; H01L 51/5056

(57) ABSTRACT

A light-emitting device includes: a first electrode; a second electrode facing the first electrode; and an interlayer located between the first electrode and the second electrode and including an emission layer, wherein the first electrode includes an inorganic material, the inorganic material includes a metal oxide having, as a main component, at least one metal selected from W, Mo, Ga, Ni, Cu, Zn, and Ti, the interlayer further includes a hole transport region located between the first electrode and the emission layer, the hole transport region includes a hole transport layer, and the hole transport layer includes at least one condensed cyclic compound represented by Formula 1:

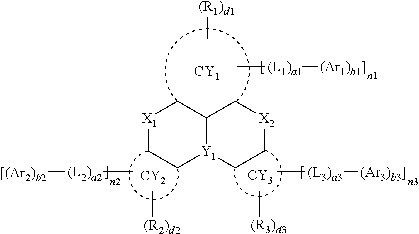

### CROSS-REFERENCE TO RELATED APPLICATION

This application claims from and the benefit of Korean Patent Application No. 10-2021-0102655, filed on Aug. 4, 2021, which is hereby incorporated by reference for all purposes as if fully set forth herein.

### BACKGROUND

### Field

Embodiments of the invention relate generally to a light-emitting device and an electronic apparatus including the same.

### Discussion of the Background

From among light-emitting devices, self-emissive devices have wide viewing angles, high contrast ratios, short response times, and excellent characteristics in terms of luminance, driving voltage, and response speed.

In a light-emitting device, a first electrode is located on a substrate, and a hole transport region, an emission layer, an electron transport region, and a second electrode are sequentially located on the first electrode. Holes provided from the first electrode move toward the emission layer through the hole transport region, and electrons provided from the second electrode move toward the emission layer through the electron transport region. Carriers, such as holes and electrons, recombine in the emission layer to produce excitons. These excitons transition from an excited state to a ground state, thereby generating light.

The above information disclosed in this Background section is only for understanding of the background of the inventive concepts, and, therefore, it may contain information that does not constitute prior art.

### SUMMARY

Devices constructed according to illustrative implementations of the invention are capable of at least one of high device stability, low progressive driving voltage, high efficiency, and a long lifespan.

Inventive concepts consistent with one or more embodiments provide for a light-emitting device having high device stability, low progressive driving voltage, high efficiency, and a long lifespan.

Additional features of the inventive concepts will be set forth in the description that follows, and in part will be apparent from the description, or may be learned by practice of the inventive concepts.

According to one or more embodiments, a light-emitting device includes a first electrode, a second electrode facing the first electrode, and an interlayer between the first electrode and the second electrode and including an emission layer, wherein the first electrode includes an inorganic material, and the inorganic material includes at least one metal selected from W, Mo, Ga, Ni, Cu, Zn, and Ti. The interlayer includes a hole transport region between the first electrode and the emission layer, the hole transport region includes a hole transport layer, and the hole transport layer includes at least one condensed cyclic compound represented by Formula 1.

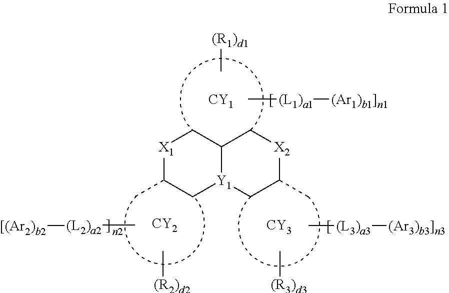

In Formula 1,
CY₁ to CY₃ may each independently be a C₅-C₆₀ carbocyclic group or a C₁-C₆₀ heterocyclic group,
X₁ may be C(R₄)(R₅), N(R₄), O, or S,
X₂ may be C(R₆)(R₇), N(R₆), O, or S,
Y₁ may be N, B, P, or P(═O),
L₁ to L₃ may each independently be a single bond, a C₅-C₆₀ carbocyclic group unsubstituted or substituted with at least one R₁₀ₐ, or a C₁-C₆₀ heterocyclic group unsubstituted or substituted with at least one R₁₀ₐ,
a1 to a3 may each independently be an integer from 1 to 5,
Ar₁ to Ar₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
b1 to b3 may each independently be an integer from 1 to 12,
n1 to n3 may each independently be an integer from 0 to 5,
the sum of n1, n2, and n3 may be 1 or more,
R₁ to R₇ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
d1 to d3 may each independently be an integer from 1 to 12,
R₁₀ₐ may be deuterium, —F, —Cl, —Br, —I, a hydroxyl group, a cyano group, or a nitro group, a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof, a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof, or —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, or any combination thereof.

According to one or more embodiments, an electronic apparatus includes the light-emitting device.

It is to be understood that both the foregoing general description and the following detailed description are illustrative and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate illustrative embodiments of the invention, and together with the description serve to explain the inventive concepts.

FIG. 1 shows a schematic cross-sectional view of a light-emitting device according to an embodiment that is constructed according to principles of the invention.

FIG. 2 shows a schematic cross-sectional view of an electronic apparatus according to an embodiment.

FIG. 3 shows a schematic cross-sectional view of an electronic apparatus according to an embodiment.

FIG. 4 is a graph showing the variation in driving voltage (ΔV) over time (hr) for light-emitting devices of Examples 1 to 5 and Comparative Examples 1 to 5 under conditions of room temperature and luminance of 420 nits.

### DETAILED DESCRIPTION

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing illustrative features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of idealized embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

The term “interlayer” as used herein refers to a single layer and/or all layers between a first electrode and a second electrode of a light-emitting device.

The expression “(an interlayer and/or a capping layer) includes a compound represented by Formula 1” as used herein may include a case in which “(an interlayer and/or a capping layer) includes identical compounds represented by Formula 1” and a case in which “(an interlayer and/or a capping layer) includes two or more different compounds represented by Formula 1”.

In the embodiments described hereinbelow, a highest occupied molecular orbital (HOMO) energy level and work function of materials will be described later, but embodiments as described herein are not limited thereto.

A HOMO energy level of materials is measured by cyclic voltammetry, and a cyclic voltammetry apparatus used herein is ZIVE SP2 available from Wonatech. Respective sample solutions and electrolytic solutions used herein are as follows. Ferrocene is used as a reference material, and (Bu)₄NPF₆ is used as an electrolyte:

sample solution of target compound: 5×10⁻³ M dichloromethane solution;

ferrocene sample solution: 5×10⁻³ M dichloromethane solution; and

(Bu)₄NPF₆ electrolytic solution: 0.1 M acetonitrile solution.

An E_{we}-I relationship graph of target compounds and a reference material is obtained first. Then, tangent lines are drawn at every point where the current rapidly increases in the graph, and the voltage at the points where the tangent lines meet the x-axis is recorded. A HOMO energy level of ferrocene is set to −4.8 eV, and a HOMO energy level of target compounds is calculated.

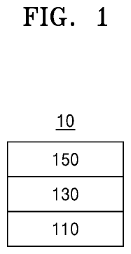

Work function of materials is evaluated as follows: respective materials are spin-coated on an ITO substrate to form a 50-nm thin film, followed by heat treatment at 200° C. for 5 minutes on a hot plate in the air. An equipment used for this evaluation is a ultraviolet photoelectron spectroscopy (UPS) equipment.

[Description of FIG. 1]

FIG. 1 is a schematic cross-sectional view of a light-emitting device 10 according to an embodiment that is constructed according to principles of the invention. The light-emitting device 10 includes a first electrode 110, an interlayer 130, and a second electrode 150.

Hereinafter, the structure of the light-emitting device 10 according to an embodiment and a method of manufacturing the light-emitting device 10 will be described with reference to FIG. 1.

[First Electrode 110]

In FIG. 1, a substrate may be additionally located under the first electrode 110 or on the second electrode 150. In an embodiment, as the substrate, a glass substrate or a plastic substrate may be used. In one or more embodiments, the substrate may be a flexible substrate, and may include plastics with excellent heat resistance and durability, such as polyimide, polyethylene terephthalate (PET), polycarbonate, polyethylene napthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a semi-transmissive electrode, or a transmissive electrode.

The first electrode 110 may include an inorganic material. The inorganic material may include a metal oxide having at least one metal selected from W, Mo, Ga, Ni, Cu, Zn, and Ti. For example, the inorganic material may include a metal oxide having, as a main component, at least one metal selected from W, Mo, Ga, Ni, Cu, Zn, and Ti.

For example, the inorganic material may include a metal oxide having, as a main component, at least one metal selected from W, Mo, Ga, Ni, and Cu.

In an embodiment, the inorganic material may include WOₓ, MoOₓ, GaOₓ, NiO_{y}, CuO_{y}, or any combination thereof (wherein x is a real number satisfying 2.5≤x≤3.0, and y is a real number satisfying 0.5≤y≤2.0).

When the first electrode 110 includes the inorganic material, an absolute value of the work function of the first electrode 110 may increase. For example, the first electrode 110 further including the inorganic material may have a larger absolute value of the work function than the first electrode 110 including only a conductive oxide material as a material for forming the first electrode 110.

When ITO which is an existing anode material for a light-emitting device in the art is used, the absolute value of the work function of the first material 110 may be about 4.8 eV which is not considered deep. That is, the energy level between an anode and a cathode is not significantly large, so that a hole transport layer material is limited in terms of improving hole-transporting characteristics and device stability. Consequently, the light-emitting device has problems of decreased luminescence efficiency and a short lifespan.

In the light-emitting device 10 according to an embodiment, the absolute value of the work function of the first electrode 110 may increase by applying the inorganic material to the first electrode 110, thereby improving hole injection efficiency and device stability.

In an embodiment, the absolute value of the work function of the first electrode 110 may be 5.3 eV or more.

In an embodiment, the first electrode 110 may have a multi-layered structure including a plurality of layers.

For example, the first electrode 110 may have a structure consisting of three or more layers.

In an embodiment, the first electrode 110 may include: a first layer including a first material; a second layer including a second material; and a third layer including the inorganic material.

The third layer may be located between the first layer and the interlayer.

The second layer may be located between the first layer and the third layer.

For example, the second layer may be located on the first layer, the third layer may be located on the second layer, and the interlayer may be located on the third layer. That is, the first layer, the second layer, the third layer, and the interlayer may be located in this stated order.

The first material and the second material may be different from each other.

The second material and the inorganic material may be different from each other.

In an embodiment, the first layer may be in direct contact with the second layer.

In an embodiment, the second layer may be in direct contact with the third layer.

In an embodiment, the third layer may be in direct contact with the interlayer.

In an embodiment, the first material and the inorganic material may be different from each other.

In an embodiment, the first material may include a conductive oxide material.

In the light-emitting device 10 according to an embodiment, the first electrode 110 may include the first layer including the conductive oxide material, and in this regard, the first electrode 110 may serve to prevent contact between glass and the second layer including the second material. Accordingly, an issue of decreased reflectance by aggregation caused by contact between glass and the second material including a metal material or a metal alloy metal may be prevented, thereby improving efficiency of the light-emitting device 10. In addition, when the first material has conductivity, electron transfer through contact with a source electrode and a drain electrode in the panel structure of the light-emitting device 10 may be facilitated.

For example, the first material may include indium tin oxide (ITO), indium zinc oxide (IZO), tin oxide (SnO₂), zinc oxide (ZnO), or any combination thereof.

In an embodiment, the first layer may consist of the first material.

In an embodiment, the second material may include a metal material or a metal alloy material.

In the light-emitting device 10 according to an embodiment, when the first electrode 110 includes the second layer including the metal material or the metal alloy material and light emitted from an emission layer reaches a cathode, the light is reflected as it is, thereby improving the light characteristics of the light-emitting device 10 through the effect of overlapping with other light sources.

For example, the second material may include magnesium (Mg), silver (Ag), aluminum (Al), aluminum-lithium (Al—Li), calcium (Ca), magnesium-indium (Mg—In), magnesium-silver (Mg—Ag), AlNiLa, AlNd, AlNiGeLa, AlCoGeLa, or any combination thereof.

The term “AlNiLa” as used herein refers to an aluminum-nickel-lanthanum alloy, and for example, an amount of nickel may be in a range of about 1 atom % to about 3 atom %, and an amount of lanthanum may be in a range of about 0.1 atom % to about 0.5 atom %.

The term “AlNd” as used herein refers to an aluminum-neodymium alloy, and for example, an amount of neodymium may be in a range of about 1 atom % to about 3 atom %.

The term “AlNiGeLa” as used herein refers to an aluminum-nickel-germanium-lanthanum alloy, and for example, an amount of nickel may be in a range of about 1 atom % to about 3 atom %, an amount of germanium may be in a range of about 1 atom % to about 3 atom %, and an amount of lanthanum may be in a range of about 0.01 atom % to about 0.2 atom %.

The term “AlCoGeLa” as used herein refers to an aluminum-cobalt-germanium-lanthanum alloy, and for example, an amount of cobalt may be in a range of about 1 atom % to about 3 atom %, an amount of germanium may be in a range of about 1 atom % to about 3 atom %, and an amount of lanthanum may be in a range of about 0.01 atom % to about 0.2 atom %.

In an embodiment, the second layer may consist of the second material.

In an embodiment, the third layer may consist of the inorganic material.

[Interlayer 130]

The interlayer 130 may be located on the first electrode 110. The interlayer 130 may include an emission layer. The interlayer 130 may further include a hole transport region between the first electrode 110 and the emission layer, and an electron transport region between the emission layer and the second electrode 150.

The interlayer 130 may further include, in addition to various organic materials, a metal-containing compound, such as an organometallic compound, an inorganic material, such as a quantum dot, and the like.

In an embodiment, the interlayer 130 may include: i) two or more light-emitting units sequentially stacked between the first electrode 110 and the second electrode 150; and ii) a charge generation layer located between the two or more light-emitting units. When the interlayer 130 includes the two or more light-emitting units and the charge generation layer as described above, the light-emitting device 10 may be a tandem light-emitting device.

[Hole Transport Region in Interlayer 130]

The hole transport region may include a hole transport layer.

The hole transport region may have i) a single-layered structure consisting of a single layer consisting of a single material, ii) a single-layered structure consisting of a single layer consisting of a plurality of different materials, or iii) a multi-layered structure including a plurality of layers including different materials.

The hole transport region may include a hole injection layer, an emission auxiliary layer, an electron blocking layer, or any combination thereof.

For example, the hole transport region may have: a single-layered structure consisting of a hole transport layer; or a multi-layered structure, such as a hole transport layer/emission auxiliary layer structure or a hole transport layer/electron blocking layer structure, wherein constituting layers for each structure are sequentially stacked in the stated order on the first electrode 110.

[Hole Transport Layer]

The hole transport layer may include at least one condensed cyclic compound represented by Formula 1:

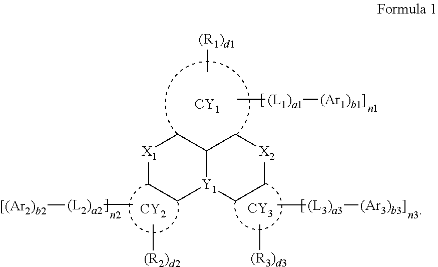

In Formula 1,
CY₁ to CY₃ may each independently be a C₅-C₆₀ carbocyclic group or a C₁-C₆₀ heterocyclic group.

In an embodiment, CY₁ to CY₃ may each independently be a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluorene-9-one group, an azadibenzothiophene 5,5-dioxide group, an indolocarbazole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an iso-oxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group.

X₁ may be C(R₄)(R₅), N(R₄), O, or S.

X₂ may be C(R₆)(R₇), N(R₆), O, or S.

For example, X₁ may be O, and X₂ may be O.

Y₁ may be N, B, P, or P(═O).

For example, Y₁ may be N.

L₁ to L₃ may each independently be a single bond, a C₅-C₆₀ carbocyclic group unsubstituted or substituted with at least one R₁₀ₐ, or a C₁-C₆₀ heterocyclic group unsubstituted or substituted with at least one R₁₀ₐ.

In an embodiment, L₁ to L₃ may each independently be a single bond; or a group represented by one of Formulas 10-1 to 10-40:

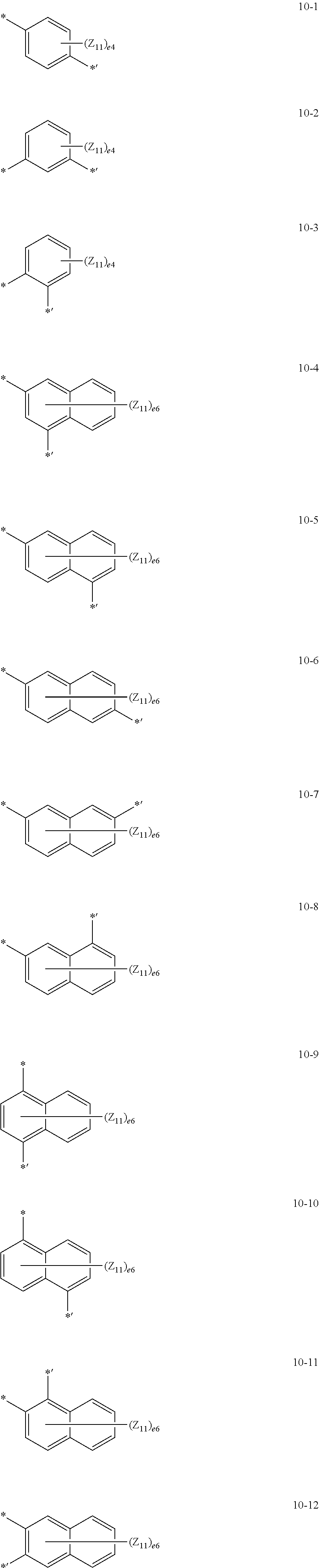

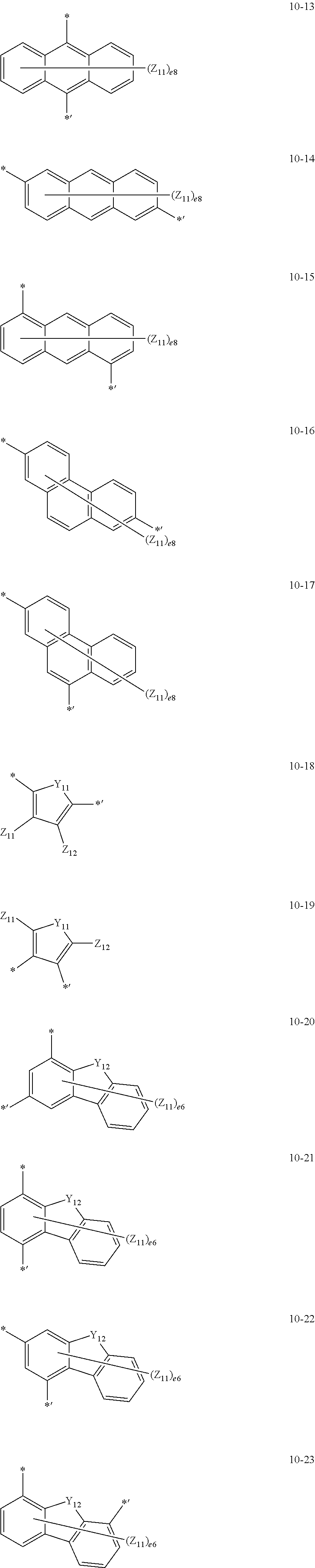

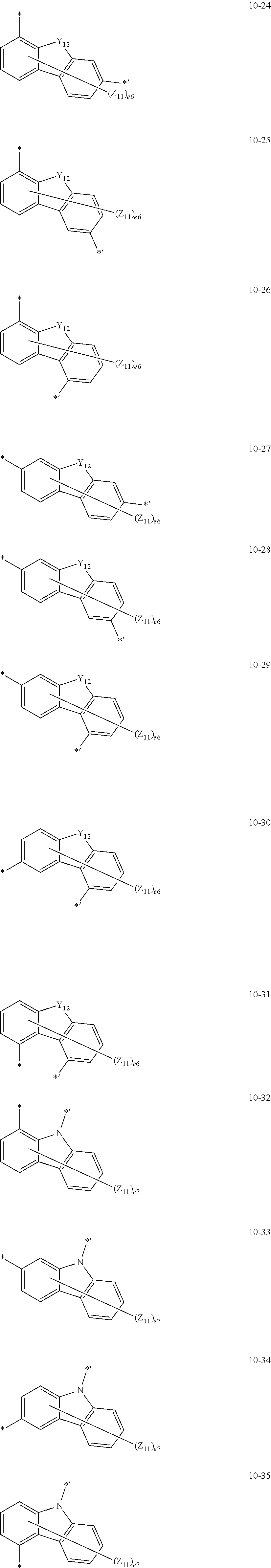

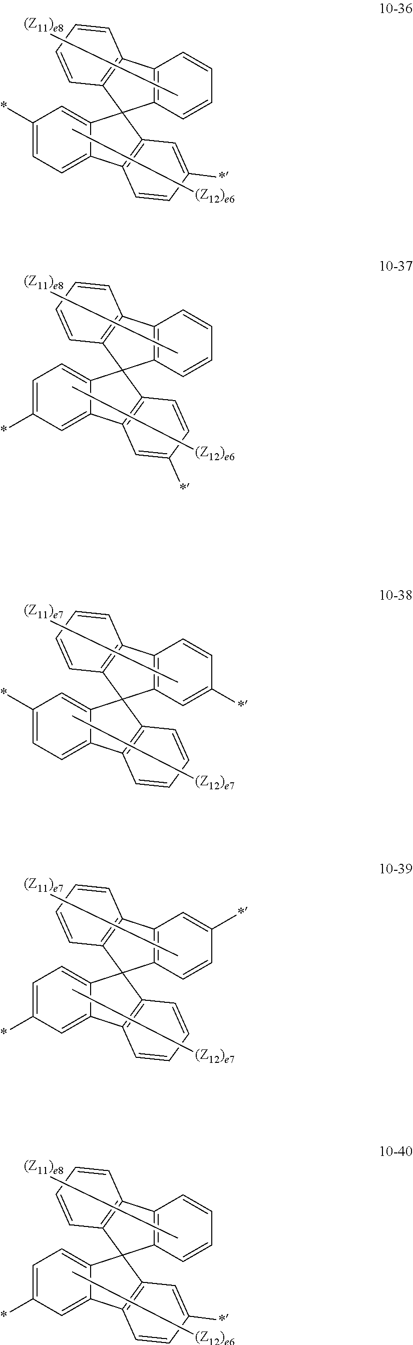

In Formulas 10-1 to 10-40,
Y₁₁ may be O or S,
Y₁₂ may be O, S, N(Z₁₃), or C(Z₁₃)(Z₁₄),
Z₁₁ to Z₁₄ may each independently be hydrogen, deuterium, —F, —Cl, —Br, —I, a hydroxyl group, a cyano group, a nitro group, an amidino group, a hydrazino group, a hydrazono group, —CF₃, —CF₂H, —CFH₂, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyridinyl group, a pyrimidinyl group, a pyrazinyl group, a triazinyl group, a quinolinyl group, an isoquinolinyl group, a benzoquinolinyl group, a naphthyridinyl group, a quinoxalinyl group, a quinazolinyl group, a phenanthridinyl group, an acridinyl group, a phenanthrolinyl group, a phenazinyl group, a carbazolyl group, a dibenzofuranyl group, a dibenzothiophenyl group, a dibenzosilolyl group, —Si(Q₃₁)(Q₃₂)(Q₃₃), —N(Q₃₁)(Q₃₂), or —B(Q₃₁)(Q₃₂),
e4 may be an integer from 1 to 4,
e6 may be an integer from 1 to 6,
e7 may be an integer from 1 to 7,
e8 may be an integer from 1 to 8,
Q₃₁ to Q₃₃ may each independently be a C₁-C₁₀ alkyl group, a C₁-C₁₀ alkoxy group, a phenyl group, a biphenyl group, a terphenyl group, or a naphthyl group, and
* and *′ each indicate a binding site to a neighboring atom, and
a1 to a3 may each independently be an integer from 1 to 5.

In an embodiment, Ar₁ to Ar₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ.

In one or more embodiments, Ar₁ to Ar₃ may each independently be a benzene group, a naphthalene group, an anthracene group, a phenanthrene group, a triphenylene group, a pyrene group, a chrysene group, a cyclopentadiene group, a 9,9′-spirobifluorene group, a spiro[cyclohexane-1,9′-fluorene] group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophenegroup, a dibenzofuran group, a dibenzothiophene 5-oxide group, a 9H-fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluoren-9-one group, an azadibenzothiophene 5,5-dioxide group, an indolocarbazole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isooxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group, each unsubstituted or substituted with at least one R₁₀ₐ, and

R₁₀ₐ may be the same as described herein.

In one or more embodiments, Ar₁ to Ar₃ may each independently represented by one of Formulas 2-1 to 2-36:

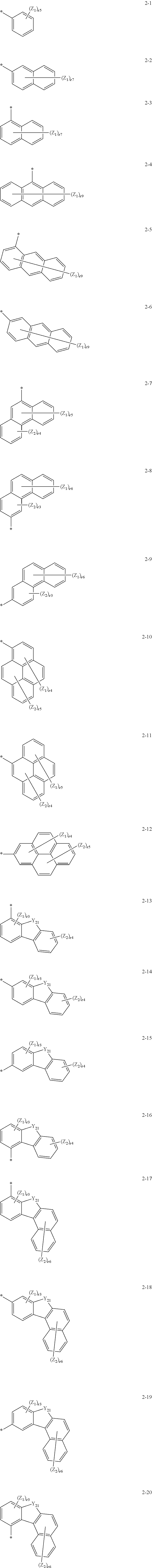

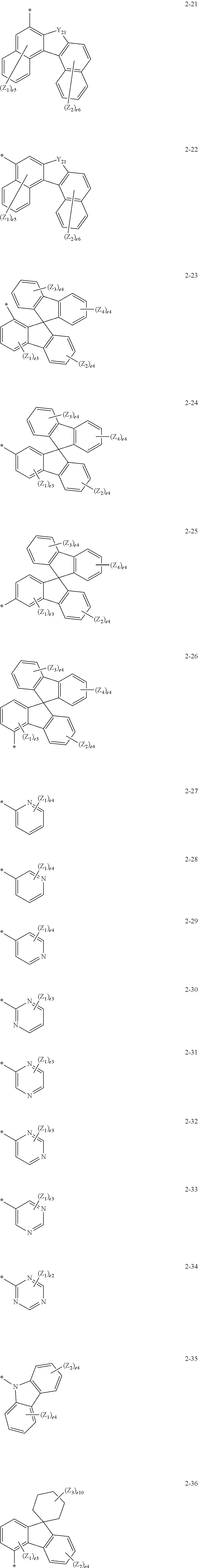

In Formulas 2-1 to 2-36

Y₂₁ may be O, S, N(Z₅), C(Z₅)(Z₆), or Si(Z₅)(Z₆),

Z₁ to Z₆ may each independently be the same as described in connection with R₁,

e2 may be 1 or 2,

e3 may be an integer from 1 to 3,

e4 may be an integer from 1 to 4,

e5 may be an integer from 1 to 5,

e6 may be an integer from 1 to 6,

e7 may be an integer from 1 to 7,

e9 may be an integer from 1 to 9,

e10 may be an integer from 1 to 10, and

* indicates a binding site to a neighboring atom.

In an embodiment, Z₁ to Z₆ may each independently be hydrogen, deuterium, —F, —Cl, —Br, —I, a hydroxyl group, a cyano group, a nitro group, an amidino group, a hydrazino group, a hydrazono group, —CF₃, —CF₂H, —CFH₂, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyridinyl group, a pyrimidinyl group, a pyrazinyl group, a triazinyl group, a quinolinyl group, an isoquinolinyl group, a benzoquinolinyl group, a naphthyridinyl group, a quinoxalinyl group, a quinazolinyl group, a phenanthridinyl group, an acridinyl group, a phenanthrolinyl group, a phenazinyl group, a carbazolyl group, a dibenzofuranyl group, a dibenzothiophenyl group, a dibenzosilolyl group, —Si(Q₃₁)(Q₃₂)(Q₃₃), —N(Q₃₁)(Q₃₂), or —B(Q₃₁)(Q₃₂), and
Q₃₁ to Q₃₃ may each independently be a C₁-C₁₀ alkyl group, a C₁-C₁₀ alkoxy group, a phenyl group, a biphenyl group, a terphenyl group, or a naphthyl group.

b1 to b3 may each independently be an integer from 1 to 12.

n1 to n3 may each independently be an integer from 0 to 5.

The sum of n1, n2, and n3 may be 1 or more.

For example, n1 may be 1, n2 may be 0, and n3 may be 0;

n1 may be 0, n2 may be 1, and n3 may be 0;

n1 may be 0, n2 may be 0, and n3 may be 1;

n1 may be 1, n2 may be 1, and n3 may be 0;

n1 may be 1, n2 may be 0, and n3 may be 1;

n1 may be 0, n2 may be 1, and n3 may be 1; or

n1 may be 1, n2 may be 1, and n3 may be 1.

R₁ to R₇ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

d1 to d3 may each independently be an integer from 1 to 12.

R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof; a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, or any combination thereof.

In an embodiment, the condensed cyclic compound represented by Formula 1 may be represented by one of Formulas 1-1 to 1-5:

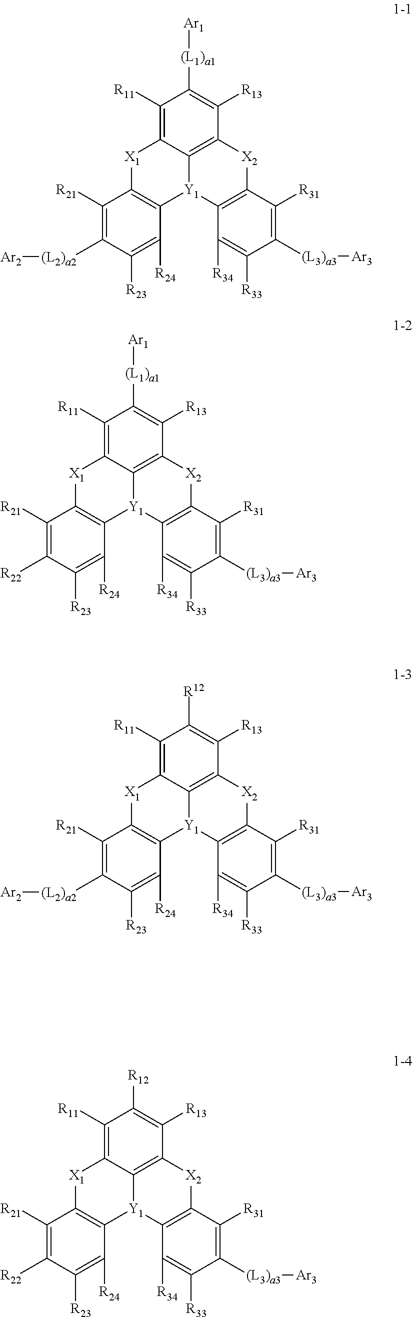

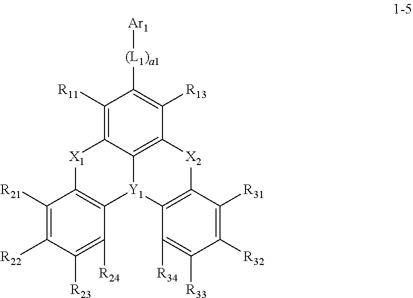

In Formulas 1-1 to 1-5,
X₁, X₂, Y₁, L₁ to L₃, a1 to a3, and Ar₁ to Ar₃ may respectively be the same as described herein,
R₁₁ to R₁₃ may respectively be the same as described in connection with R₁,
R₂₁ to R₂₄ may respectively be the same as described in connection with R₂, and
R₃₁ to R₃₄ may respectively be the same as described in connection with R₃.

In an embodiment, the condensed cyclic compound represented by Formula 1 may be one of Compounds 1 to 140, but embodiments as described herein are not limited thereto:

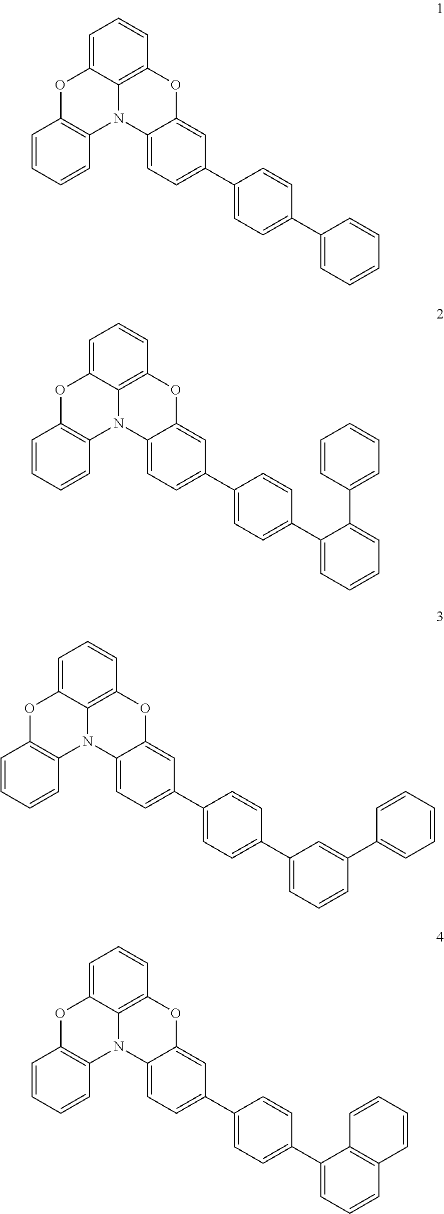 [SMILES: c1ccc(-c2ccc(-c3ccc4c(c3)Oc3cccc5c3N4c3ccccc3O5)cc2)cc1 ; c1ccc(-c2ccccc2-c2ccc(-c3ccc4c(c3)Oc3cccc5c3N4c3ccccc3O5)cc2)cc1 ; c1ccc(-c2cccc(-c3ccc(-c4ccc5c(c4)Oc4cccc6c4N5c4ccccc4O6)cc3)c2)cc1 ; c1ccc2c(c1)Oc1cccc3c1N2c1ccc(-c2ccc(-c4cccc5ccccc45)cc2)cc1O3]

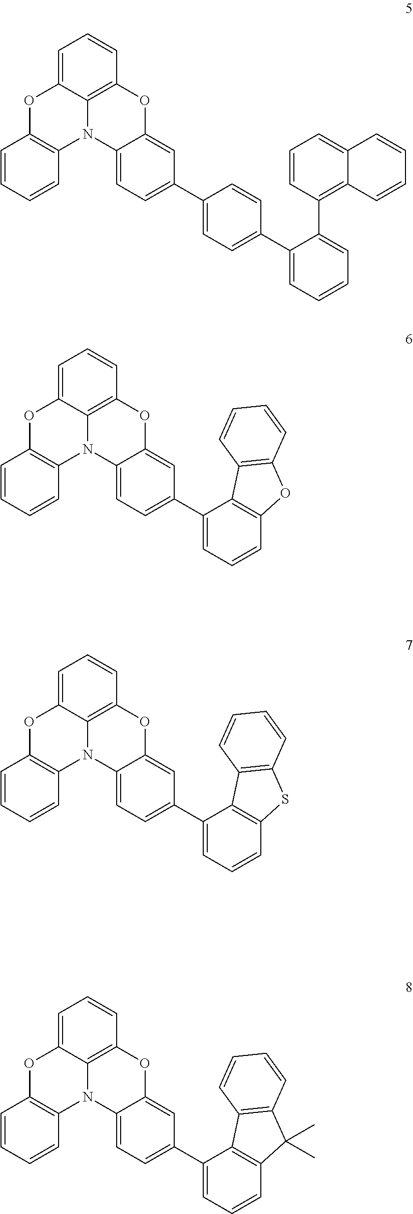 [SMILES: c1ccc2c(c1)Oc1cccc3c1N2c1ccc(-c2ccc(-c4ccccc4-c4cccc5ccccc45)cc2)cc1O3 ; c1ccc2c(c1)Oc1cccc3c1N2c1ccc(-c2cccc4oc5ccccc5c24)cc1O3 ; c1ccc2c(c1)Oc1cccc3c1N2c1ccc(-c2cccc4sc5ccccc5c24)cc1O3 ; CC1(C)c2ccccc2-c2c(-c3ccc4c(c3)Oc3cccc5c3N4c3ccccc3O5)cccc21]

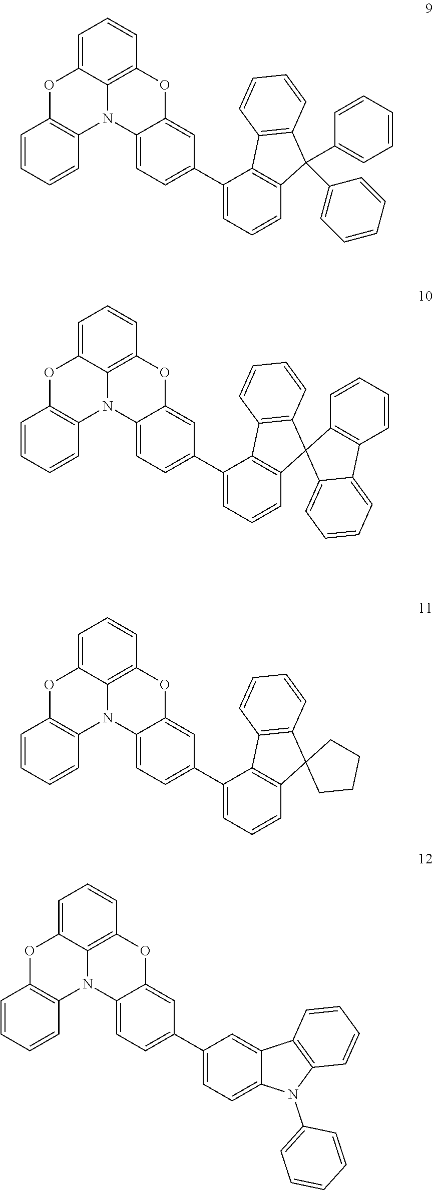

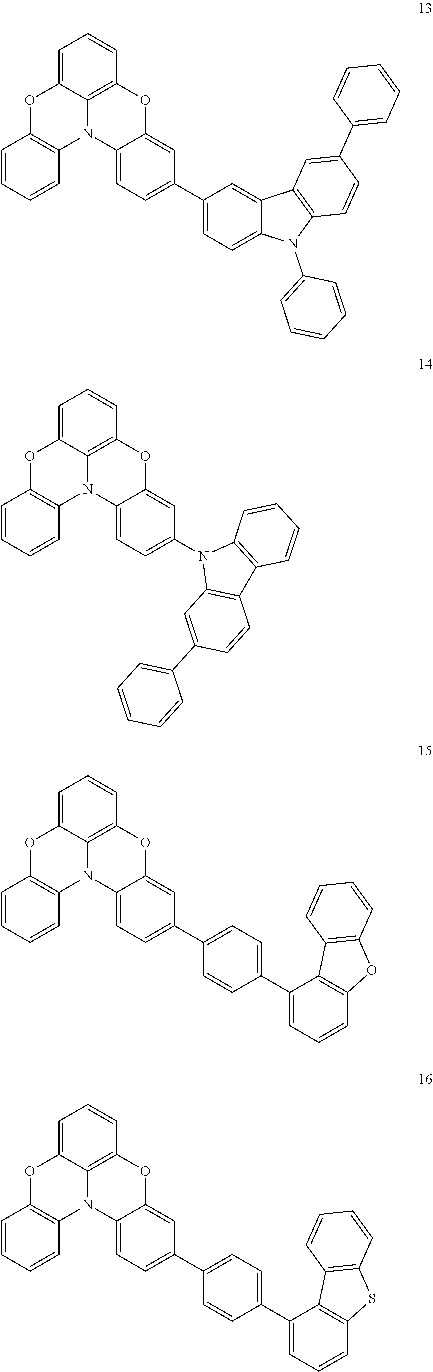

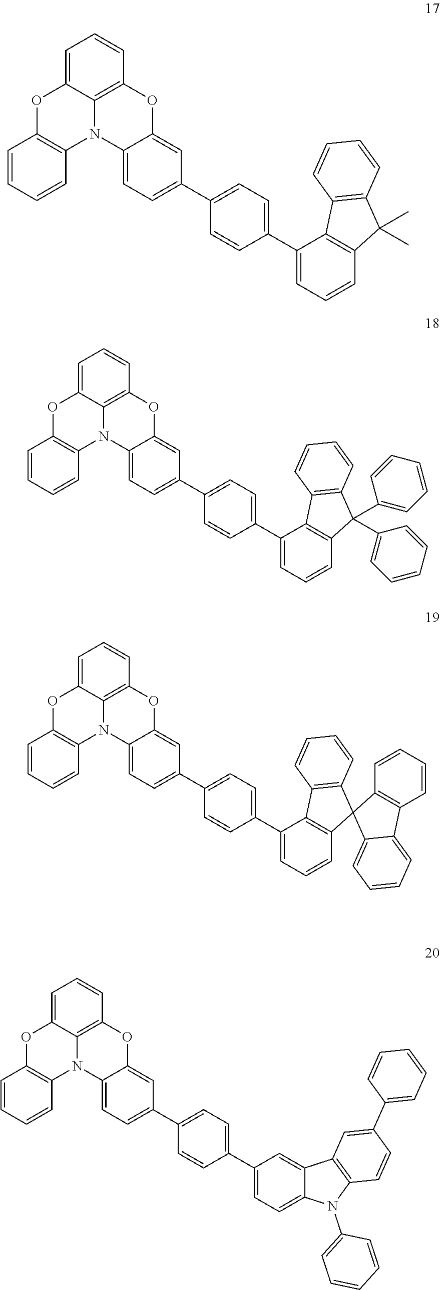

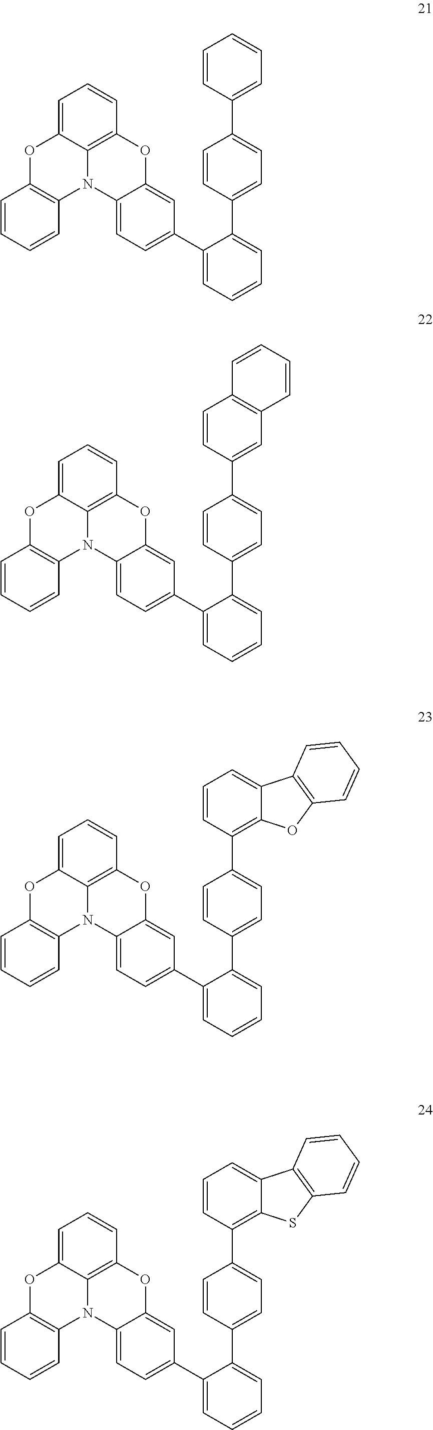

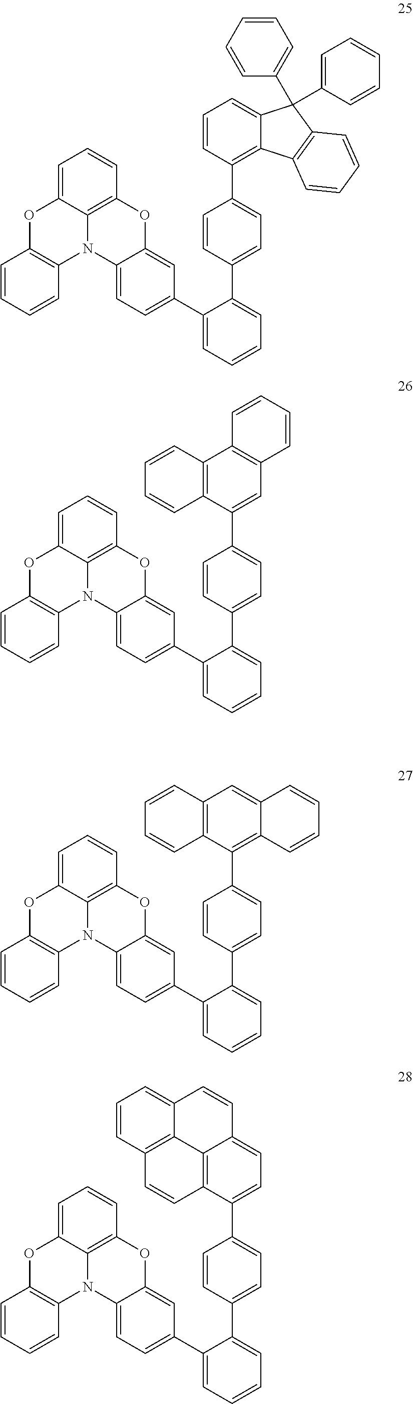

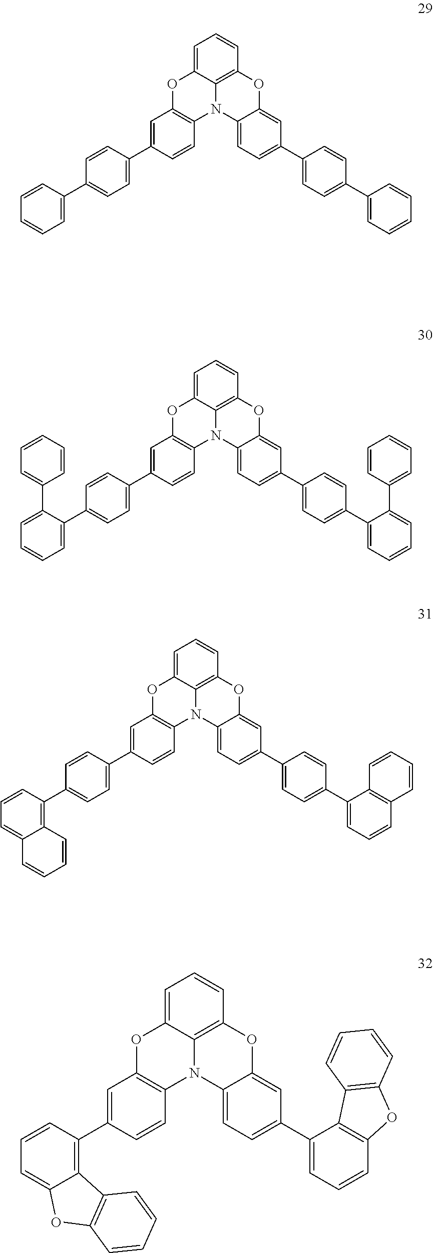 [SMILES: c1ccc(-c2ccc(-c3ccc4c(c3)Oc3cccc5c3N4c3ccc(-c4ccc(-c6ccccc6)cc4)cc3O5)cc2)cc1 ; c1ccc(-c2ccccc2-c2ccc(-c3ccc4c(c3)Oc3cccc5c3N4c3ccc(-c4ccc(-c6ccccc6-c6ccccc6)cc4)cc3O5)cc2)cc1 ; c1cc2c3c(c1)Oc1cc(-c4ccc(-c5cccc6ccccc56)cc4)ccc1N3c1ccc(-c3ccc(-c4cccc5ccccc45)cc3)cc1O2 ; c1cc2c3c(c1)Oc1cc(-c4cccc5oc6ccccc6c45)ccc1N3c1ccc(-c3cccc4oc5ccccc5c34)cc1O2]

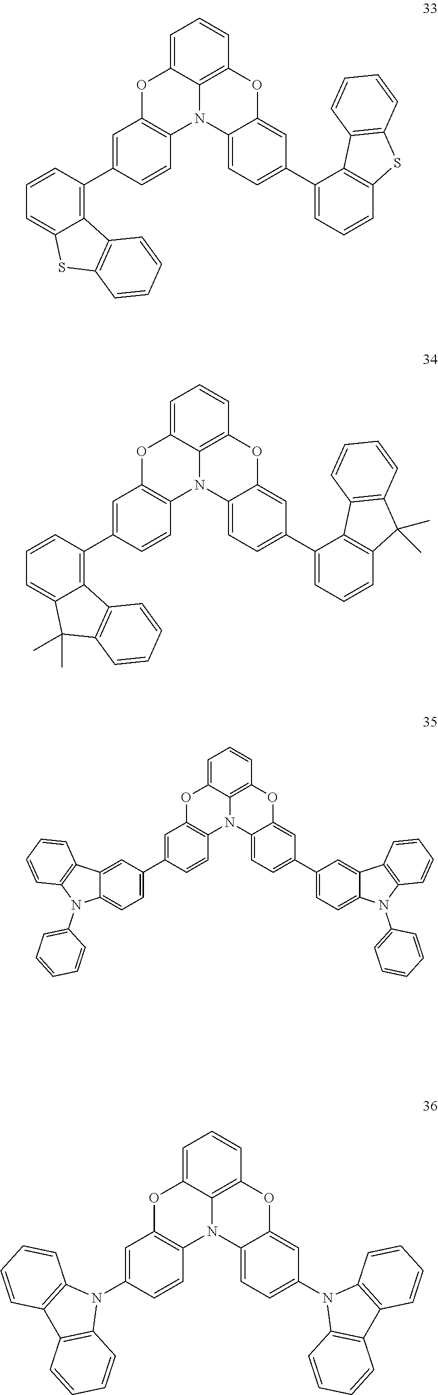 [SMILES: c1cc2c3c(c1)Oc1cc(-c4cccc5sc6ccccc6c45)ccc1N3c1ccc(-c3cccc4sc5ccccc5c34)cc1O2 ; CC1(C)c2ccccc2-c2c(-c3ccc4c(c3)Oc3cccc5c3N4c3ccc(-c4cccc6c4-c4ccccc4C6(C)C)cc3O5)cccc21 ; c1ccc(-n2c3ccccc3c3cc(-c4ccc5c(c4)Oc4cccc6c4N5c4ccc(-c5ccc7c(c5)c5ccccc5n7-c5ccccc5)cc4O6)ccc32)cc1 ; c1cc2c3c(c1)Oc1cc(-n4c5ccccc5c5ccccc54)ccc1N3c1ccc(-n3c4ccccc4c4ccccc43)cc1O2]

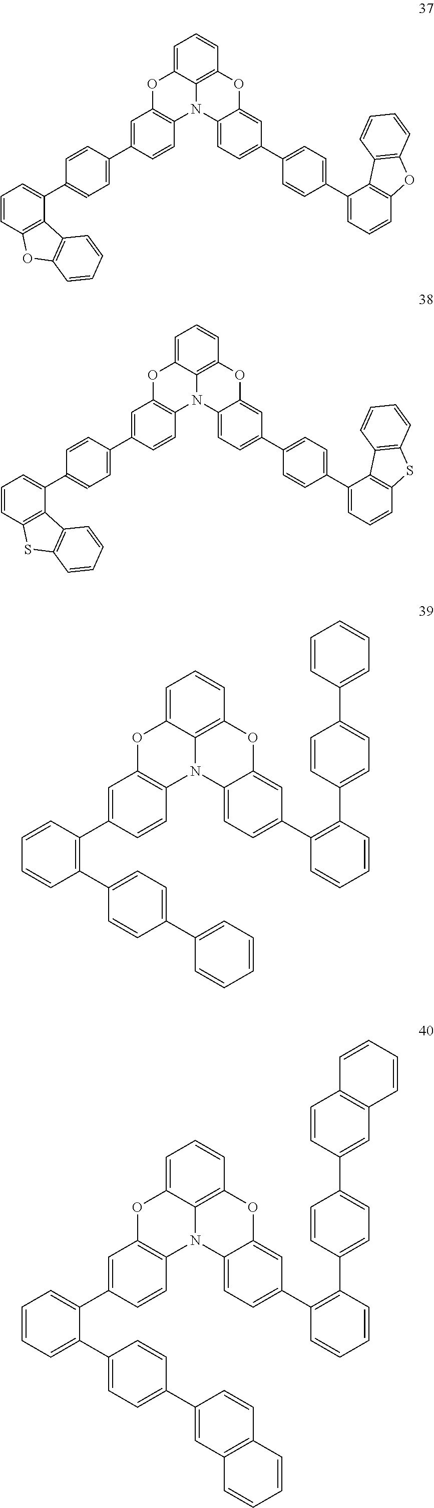 [SMILES: c1cc2c3c(c1)Oc1cc(-c4ccc(-c5cccc6oc7ccccc7c56)cc4)ccc1N3c1ccc(-c3ccc(-c4cccc5oc6ccccc6c45)cc3)cc1O2 ; c1cc2c3c(c1)Oc1cc(-c4ccc(-c5cccc6sc7ccccc7c56)cc4)ccc1N3c1ccc(-c3ccc(-c4cccc5sc6ccccc6c45)cc3)cc1O2 ; c1ccc(-c2ccc(-c3ccccc3-c3ccc4c(c3)Oc3cccc5c3N4c3ccc(-c4ccccc4-c4ccc(-c6ccccc6)cc4)cc3O5)cc2)cc1 ; c1cc2c3c(c1)Oc1cc(-c4ccccc4-c4ccc(-c5ccc6ccccc6c5)cc4)ccc1N3c1ccc(-c3ccccc3-c3ccc(-c4ccc5ccccc5c4)cc3)cc1O2]

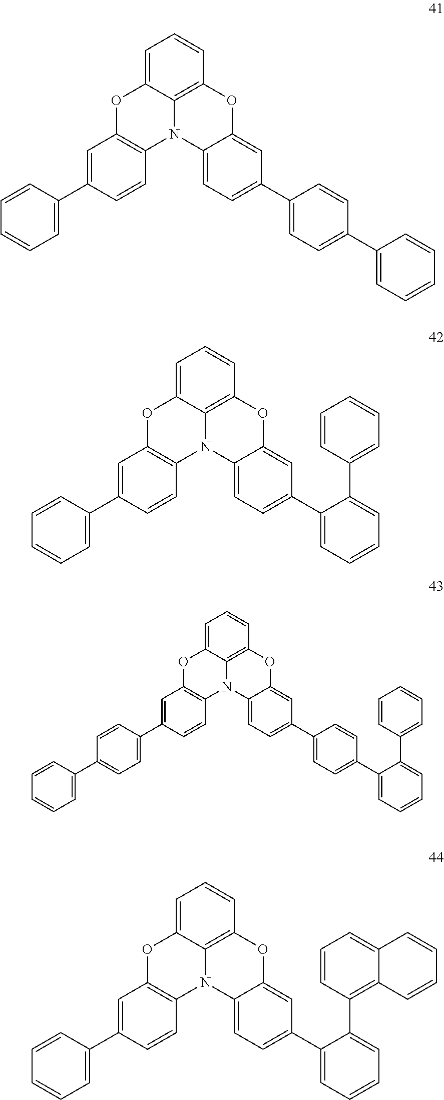 [SMILES: c1ccc(-c2ccc(-c3ccc4c(c3)Oc3cccc5c3N4c3ccc(-c4ccccc4)cc3O5)cc2)cc1 ; c1ccc(-c2ccc3c(c2)Oc2cccc4c2N3c2ccc(-c3ccccc3-c3ccccc3)cc2O4)cc1 ; c1ccc(-c2ccc(-c3ccc4c(c3)Oc3cccc5c3N4c3ccc(-c4ccc(-c6ccccc6-c6ccccc6)cc4)cc3O5)cc2)cc1 ; c1ccc(-c2ccc3c(c2)Oc2cccc4c2N3c2ccc(-c3ccccc3-c3cccc5ccccc35)cc2O4)cc1]

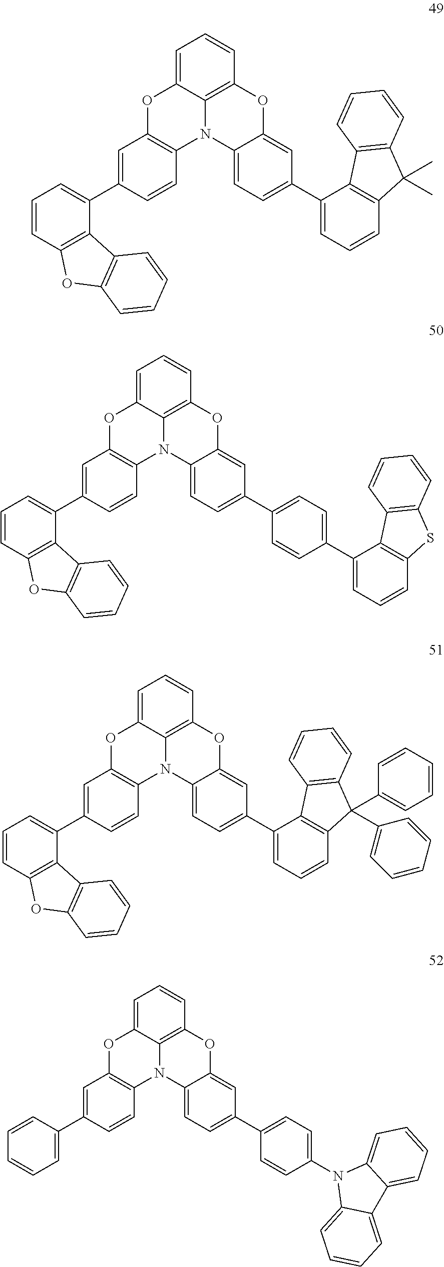 [SMILES: CC1(C)c2ccccc2-c2c(-c3ccc4c(c3)Oc3cccc5c3N4c3ccc(-c4cccc6oc7ccccc7c46)cc3O5)cccc21 ; c1cc2c3c(c1)Oc1cc(-c4cccc5oc6ccccc6c45)ccc1N3c1ccc(-c3ccc(-c4cccc5sc6ccccc6c45)cc3)cc1O2 ; c1ccc(C2(c3ccccc3)c3ccccc3-c3c(-c4ccc5c(c4)Oc4cccc6c4N5c4ccc(-c5cccc7oc8ccccc8c57)cc4O6)cccc32)cc1 ; c1ccc(-c2ccc3c(c2)Oc2cccc4c2N3c2ccc(-c3ccc(-n5c6ccccc6c6ccccc65)cc3)cc2O4)cc1]

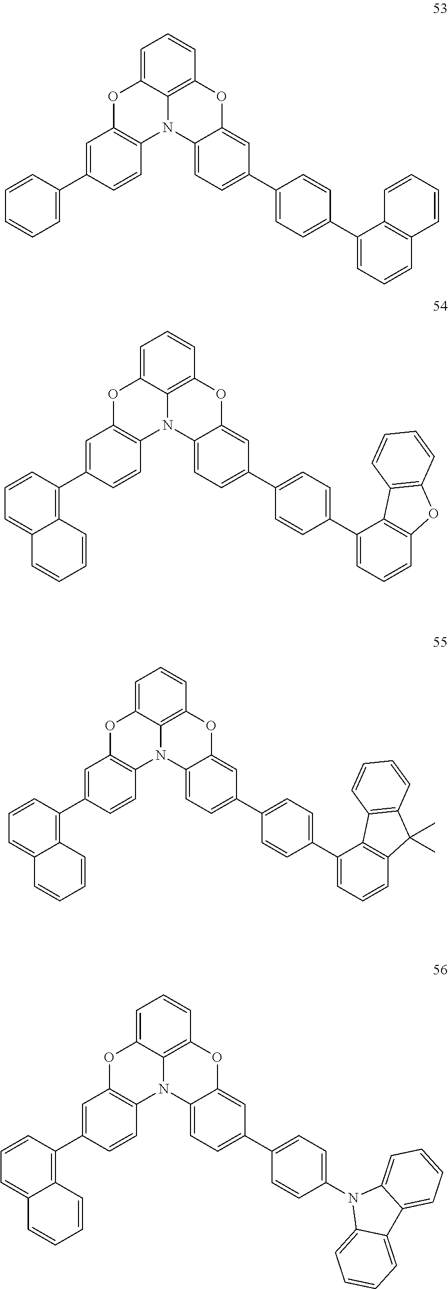 [SMILES: c1ccc(-c2ccc3c(c2)Oc2cccc4c2N3c2ccc(-c3ccc(-c5cccc6ccccc56)cc3)cc2O4)cc1 ; c1cc2c3c(c1)Oc1cc(-c4cccc5ccccc45)ccc1N3c1ccc(-c3ccc(-c4cccc5oc6ccccc6c45)cc3)cc1O2 ; CC1(C)c2ccccc2-c2c(-c3ccc(-c4ccc5c(c4)Oc4cccc6c4N5c4ccc(-c5cccc7ccccc57)cc4O6)cc3)cccc21 ; c1cc2c3c(c1)Oc1cc(-c4cccc5ccccc45)ccc1N3c1ccc(-c3ccc(-n4c5ccccc5c5ccccc54)cc3)cc1O2]

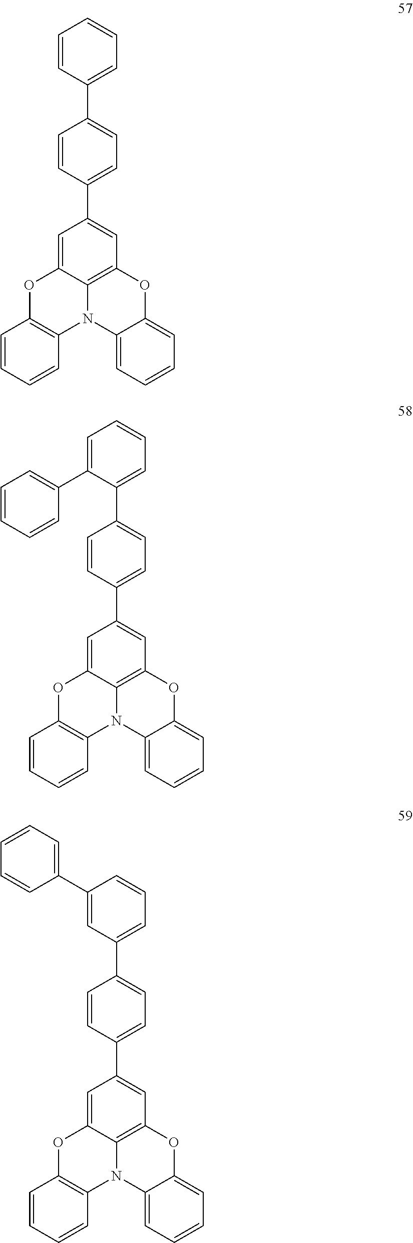

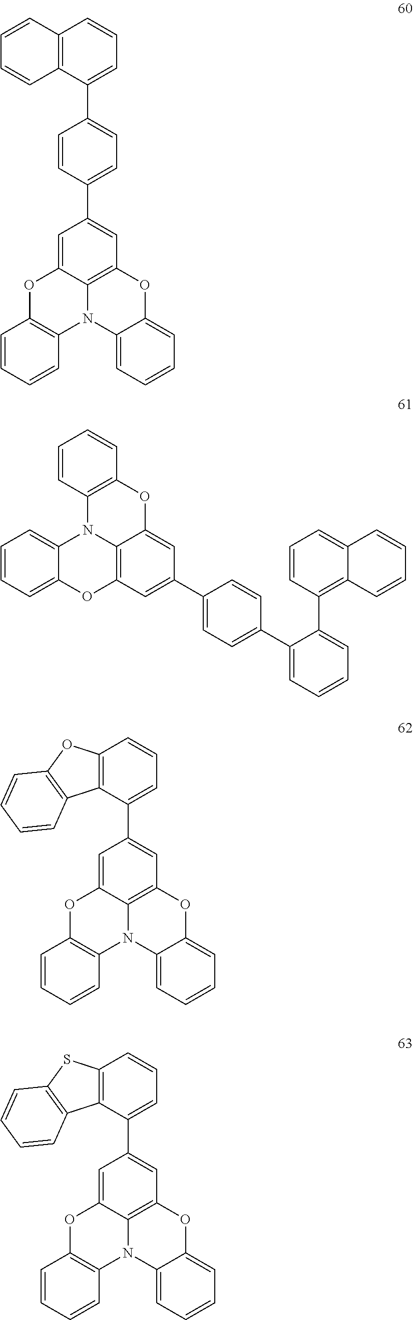 [SMILES: c1ccc2c(c1)Oc1cc(-c3ccc(-c4cccc5ccccc45)cc3)cc3c1N2c1ccccc1O3 ; c1ccc2c(c1)Oc1cc(-c3ccc(-c4ccccc4-c4cccc5ccccc45)cc3)cc3c1N2c1ccccc1O3 ; c1ccc2c(c1)Oc1cc(-c3cccc4oc5ccccc5c34)cc3c1N2c1ccccc1O3 ; c1ccc2c(c1)Oc1cc(-c3cccc4sc5ccccc5c34)cc3c1N2c1ccccc1O3]

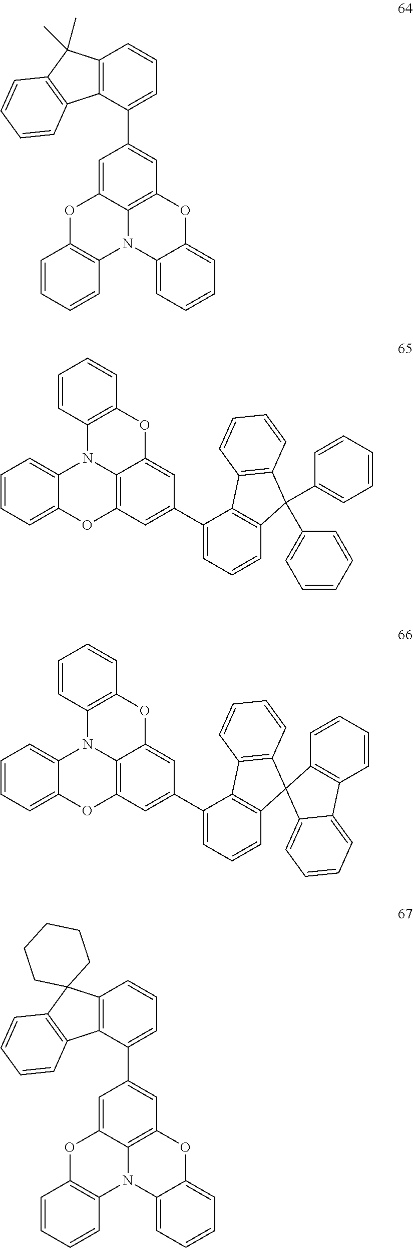 [SMILES: CC1(C)c2ccccc2-c2c(-c3cc4c5c(c3)Oc3ccccc3N5c3ccccc3O4)cccc21 ; c1ccc(C2(c3ccccc3)c3ccccc3-c3c(-c4cc5c6c(c4)Oc4ccccc4N6c4ccccc4O5)cccc32)cc1 ; c1ccc2c(c1)Oc1cc(-c3cccc4c3-c3ccccc3C43c4ccccc4-c4ccccc43)cc3c1N2c1ccccc1O3 ; c1ccc2c(c1)Oc1cc(-c3cccc4c3-c3ccccc3C43CCCCC3)cc3c1N2c1ccccc1O3]

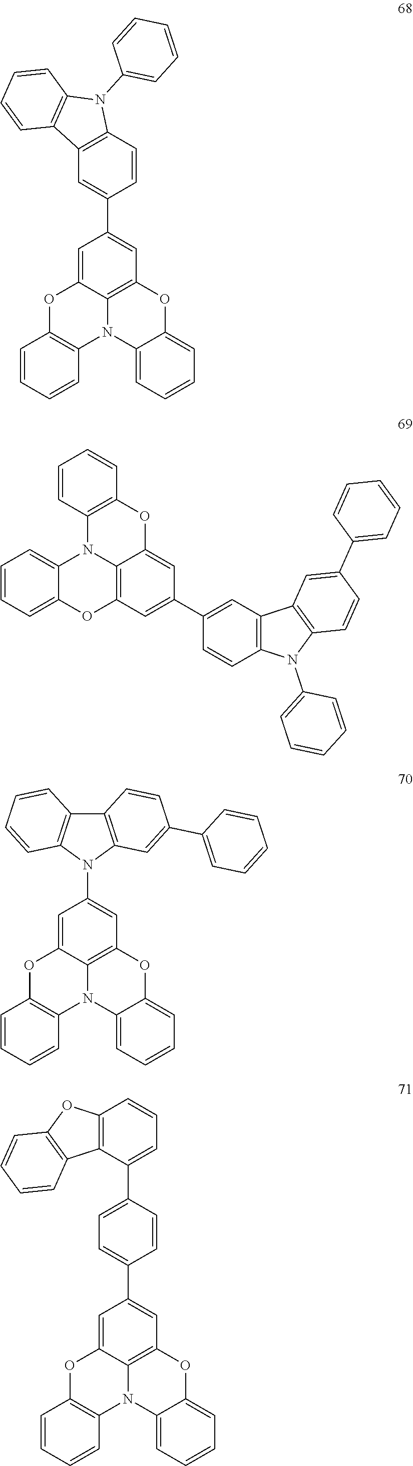

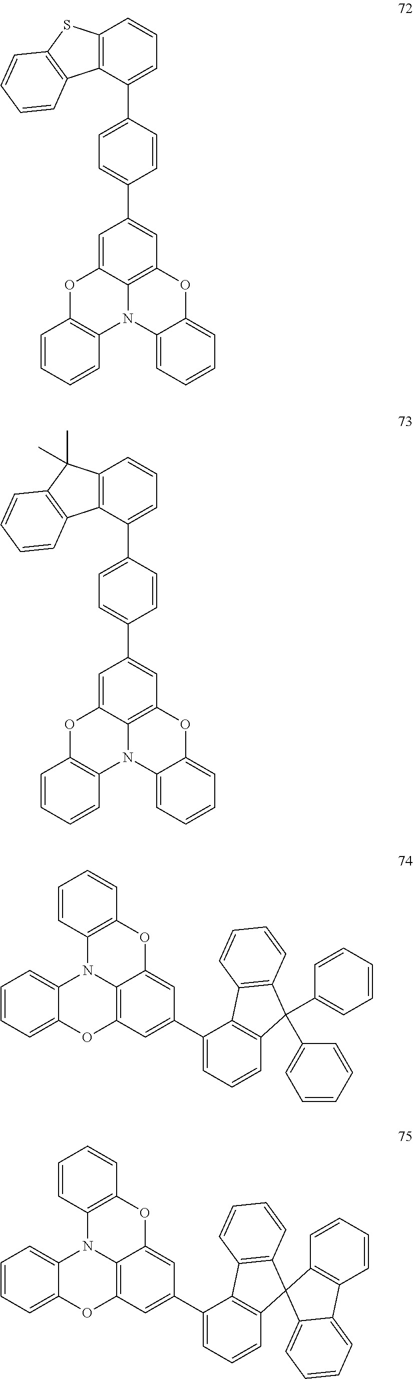

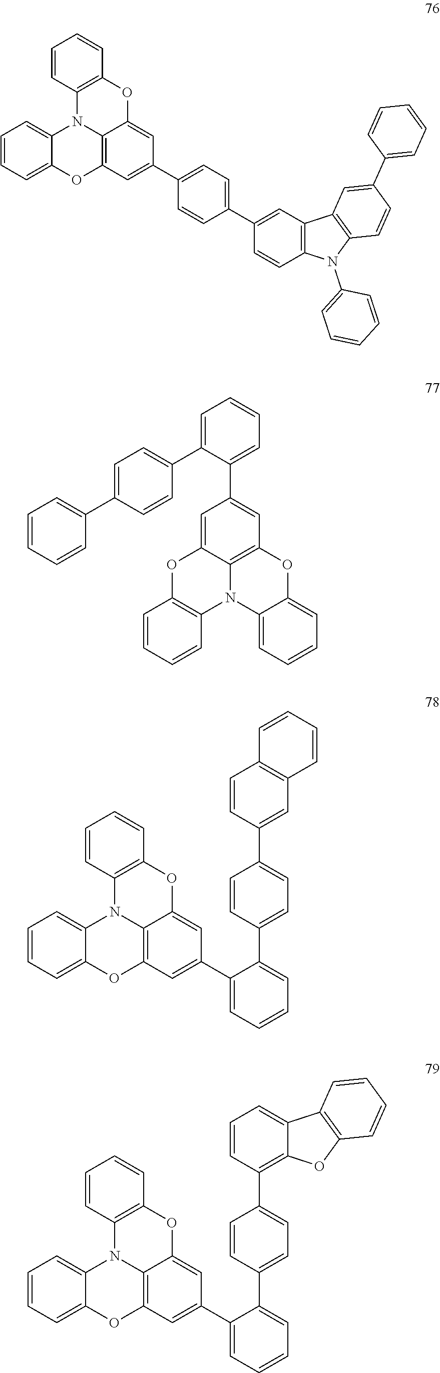 [SMILES: c1ccc(-c2ccc3c(c2)c2cc(-c4ccc(-c5cc6c7c(c5)Oc5ccccc5N7c5ccccc5O6)cc4)ccc2n3-c2ccccc2)cc1 ; c1ccc(-c2ccc(-c3ccccc3-c3cc4c5c(c3)Oc3ccccc3N5c3ccccc3O4)cc2)cc1 ; c1ccc2c(c1)Oc1cc(-c3ccccc3-c3ccc(-c4ccc5ccccc5c4)cc3)cc3c1N2c1ccccc1O3 ; c1ccc2c(c1)Oc1cc(-c3ccccc3-c3ccc(-c4cccc5c4oc4ccccc45)cc3)cc3c1N2c1ccccc1O3]

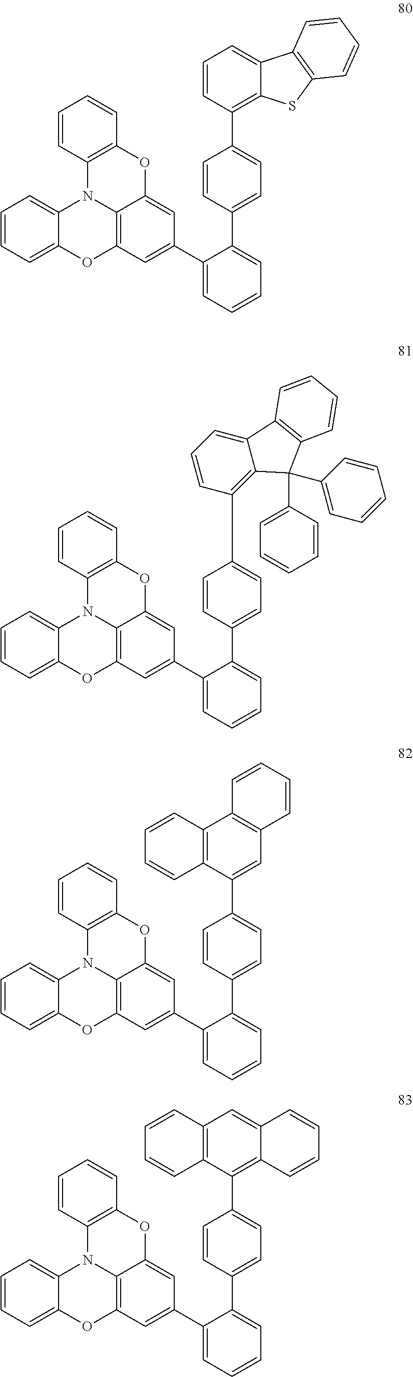

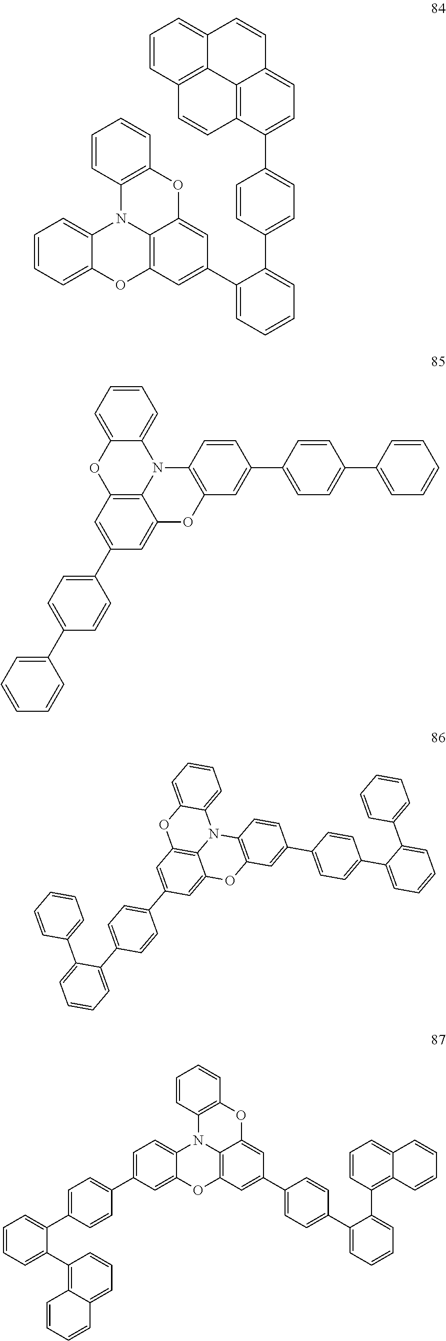 [SMILES: c1ccc2c(c1)Oc1cc(-c3ccccc3-c3ccc(-c4ccc5ccc6cccc7ccc4c5c67)cc3)cc3c1N2c1ccccc1O3 ; c1ccc(-c2ccc(-c3ccc4c(c3)Oc3cc(-c5ccc(-c6ccccc6)cc5)cc5c3N4c3ccccc3O5)cc2)cc1 ; c1ccc(-c2ccccc2-c2ccc(-c3ccc4c(c3)Oc3cc(-c5ccc(-c6ccccc6-c6ccccc6)cc5)cc5c3N4c3ccccc3O5)cc2)cc1 ; c1ccc2c(c1)Oc1cc(-c3ccc(-c4ccccc4-c4cccc5ccccc45)cc3)cc3c1N2c1ccc(-c2ccc(-c4ccccc4-c4cccc5ccccc45)cc2)cc1O3]

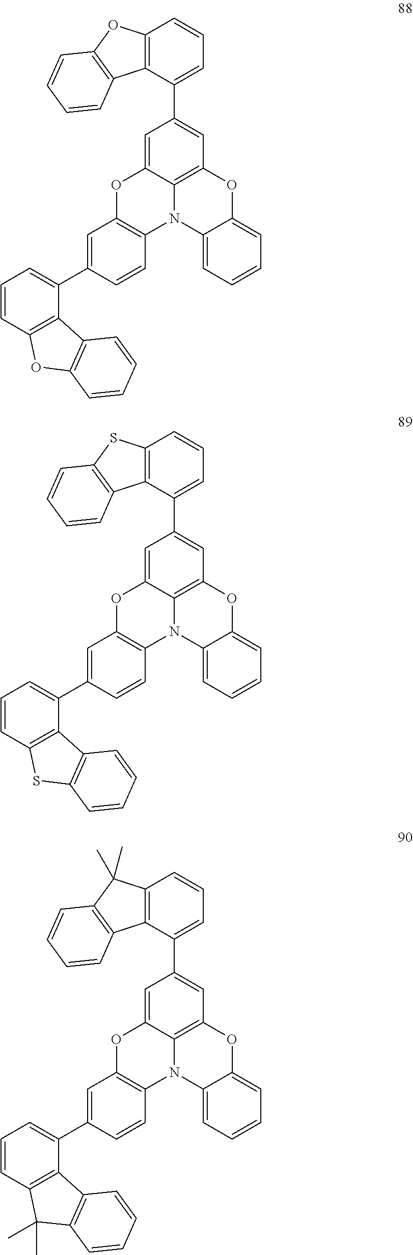

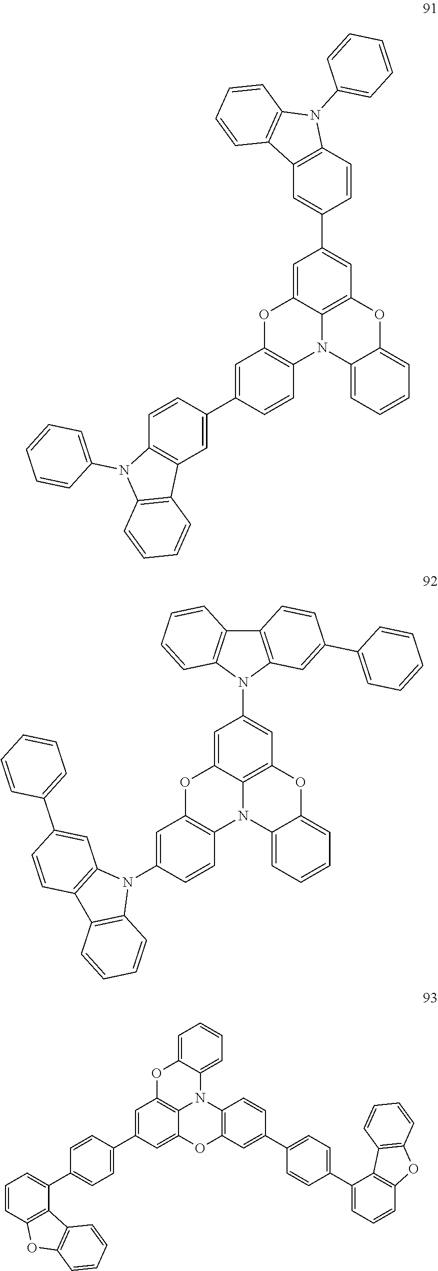 [SMILES: c1ccc(-n2c3ccccc3c3cc(-c4ccc5c(c4)Oc4cc(-c6ccc7c(c6)c6ccccc6n7-c6ccccc6)cc6c4N5c4ccccc4O6)ccc32)cc1 ; c1ccc(-c2ccc3c4ccccc4n(-c4ccc5c(c4)Oc4cc(-n6c7ccccc7c7ccc(-c8ccccc8)cc76)cc6c4N5c4ccccc4O6)c3c2)cc1 ; c1ccc2c(c1)Oc1cc(-c3ccc(-c4cccc5oc6ccccc6c45)cc3)cc3c1N2c1ccc(-c2ccc(-c4cccc5oc6ccccc6c45)cc2)cc1O3]

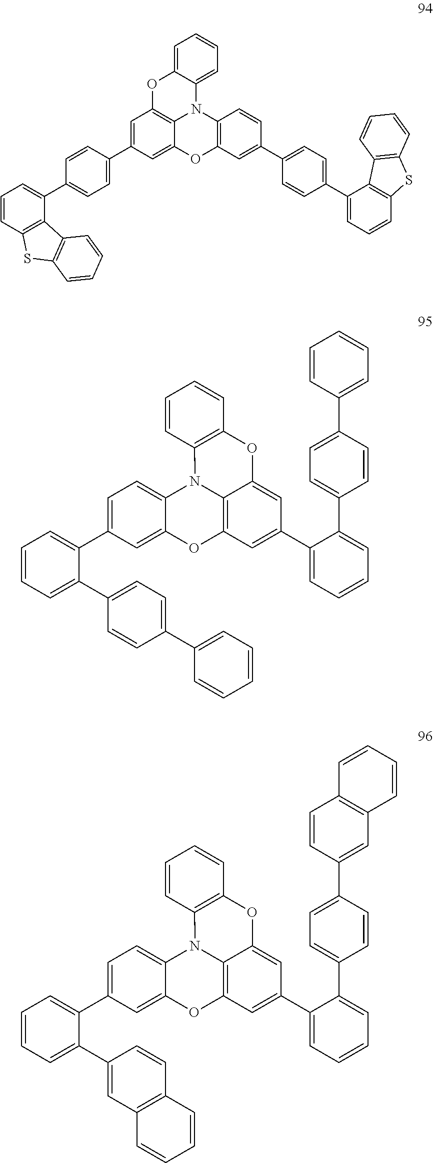 [SMILES: c1ccc2c(c1)Oc1cc(-c3ccc(-c4cccc5sc6ccccc6c45)cc3)cc3c1N2c1ccc(-c2ccc(-c4cccc5sc6ccccc6c45)cc2)cc1O3 ; c1ccc(-c2ccc(-c3ccccc3-c3ccc4c(c3)Oc3cc(-c5ccccc5-c5ccc(-c6ccccc6)cc5)cc5c3N4c3ccccc3O5)cc2)cc1 ; c1ccc2c(c1)Oc1cc(-c3ccccc3-c3ccc(-c4ccc5ccccc5c4)cc3)cc3c1N2c1ccc(-c2ccccc2-c2ccc4ccccc4c2)cc1O3]

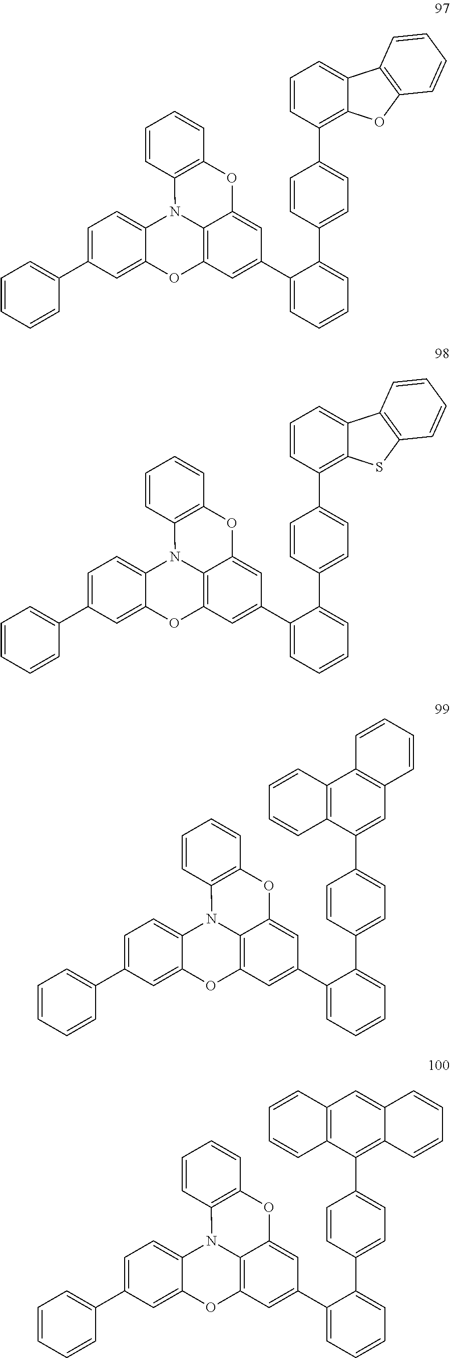 [SMILES: c1ccc(-c2ccc3c(c2)Oc2cc(-c4ccccc4-c4ccc(-c5cccc6c5oc5ccccc56)cc4)cc4c2N3c2ccccc2O4)cc1 ; c1ccc(-c2ccc3c(c2)Oc2cc(-c4ccccc4-c4ccc(-c5cccc6c5sc5ccccc56)cc4)cc4c2N3c2ccccc2O4)cc1 ; c1ccc(-c2ccc3c(c2)Oc2cc(-c4ccccc4-c4ccc(-c5cc6ccccc6c6ccccc56)cc4)cc4c2N3c2ccccc2O4)cc1 ; c1ccc(-c2ccc3c(c2)Oc2cc(-c4ccccc4-c4ccc(-c5c6ccccc6cc6ccccc56)cc4)cc4c2N3c2ccccc2O4)cc1]

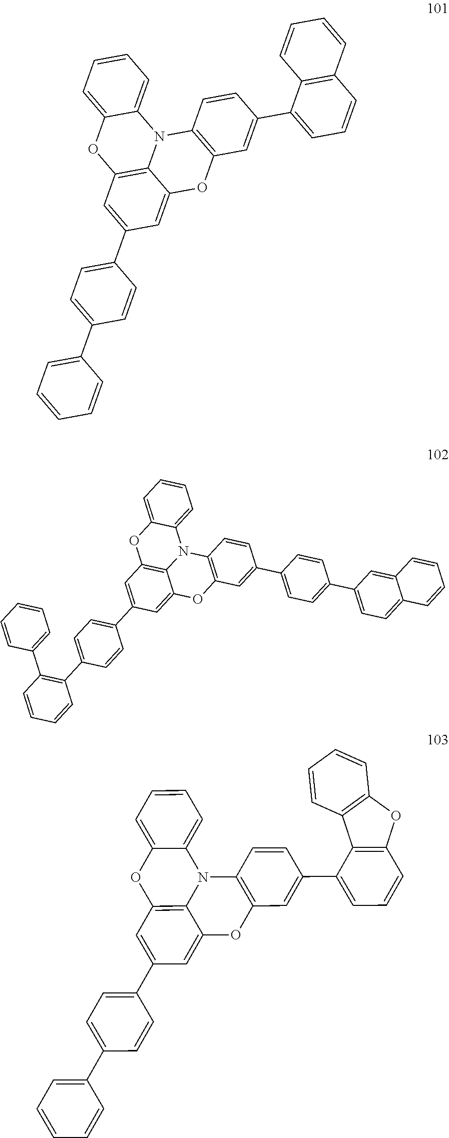 [SMILES: c1ccc(-c2ccc(-c3cc4c5c(c3)Oc3cc(-c6cccc7ccccc67)ccc3N5c3ccccc3O4)cc2)cc1 ; c1ccc(-c2ccccc2-c2ccc(-c3cc4c5c(c3)Oc3cc(-c6ccc(-c7ccc8ccccc8c7)cc6)ccc3N5c3ccccc3O4)cc2)cc1 ; c1ccc(-c2ccc(-c3cc4c5c(c3)Oc3cc(-c6cccc7oc8ccccc8c67)ccc3N5c3ccccc3O4)cc2)cc1]

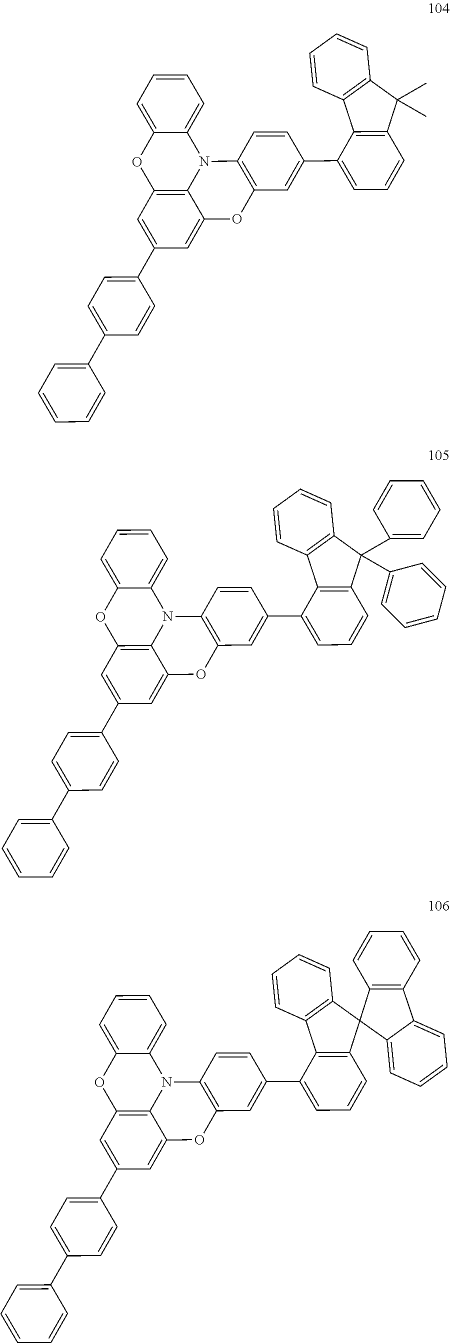 [SMILES: CC1(C)c2ccccc2-c2c(-c3ccc4c(c3)Oc3cc(-c5ccc(-c6ccccc6)cc5)cc5c3N4c3ccccc3O5)cccc21 ; c1ccc(-c2ccc(-c3cc4c5c(c3)Oc3cc(-c6cccc7c6-c6ccccc6C7(c6ccccc6)c6ccccc6)ccc3N5c3ccccc3O4)cc2)cc1 ; c1ccc(-c2ccc(-c3cc4c5c(c3)Oc3cc(-c6cccc7c6-c6ccccc6C76c7ccccc7-c7ccccc76)ccc3N5c3ccccc3O4)cc2)cc1]

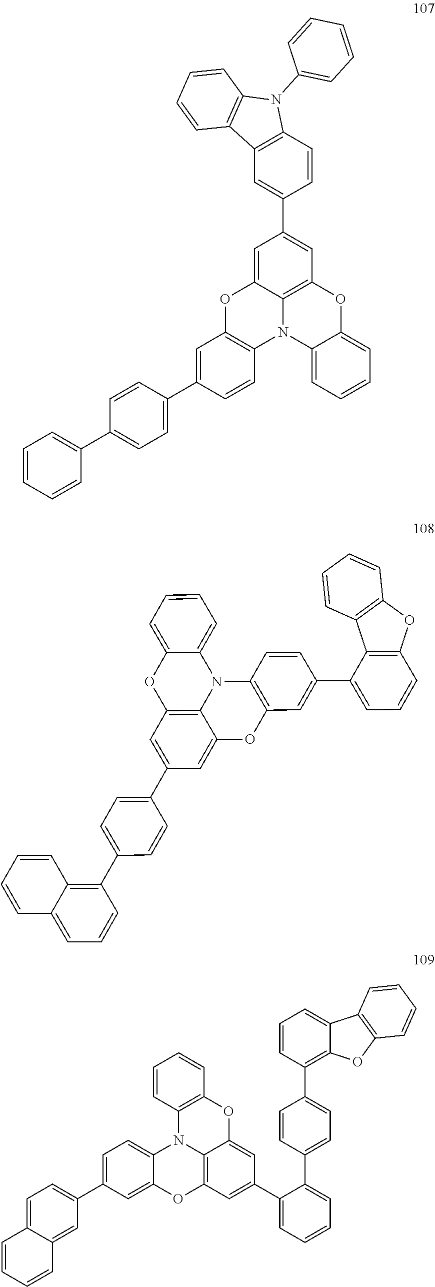 [SMILES: c1ccc(-c2ccc(-c3ccc4c(c3)Oc3cc(-c5ccc6c(c5)c5ccccc5n6-c5ccccc5)cc5c3N4c3ccccc3O5)cc2)cc1 ; c1ccc2c(c1)Oc1cc(-c3ccc(-c4cccc5ccccc45)cc3)cc3c1N2c1ccc(-c2cccc4oc5ccccc5c24)cc1O3 ; c1ccc2c(c1)Oc1cc(-c3ccccc3-c3ccc(-c4cccc5c4oc4ccccc45)cc3)cc3c1N2c1ccc(-c2ccc4ccccc4c2)cc1O3]

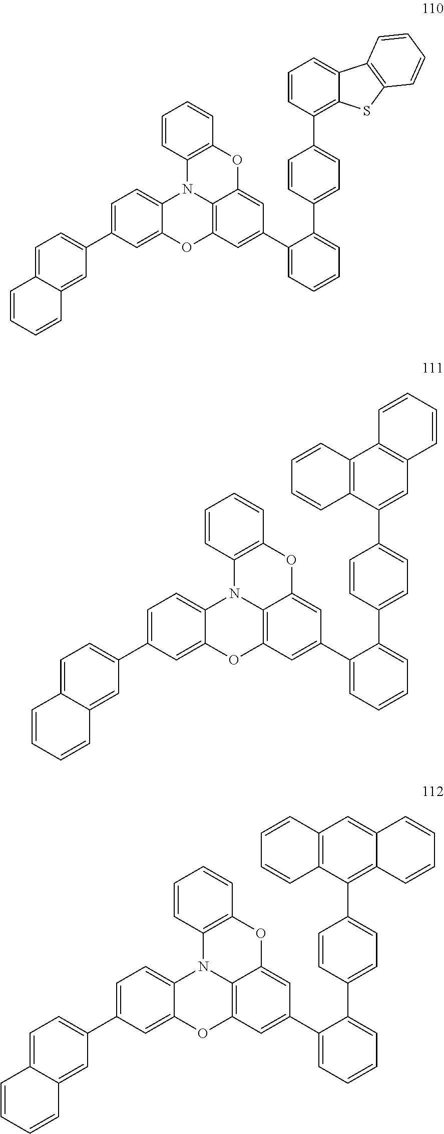 [SMILES: c1ccc2c(c1)Oc1cc(-c3ccccc3-c3ccc(-c4cccc5c4sc4ccccc45)cc3)cc3c1N2c1ccc(-c2ccc4ccccc4c2)cc1O3 ; c1ccc2c(c1)Oc1cc(-c3ccccc3-c3ccc(-c4cc5ccccc5c5ccccc45)cc3)cc3c1N2c1ccc(-c2ccc4ccccc4c2)cc1O3 ; c1ccc2c(c1)Oc1cc(-c3ccccc3-c3ccc(-c4c5ccccc5cc5ccccc45)cc3)cc3c1N2c1ccc(-c2ccc4ccccc4c2)cc1O3]

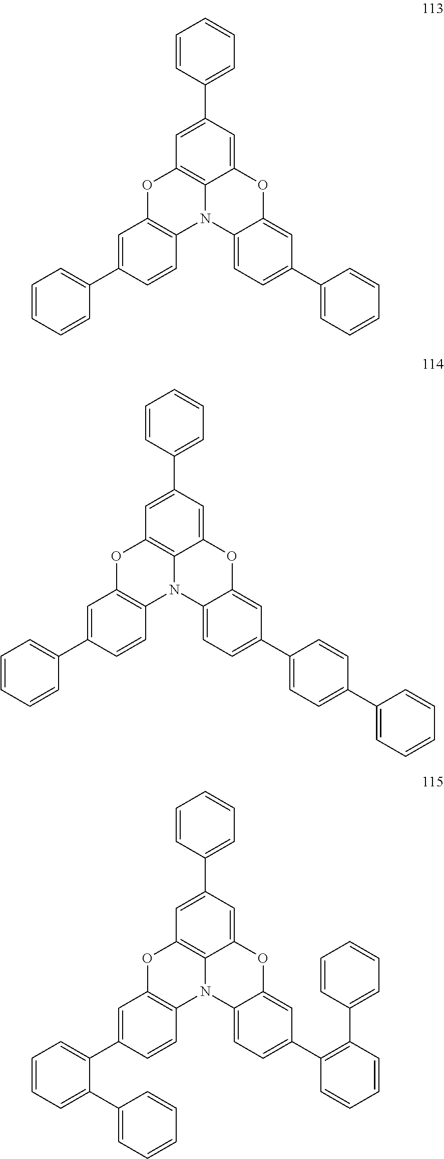 [SMILES: c1ccc(-c2ccc3c(c2)Oc2cc(-c4ccccc4)cc4c2N3c2ccc(-c3ccccc3)cc2O4)cc1 ; c1ccc(-c2ccc(-c3ccc4c(c3)Oc3cc(-c5ccccc5)cc5c3N4c3ccc(-c4ccccc4)cc3O5)cc2)cc1 ; c1ccc(-c2cc3c4c(c2)Oc2cc(-c5ccccc5-c5ccccc5)ccc2N4c2ccc(-c4ccccc4-c4ccccc4)cc2O3)cc1]

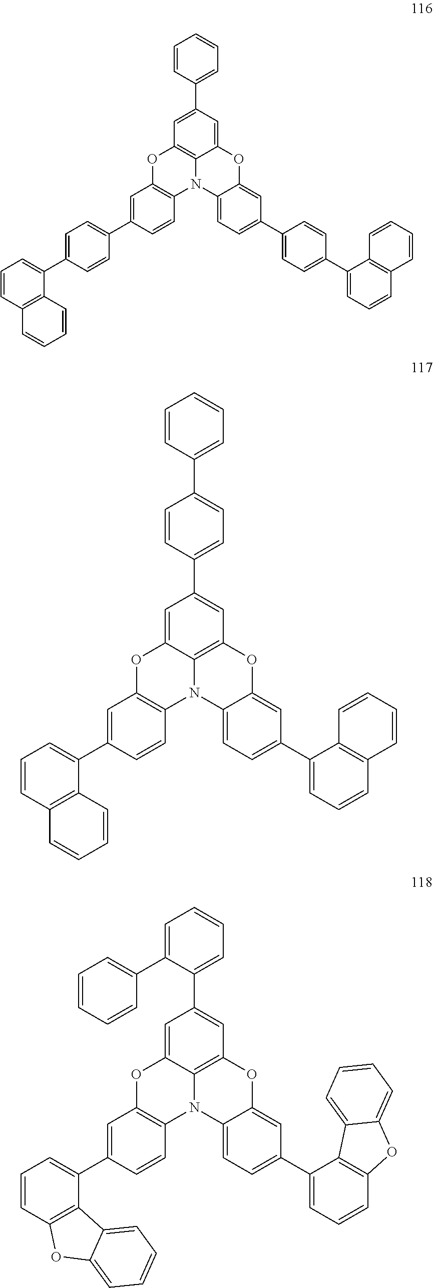 [SMILES: c1ccc(-c2cc3c4c(c2)Oc2cc(-c5ccc(-c6cccc7ccccc67)cc5)ccc2N4c2ccc(-c4ccc(-c5cccc6ccccc56)cc4)cc2O3)cc1 ; c1ccc(-c2ccc(-c3cc4c5c(c3)Oc3cc(-c6cccc7ccccc67)ccc3N5c3ccc(-c5cccc6ccccc56)cc3O4)cc2)cc1 ; c1ccc(-c2ccccc2-c2cc3c4c(c2)Oc2cc(-c5cccc6oc7ccccc7c56)ccc2N4c2ccc(-c4cccc5oc6ccccc6c45)cc2O3)cc1]

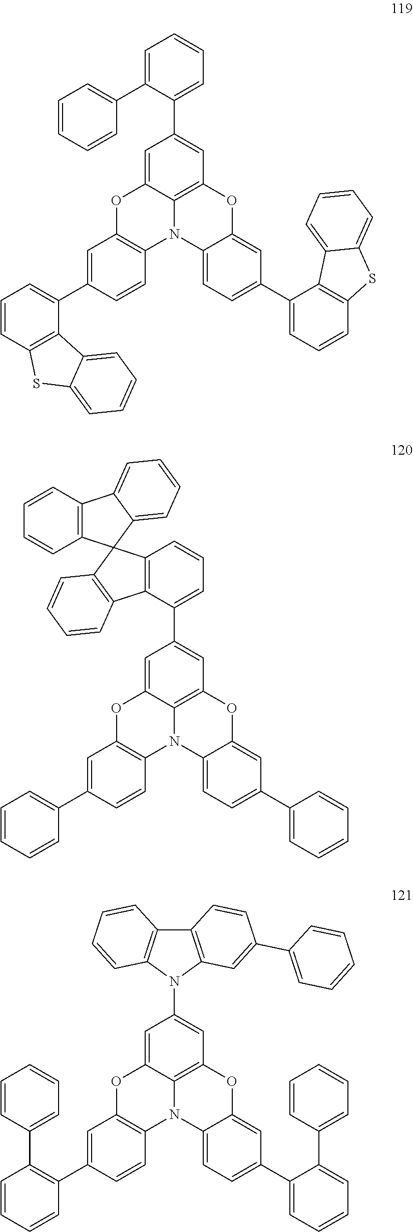 [SMILES: c1ccc(-c2ccccc2-c2cc3c4c(c2)Oc2cc(-c5cccc6sc7ccccc7c56)ccc2N4c2ccc(-c4cccc5sc6ccccc6c45)cc2O3)cc1 ; c1ccc(-c2ccc3c(c2)Oc2cc(-c4cccc5c4-c4ccccc4C54c5ccccc5-c5ccccc54)cc4c2N3c2ccc(-c3ccccc3)cc2O4)cc1 ; c1ccc(-c2ccc3c4ccccc4n(-c4cc5c6c(c4)Oc4cc(-c7ccccc7-c7ccccc7)ccc4N6c4ccc(-c6ccccc6-c6ccccc6)cc4O5)c3c2)cc1]

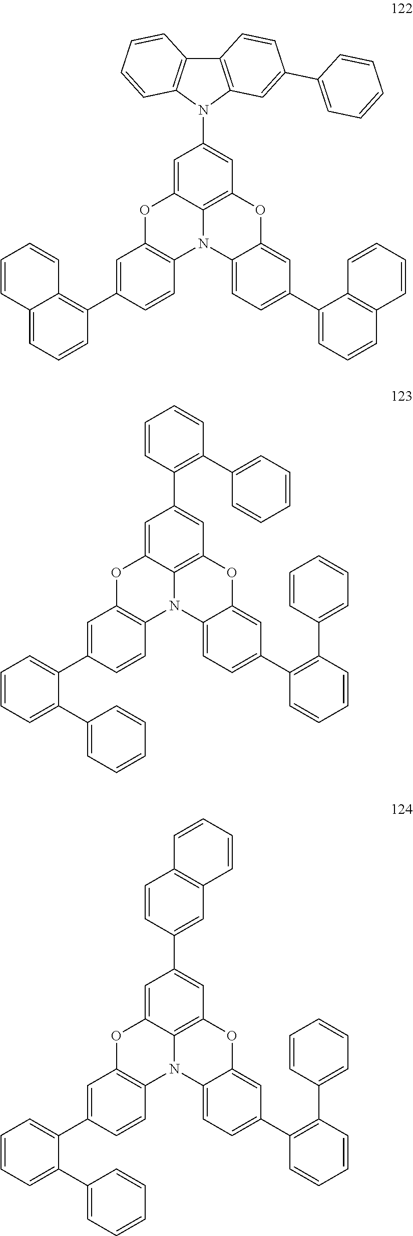 [SMILES: c1ccc(-c2ccc3c4ccccc4n(-c4cc5c6c(c4)Oc4cc(-c7cccc8ccccc78)ccc4N6c4ccc(-c6cccc7ccccc67)cc4O5)c3c2)cc1 ; c1ccc(-c2ccccc2-c2ccc3c(c2)Oc2cc(-c4ccccc4-c4ccccc4)cc4c2N3c2ccc(-c3ccccc3-c3ccccc3)cc2O4)cc1 ; c1ccc(-c2ccccc2-c2ccc3c(c2)Oc2cc(-c4ccc5ccccc5c4)cc4c2N3c2ccc(-c3ccccc3-c3ccccc3)cc2O4)cc1]

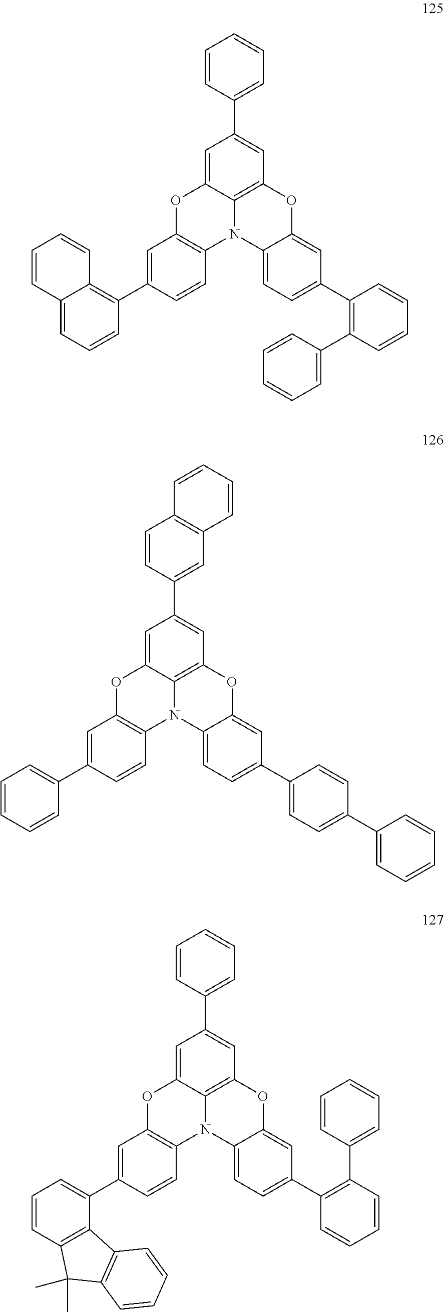 [SMILES: c1ccc(-c2cc3c4c(c2)Oc2cc(-c5cccc6ccccc56)ccc2N4c2ccc(-c4ccccc4-c4ccccc4)cc2O3)cc1 ; c1ccc(-c2ccc(-c3ccc4c(c3)Oc3cc(-c5ccc6ccccc6c5)cc5c3N4c3ccc(-c4ccccc4)cc3O5)cc2)cc1 ; CC1(C)c2ccccc2-c2c(-c3ccc4c(c3)Oc3cc(-c5ccccc5)cc5c3N4c3ccc(-c4ccccc4-c4ccccc4)cc3O5)cccc21]

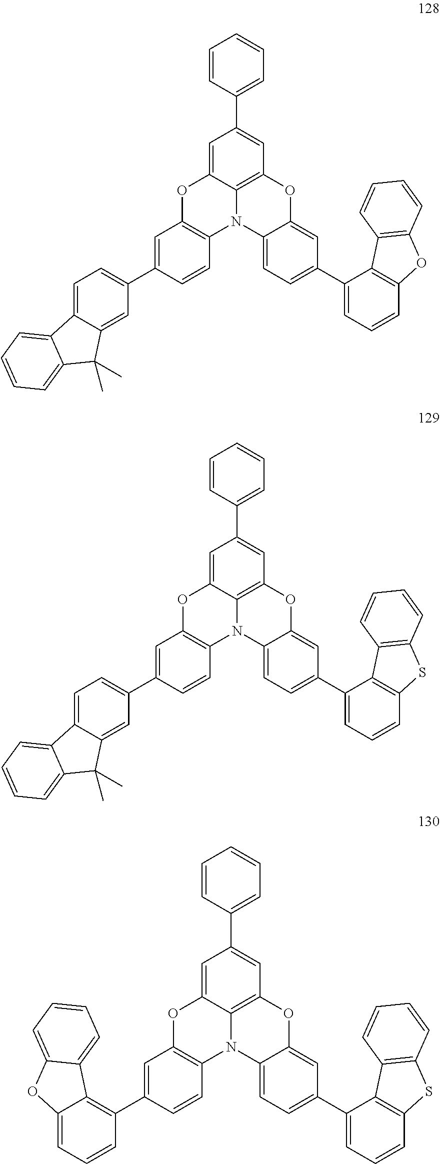 [SMILES: CC1(C)c2ccccc2-c2ccc(-c3ccc4c(c3)Oc3cc(-c5ccccc5)cc5c3N4c3ccc(-c4cccc6oc7ccccc7c46)cc3O5)cc21 ; CC1(C)c2ccccc2-c2ccc(-c3ccc4c(c3)Oc3cc(-c5ccccc5)cc5c3N4c3ccc(-c4cccc6sc7ccccc7c46)cc3O5)cc21 ; c1ccc(-c2cc3c4c(c2)Oc2cc(-c5cccc6sc7ccccc7c56)ccc2N4c2ccc(-c4cccc5oc6ccccc6c45)cc2O3)cc1]

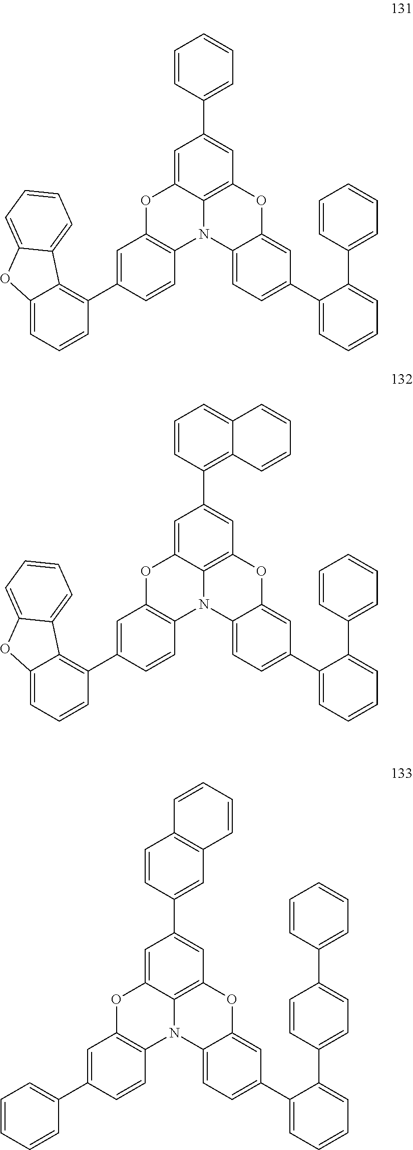 [SMILES: c1ccc(-c2cc3c4c(c2)Oc2cc(-c5cccc6oc7ccccc7c56)ccc2N4c2ccc(-c4ccccc4-c4ccccc4)cc2O3)cc1 ; c1ccc(-c2ccccc2-c2ccc3c(c2)Oc2cc(-c4cccc5ccccc45)cc4c2N3c2ccc(-c3cccc5oc6ccccc6c35)cc2O4)cc1 ; c1ccc(-c2ccc(-c3ccccc3-c3ccc4c(c3)Oc3cc(-c5ccc6ccccc6c5)cc5c3N4c3ccc(-c4ccccc4)cc3O5)cc2)cc1]

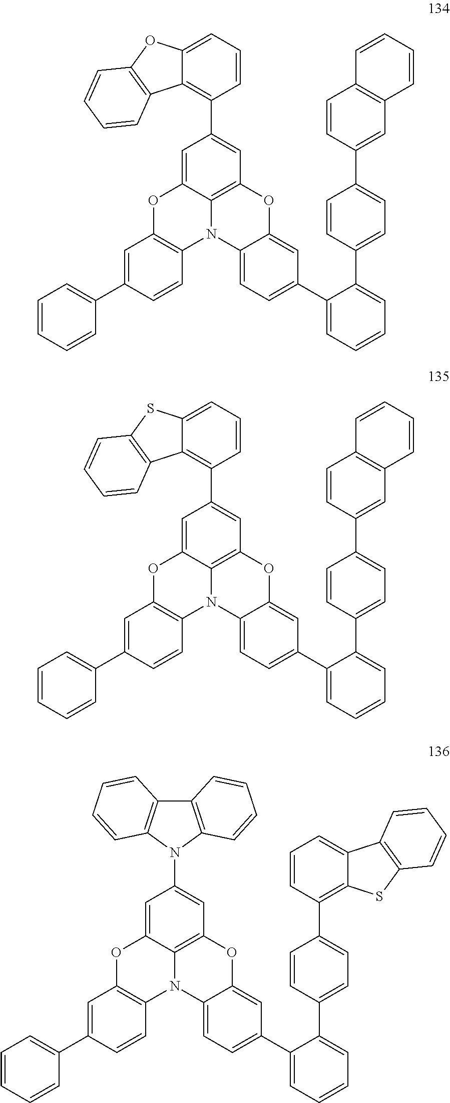 [SMILES: c1ccc(-c2ccc3c(c2)Oc2cc(-c4cccc5oc6ccccc6c45)cc4c2N3c2ccc(-c3ccccc3-c3ccc(-c5ccc6ccccc6c5)cc3)cc2O4)cc1 ; c1ccc(-c2ccc3c(c2)Oc2cc(-c4cccc5sc6ccccc6c45)cc4c2N3c2ccc(-c3ccccc3-c3ccc(-c5ccc6ccccc6c5)cc3)cc2O4)cc1 ; c1ccc(-c2ccc3c(c2)Oc2cc(-n4c5ccccc5c5ccccc54)cc4c2N3c2ccc(-c3ccccc3-c3ccc(-c5cccc6c5sc5ccccc56)cc3)cc2O4)cc1]

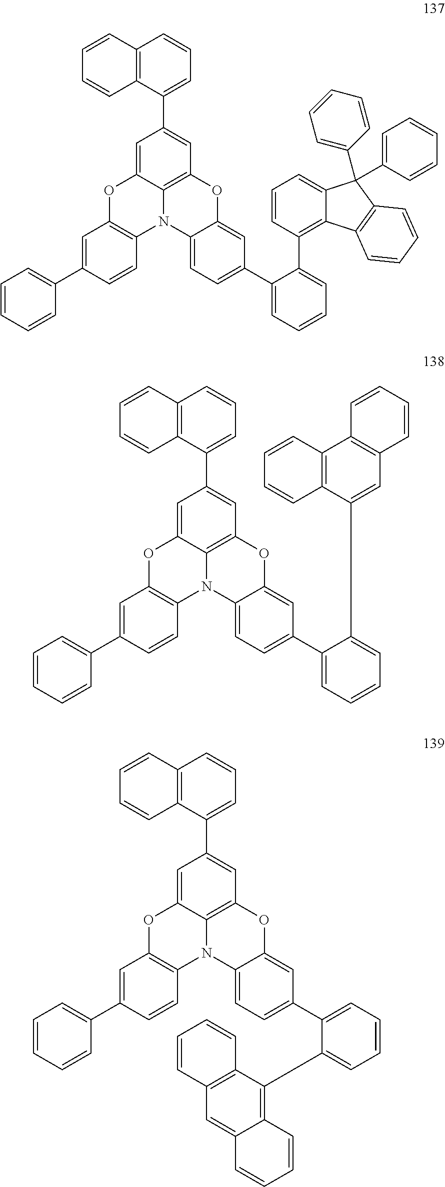 [SMILES: c1ccc(-c2ccc3c(c2)Oc2cc(-c4cccc5ccccc45)cc4c2N3c2ccc(-c3ccccc3-c3cccc5c3-c3ccccc3C5(c3ccccc3)c3ccccc3)cc2O4)cc1 ; c1ccc(-c2ccc3c(c2)Oc2cc(-c4cccc5ccccc45)cc4c2N3c2ccc(-c3ccccc3CCCc3cc5ccccc5c5ccccc35)cc2O4)cc1 ; c1ccc(-c2ccc3c(c2)Oc2cc(-c4cccc5ccccc45)cc4c2N3c2ccc(-c3ccccc3Cc3c5ccccc5cc5ccccc35)cc2O4)cc1]

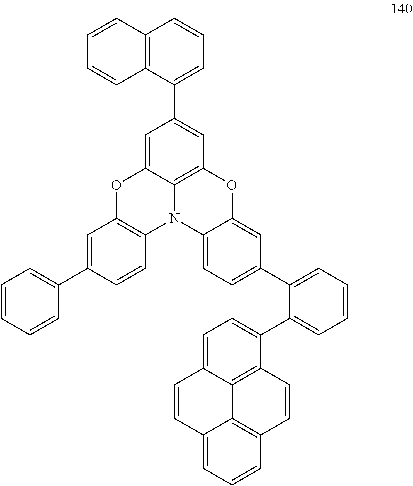 [SMILES: c1ccc(-c2ccc3c(c2)Oc2cc(-c4cccc5ccccc45)cc4c2N3c2ccc(-c3ccccc3-c3ccc5ccc6cccc7ccc3c5c67)cc2O4)cc1]

The hole transport layer of the light-emitting device 10 according to an embodiment may include the condensed cyclic compound represented by Formula 1.

The condensed cyclic compound represented by Formula 1 has a flat structure including a condensed ring and at least one cyclic group as a substituent, resulting in a rigid molecule in terms of bond dissociation energy (BDE). Also, in this regard, the condensed cyclic compound represented by Formula 1 may have a high glass transition temperature (Tg) or a high melting point. Accordingly, the light-emitting device 10 including the hole transport layer that includes the condensed cyclic compound may have high stability.

In the light-emitting device 10 according to an embodiment, the first electrode 110 and the hole transport layer may respectively include the inorganic material and the condensed cyclic compound, thereby having a low progressive driving voltage (ΔV). For example, when the condensed cyclic compound included in the hole transport layer may include a hetero atom, such as nitrogen or oxygen, free electrons of the hetero atom may stabilize the inorganic material which is in an electron deficient state in the first electrode 110, thereby improving the interfacial stability between the first electrode and the hole transport layer. Accordingly, the light-emitting device 10 may have a low progressive driving voltage, high efficiency, and a long lifespan.

In an embodiment, the hole transport layer may be in direct contact with the first electrode 110.

In an embodiment, the hole transport layer may be in direct contact with the third layer of the first electrode 110.

In an embodiment, the hole transport layer may be in direct contact with the emission layer.

In an embodiment, the hole transport layer may consist of the condensed cyclic compound represented by Formula 1.

In an embodiment, the hole transport layer may not include a p-dopant.

In the light-emitting device 10 according to an embodiment, when the hole transport layer does not include a p-dopant, the occurrence of current leakage in the lateral direction due to a p-dopant or the like may be suppressed. Furthermore, a color mixture phenomenon caused by the occurrence of leakage current, may be prevented.

In an embodiment, the absolute value of the HOMO energy level of the hole transport layer may be 5.25 eV or more.

[Emission Layer in Interlayer 130]

When the light-emitting device 10 is a full-color light-emitting device, the emission layer may be patterned into a red emission layer, a green emission layer, and/or a blue emission layer, according to a sub-pixel. In one or more embodiments, the emission layer may have a stacked structure of two or more layers of a red emission layer, a green emission layer, and a blue emission layer, in which the two or more layers contact each other or are separated from each other, to emit white light. In one or more embodiments, the emission layer may include two or more materials of a red light-emitting material, a green light-emitting material, and a blue light-emitting material, in which the two or more materials are mixed with each other in a single layer to emit white light.

The emission layer may include a host and a dopant. The dopant may include a phosphorescent dopant, a fluorescent dopant, or any combination thereof.

An amount of the dopant in the emission layer may be from about 0.01 to about 15 parts by weight based on 100 parts by weight of the host.

In one or more embodiments, the emission layer may include a quantum dot.

The emission layer may include a delayed fluorescence material. The delayed fluorescence material may act as a host or a dopant in the emission layer.

A thickness of the emission layer may be in a range of about 100 Å to about 1,000 Å, for example, about 200 Å to about 600 Å. When the thickness of the emission layer is within these ranges, excellent luminescence characteristics may be obtained without a substantial increase in driving voltage.

[Host]

In an embodiment, the host may include a compound represented by Formula 301:

[Ar₃₀₁]_{xb11}-[(L₃₀₁)_{xb1}-R₃₀₁]_{xb21} Formula 301
wherein, in Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ may respectively be the same as described in connection with Q₁.

For example, when xb11 in Formula 301 is 2 or more, two or more of Ar₃₀₁(s) may be linked to each other via a single bond.

In one or more embodiments, the host may include a compound represented by Formula 301-1, a compound represented by Formula 301-2, or any combination thereof.

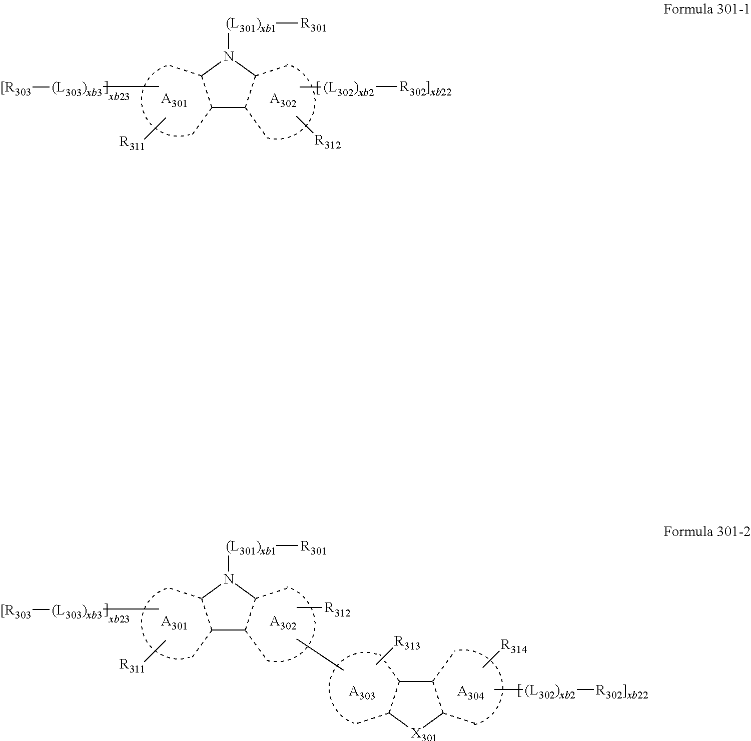

In Formulas 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N-[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may respectively be the same as described herein,
L₃₀₂ to L₃₀₄ may each independently be the same as described in connection with L₃₀₁,
xb2 to xb4 may each independently be the same as described in connection with xb1, and
R₃₀₂ to R₃₀₅ and R₃₁₁ to R₃₁₄ may respectively be the same as described in connection with R₃₀₁.

In one or more embodiments, the host may include an alkali earth metal complex, a post-transition metal complex, or any combination thereof. For example, the host may include a Be complex (for example, Compound H55), an Mg complex, a Zn complex, or any combination thereof.

In an embodiment, the host may include one of Compounds H1 to H124, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), or any combination thereof:

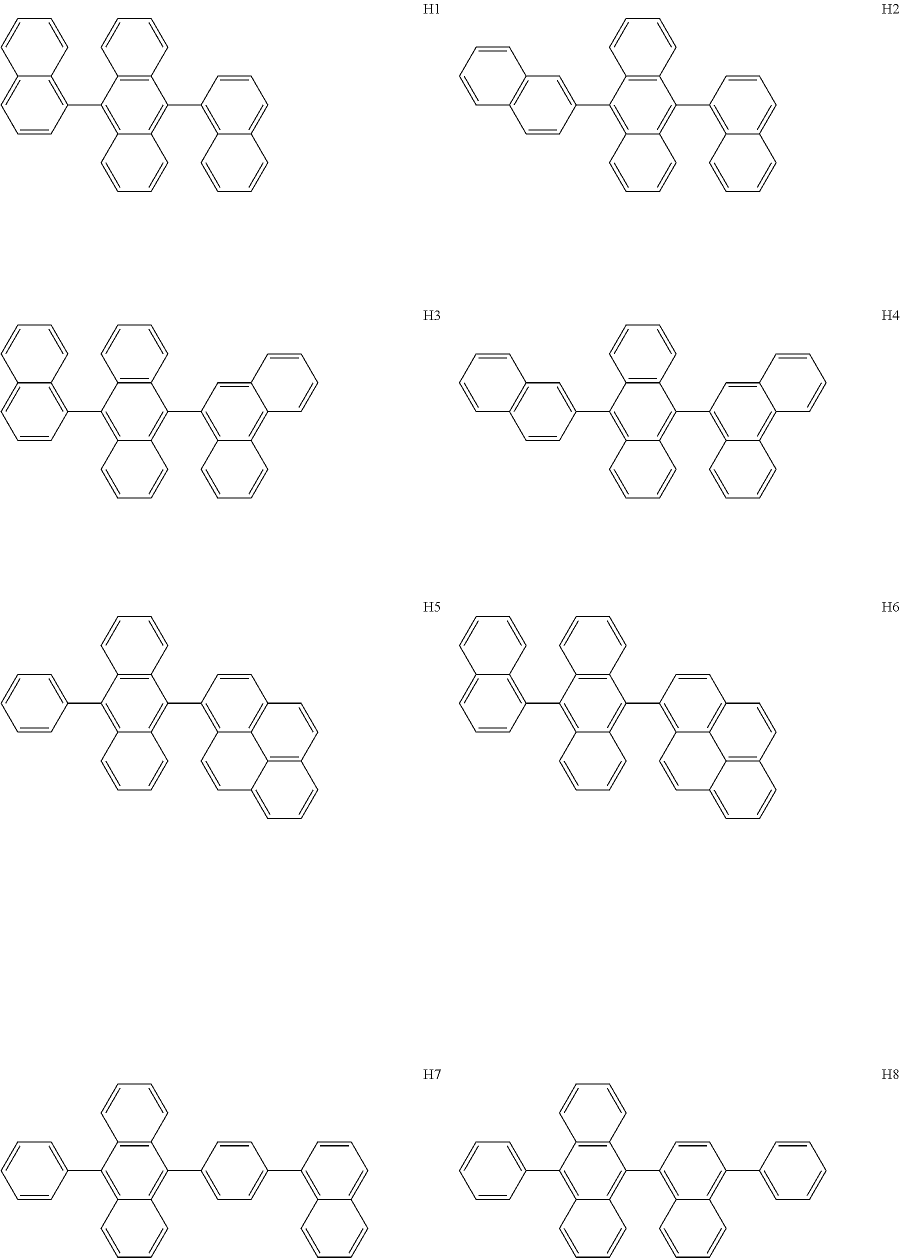

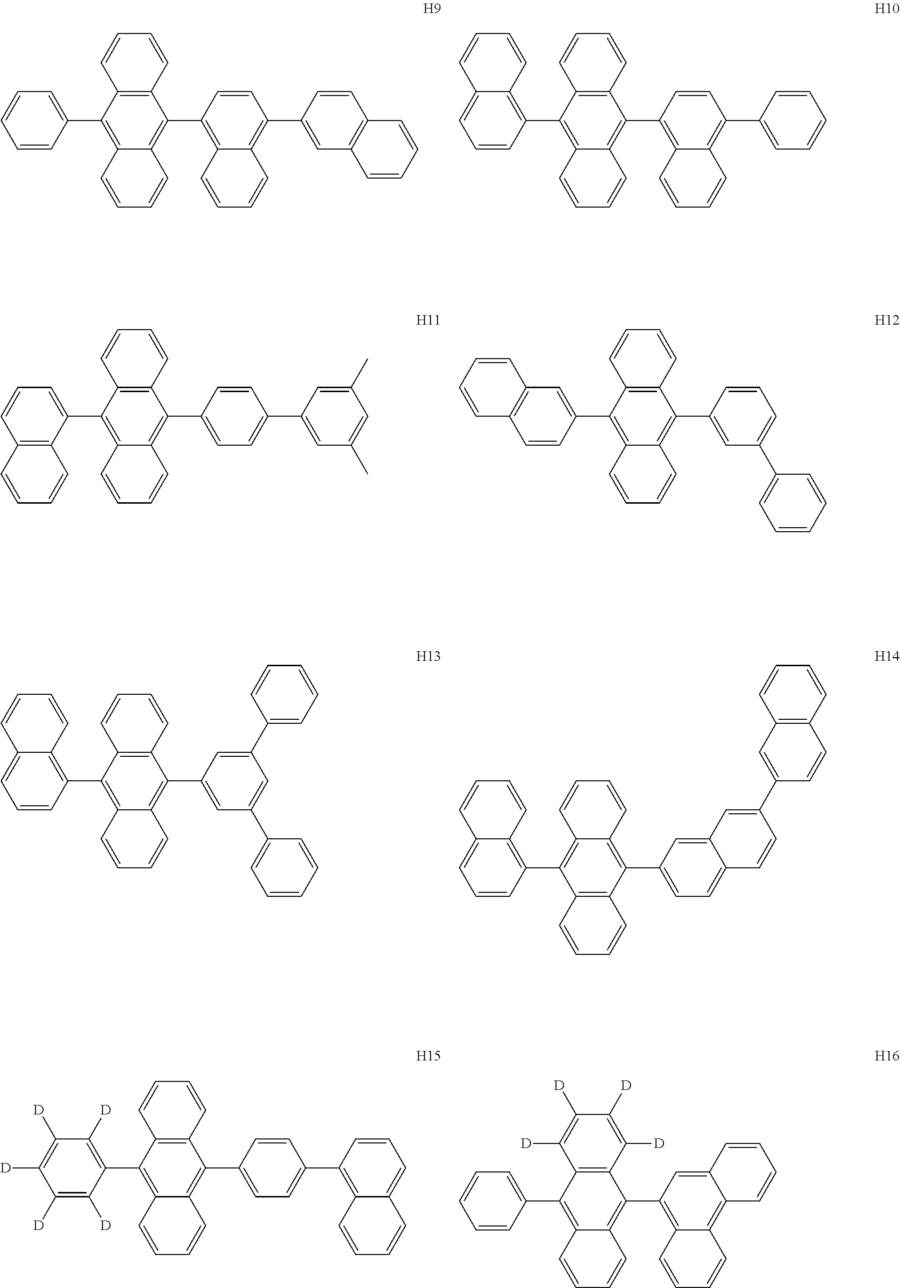

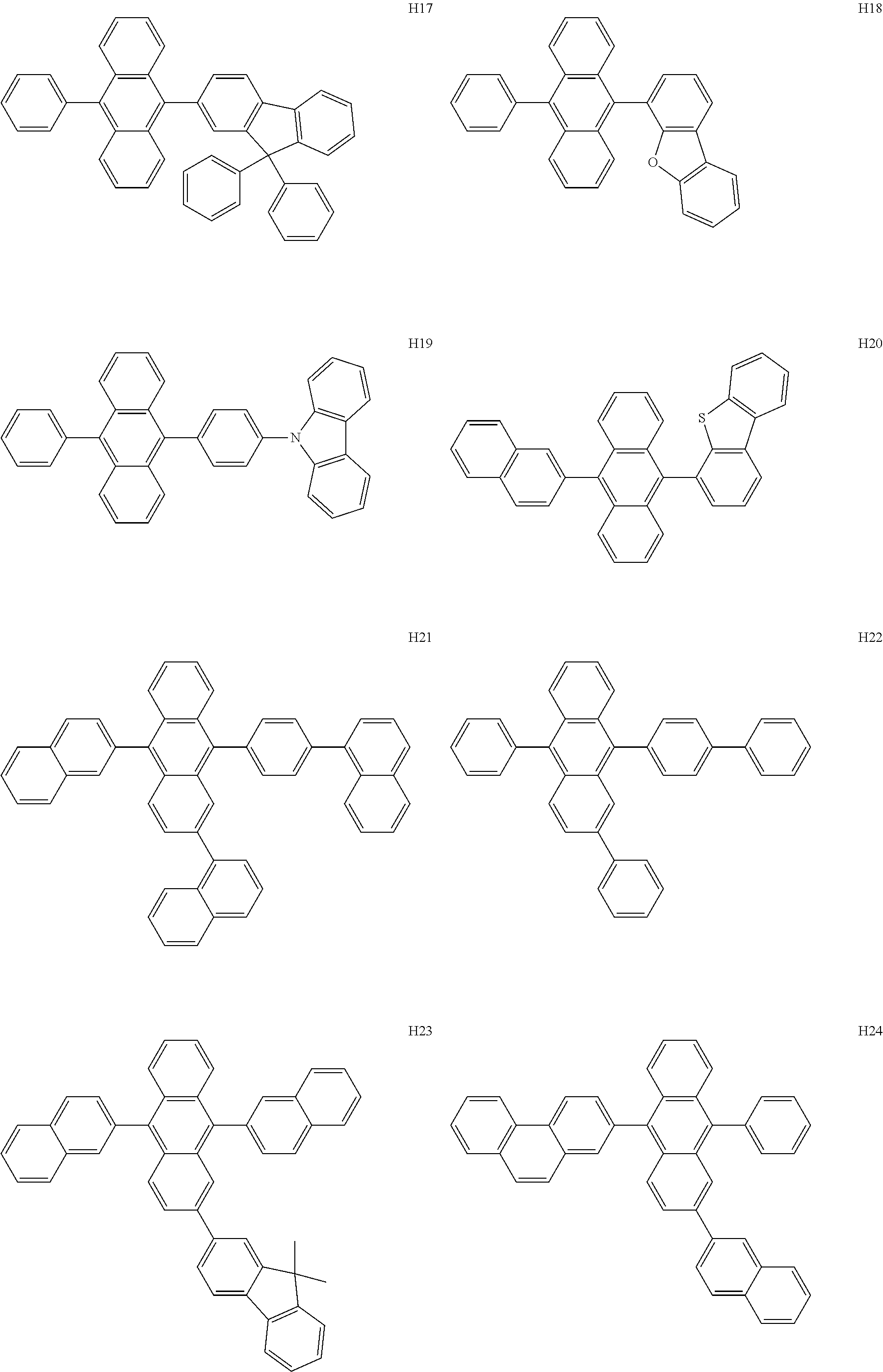

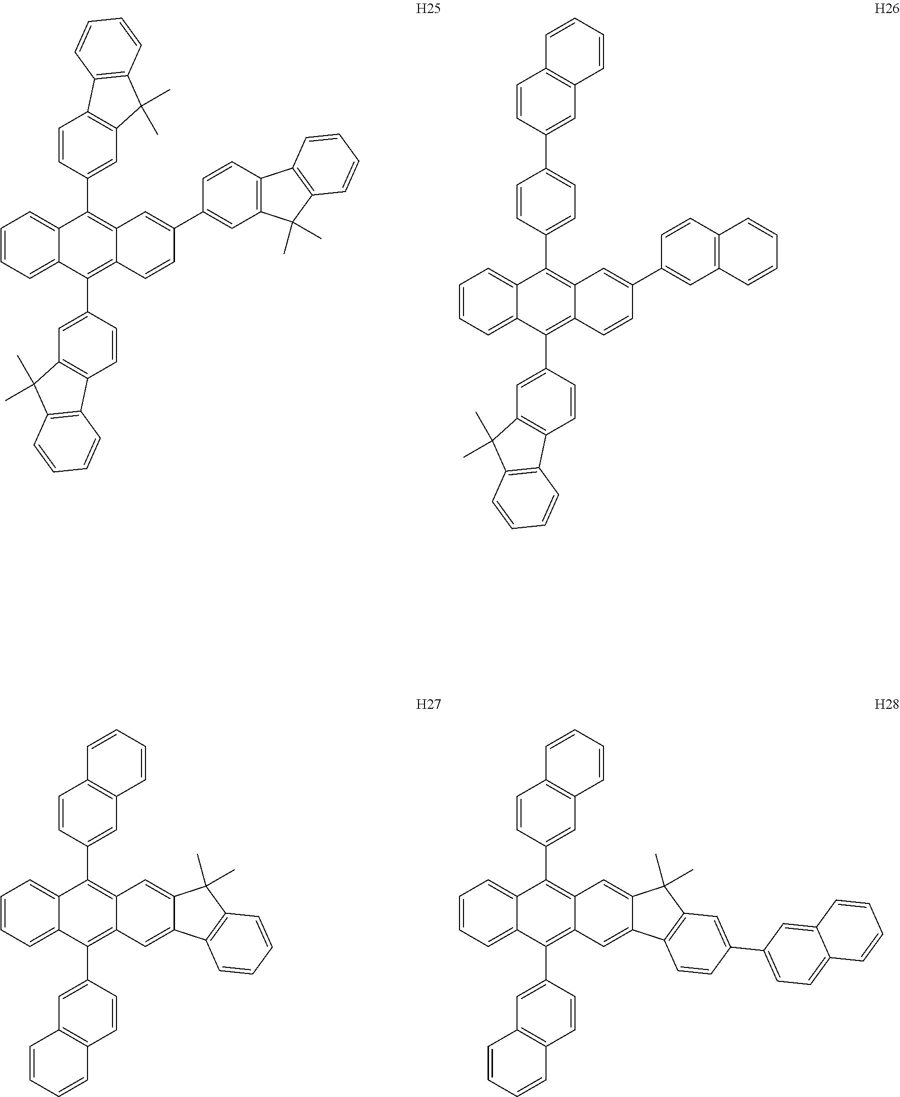

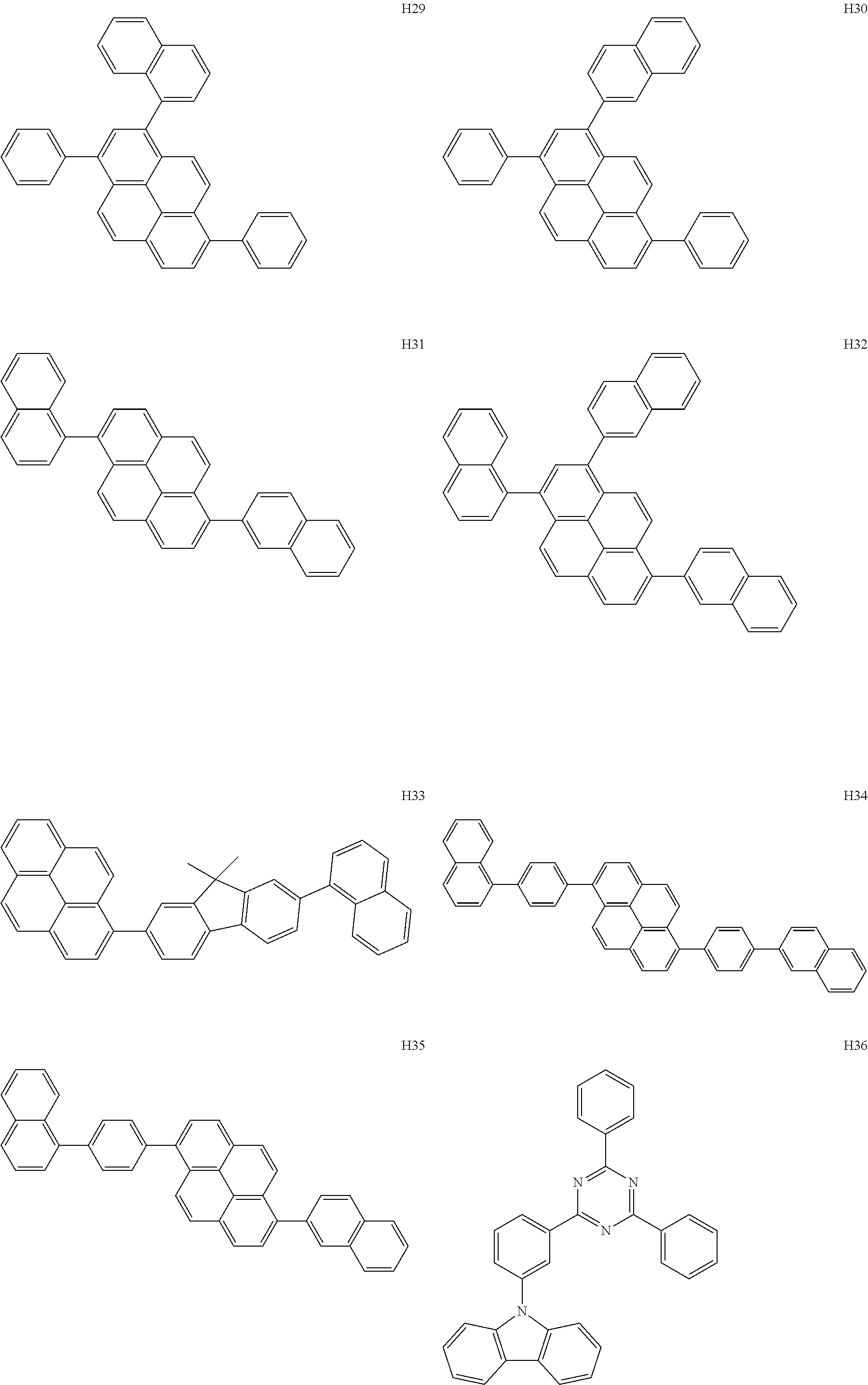

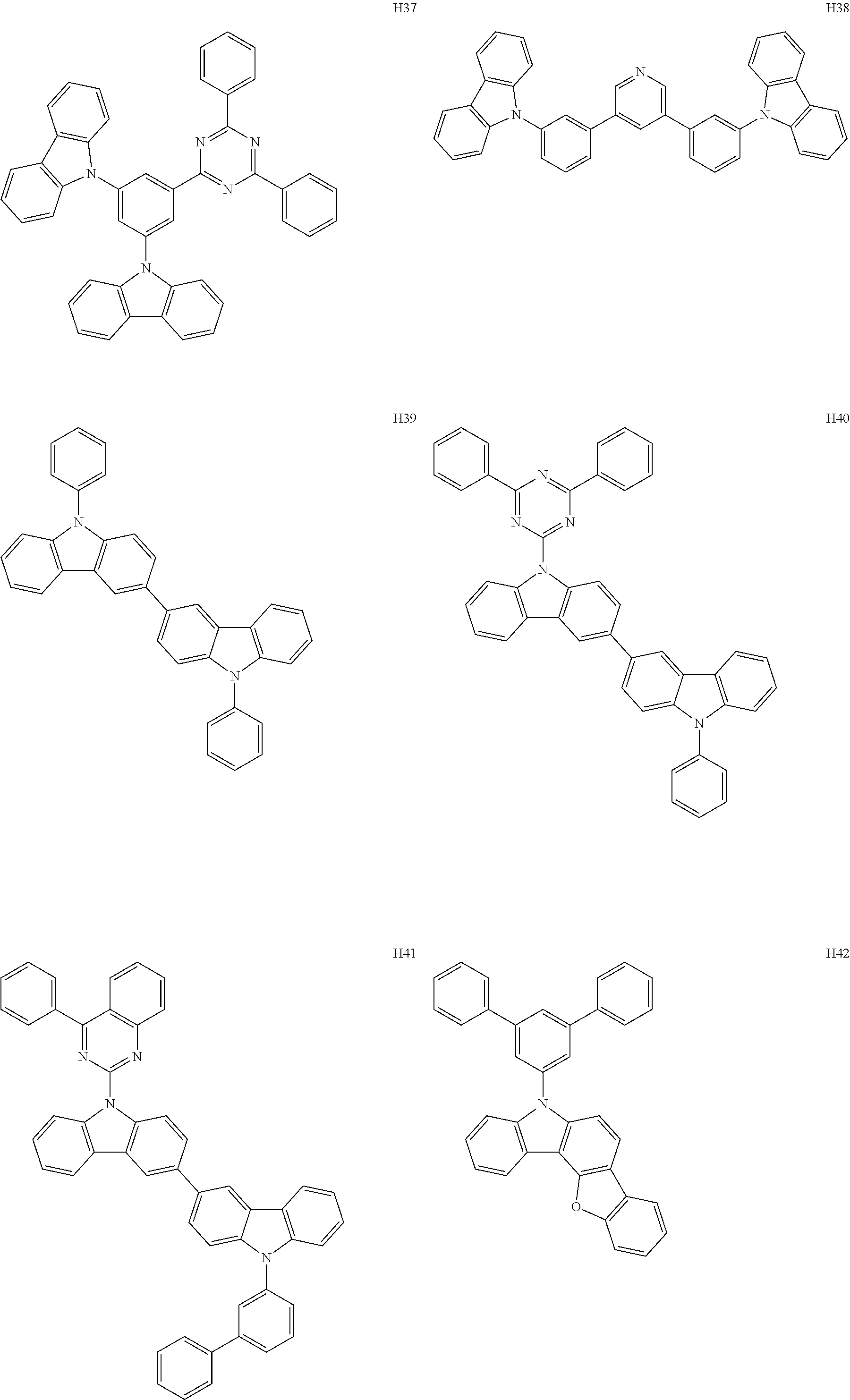 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

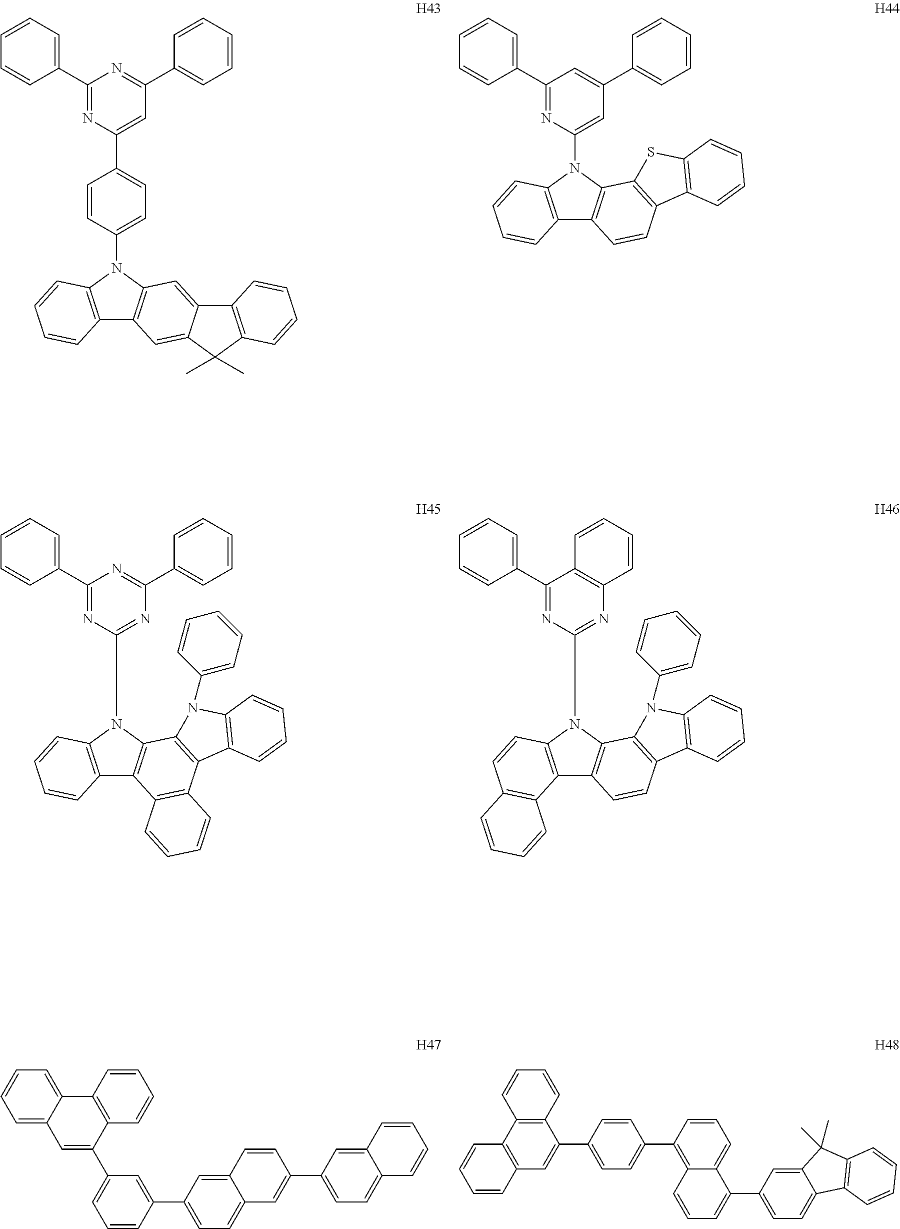

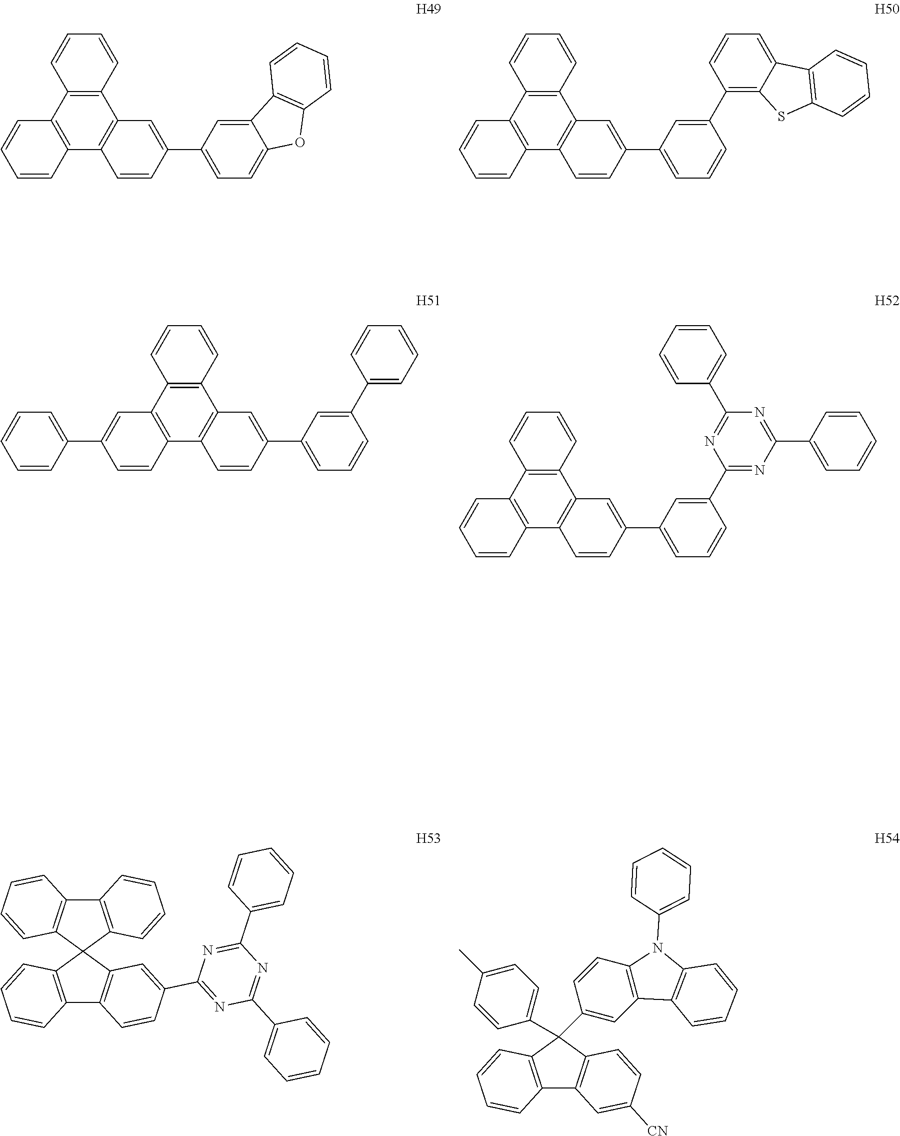

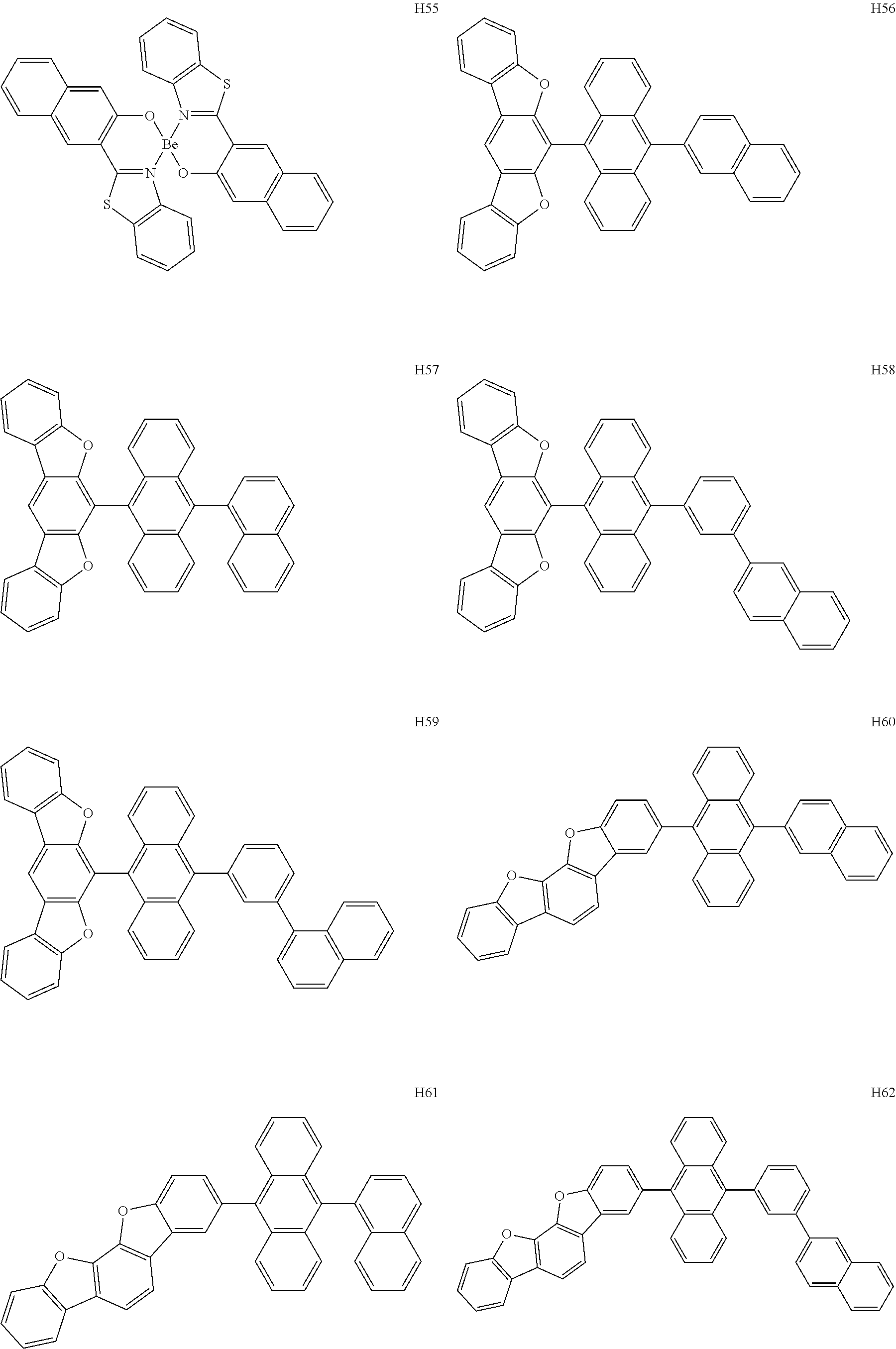

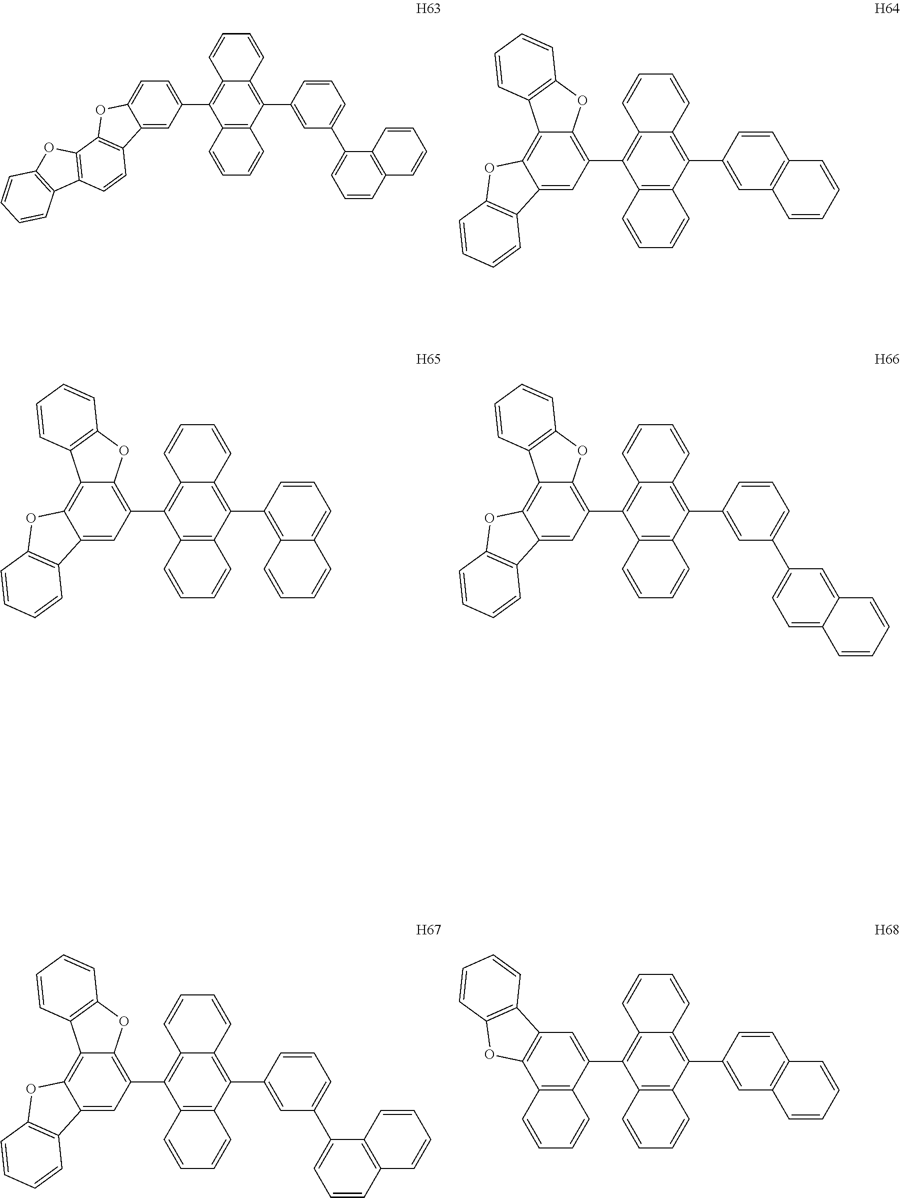 [SMILES: c1cc(-c2cccc3ccccc23)cc(-c2c3ccccc3c(-c3ccc4oc5c(ccc6c7ccccc7oc65)c4c3)c3ccccc23)c1 ; c1ccc2cc(-c3c4ccccc4c(-c4cc5c6ccccc6oc5c5c4oc4ccccc45)c4ccccc34)ccc2c1 ; c1ccc2c(-c3c4ccccc4c(-c4cc5c6ccccc6oc5c5c4oc4ccccc45)c4ccccc34)cccc2c1 ; c1cc(-c2ccc3ccccc3c2)cc(-c2c3ccccc3c(-c3cc4c5ccccc5oc4c4c3oc3ccccc34)c3ccccc23)c1 ; c1cc(-c2cccc3ccccc23)cc(-c2c3ccccc3c(-c3cc4c5ccccc5oc4c4c3oc3ccccc34)c3ccccc23)c1 ; c1ccc2cc(-c3c4ccccc4c(-c4cc5c6ccccc6oc5c5ccccc45)c4ccccc34)ccc2c1]

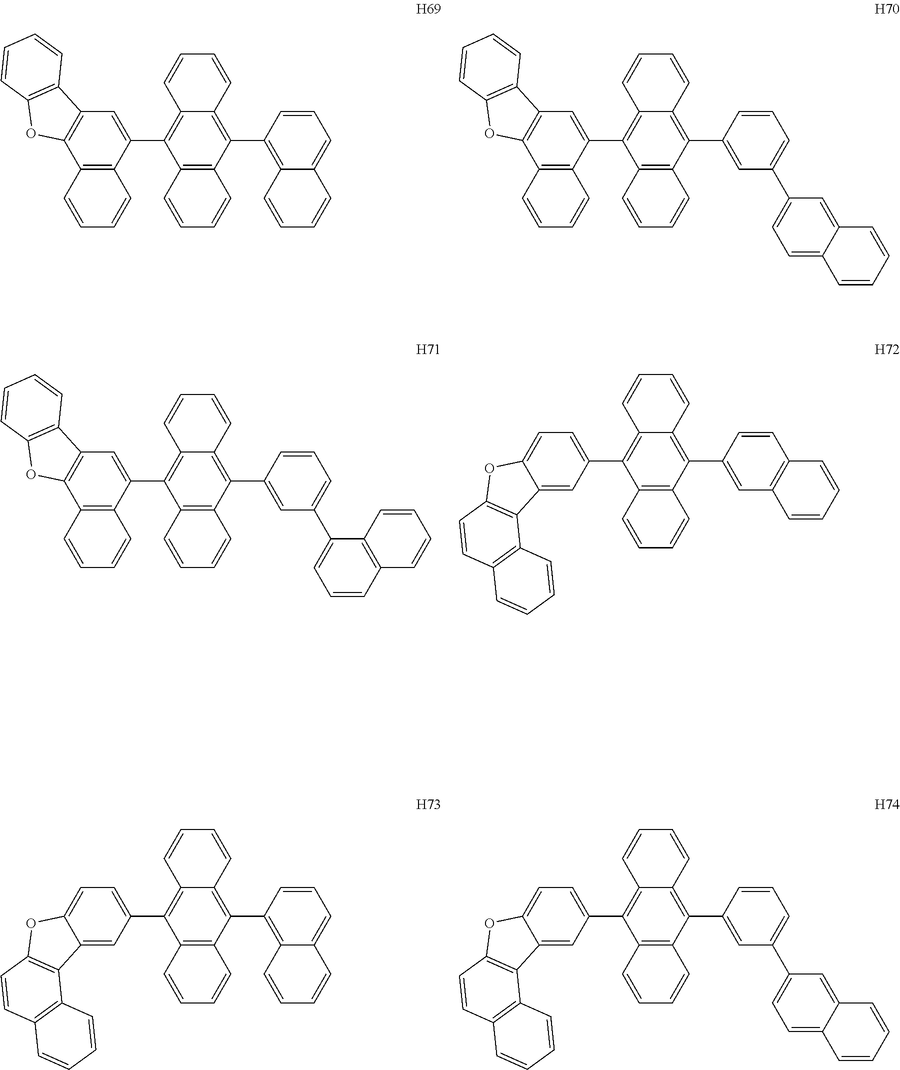

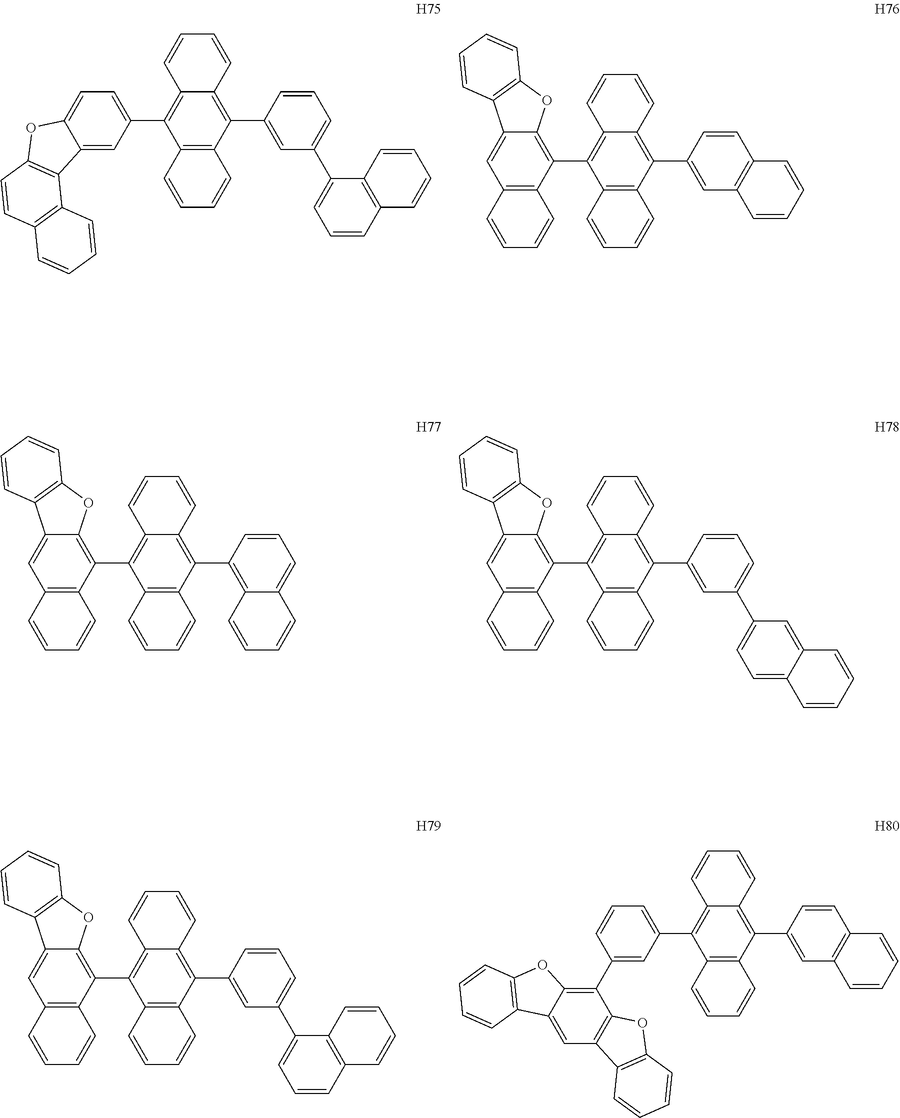 [SMILES: c1cc(-c2cccc3ccccc23)cc(-c2c3ccccc3c(-c3ccc4oc5ccc6ccccc6c5c4c3)c3ccccc23)c1 ; c1ccc2cc(-c3c4ccccc4c(-c4c5ccccc5cc5c4oc4ccccc45)c4ccccc34)ccc2c1 ; c1ccc2c(-c3c4ccccc4c(-c4c5ccccc5cc5c4oc4ccccc45)c4ccccc34)cccc2c1 ; c1cc(-c2ccc3ccccc3c2)cc(-c2c3ccccc3c(-c3c4ccccc4cc4c3oc3ccccc34)c3ccccc23)c1 ; c1cc(-c2cccc3ccccc23)cc(-c2c3ccccc3c(-c3c4ccccc4cc4c3oc3ccccc34)c3ccccc23)c1 ; c1cc(-c2c3ccccc3c(-c3ccc4ccccc4c3)c3ccccc23)cc(-c2c3oc4ccccc4c3cc3c2oc2ccccc23)c1]

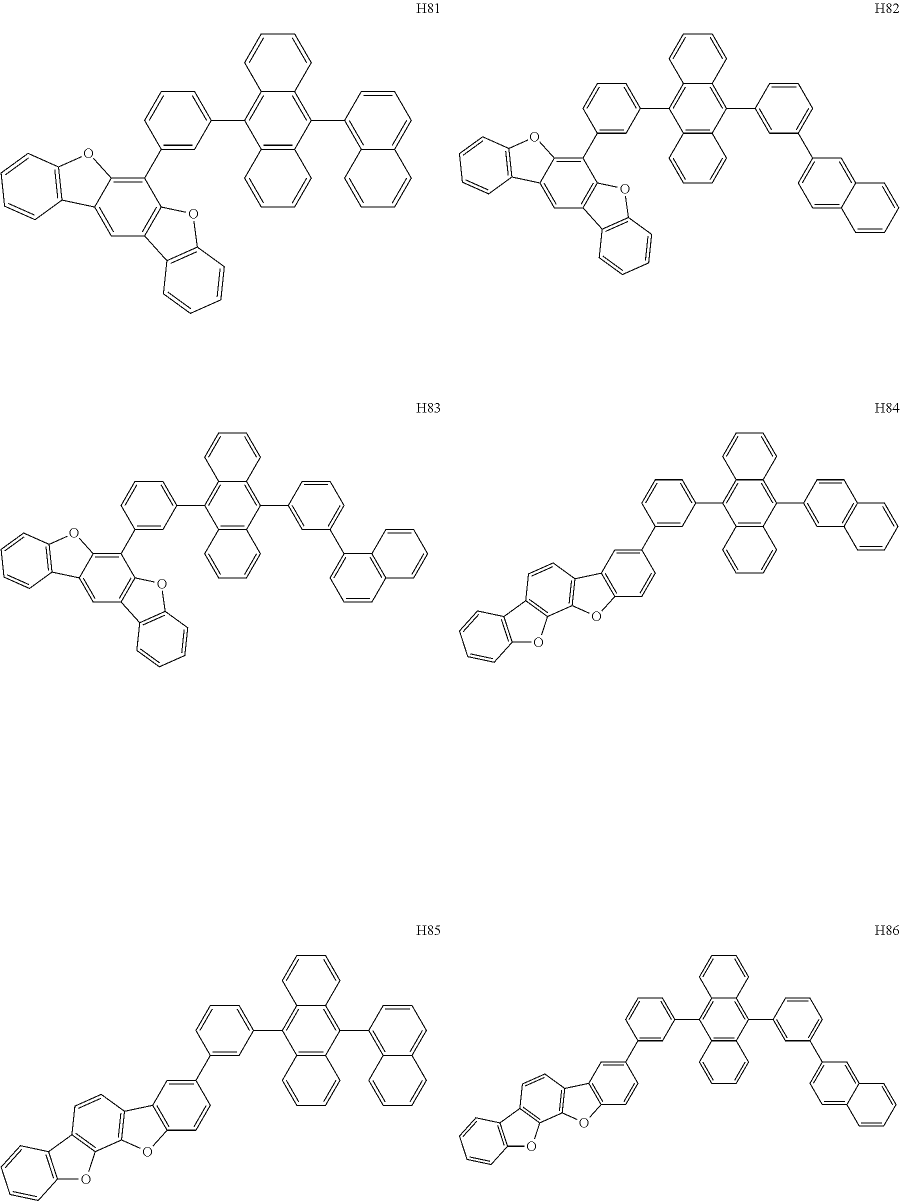

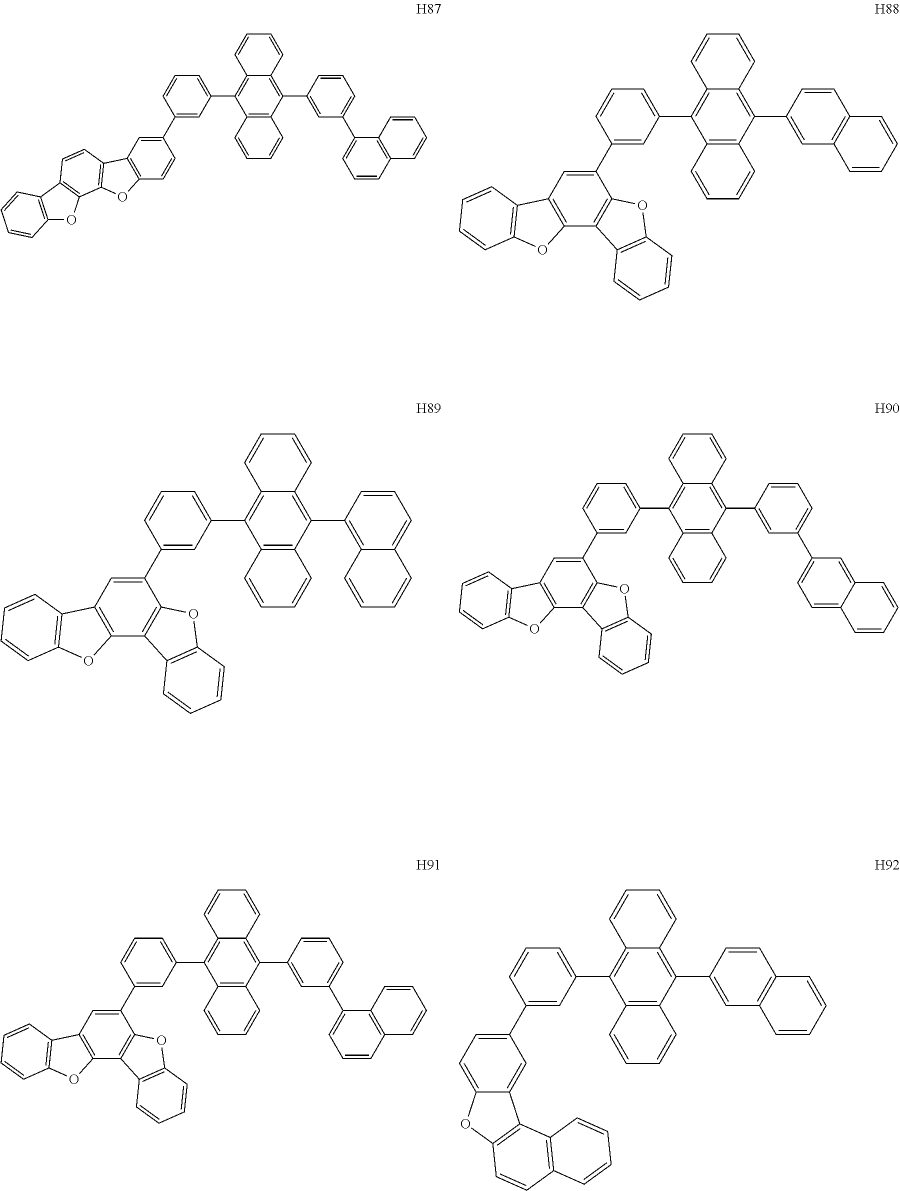

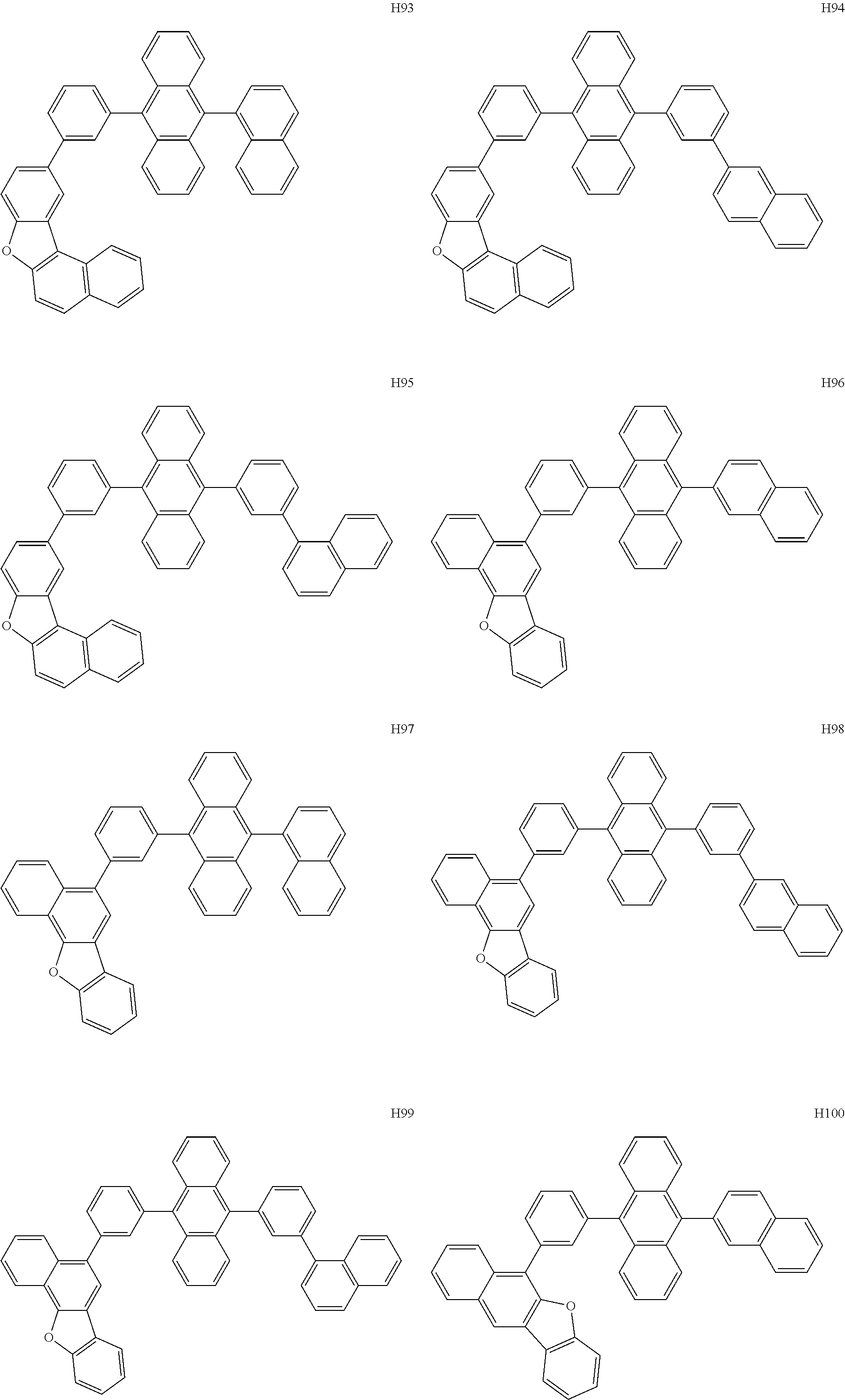

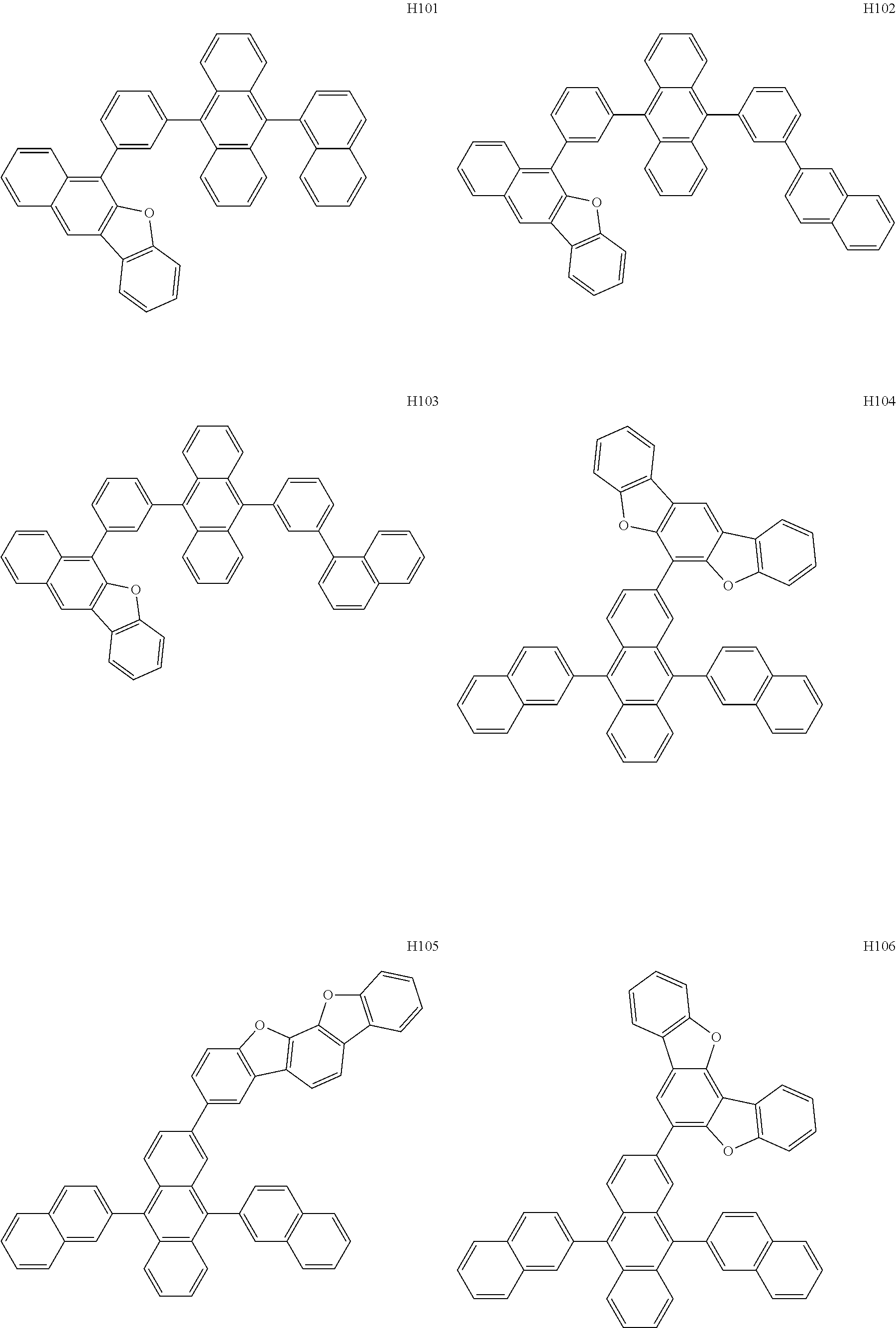

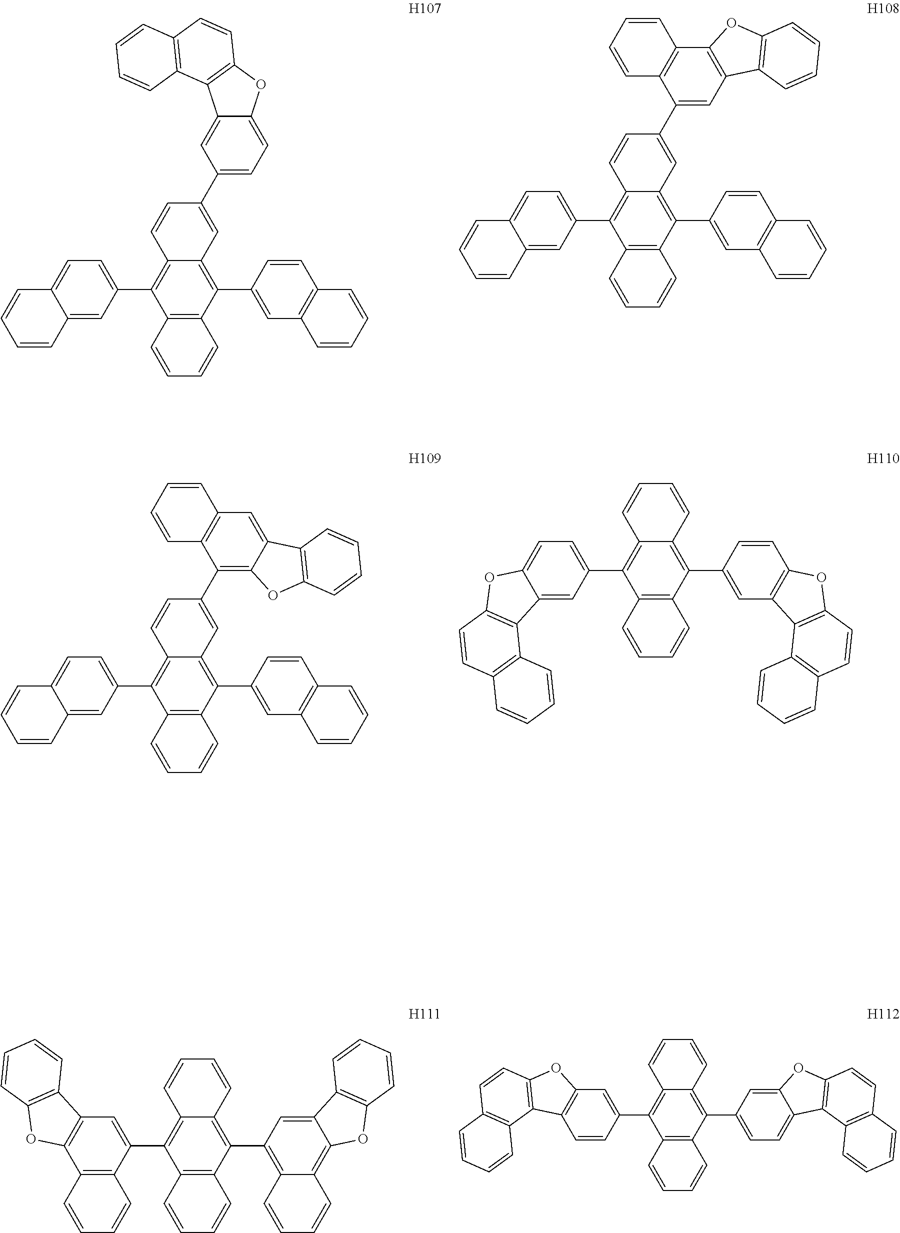 [SMILES: c1ccc2cc(-c3c4ccccc4c(-c4ccc5ccccc5c4)c4cc(-c5ccc6oc7ccc8ccccc8c7c6c5)ccc34)ccc2c1 ; c1ccc2cc(-c3c4ccccc4c(-c4ccc5ccccc5c4)c4cc(-c5cc6c7ccccc7oc6c6ccccc56)ccc34)ccc2c1 ; c1ccc2cc(-c3c4ccccc4c(-c4ccc5ccccc5c4)c4cc(-c5c6ccccc6cc6c5oc5ccccc56)ccc34)ccc2c1 ; c1ccc2c(c1)ccc1oc3ccc(-c4c5ccccc5c(-c5ccc6oc7ccc8ccccc8c7c6c5)c5ccccc45)cc3c12 ; c1ccc2c(c1)oc1c3ccccc3c(-c3c4ccccc4c(-c4cc5c6ccccc6oc5c5ccccc45)c4ccccc34)cc21 ; c1ccc2c(c1)ccc1oc3cc(-c4c5ccccc5c(-c5ccc6c(c5)oc5ccc7ccccc7c56)c5ccccc45)ccc3c12]

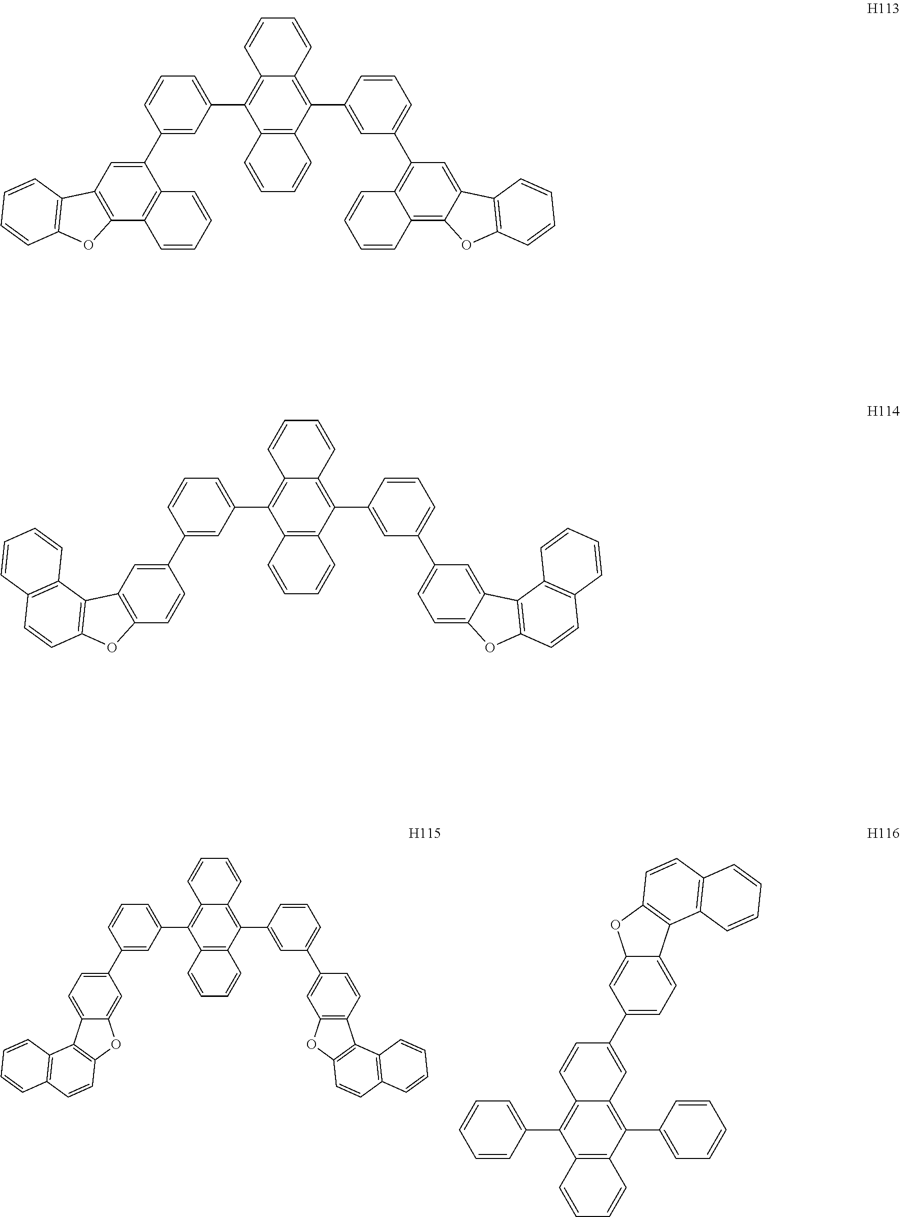 [SMILES: c1cc(-c2c3ccccc3c(-c3cccc(-c4cc5c6ccccc6oc5c5ccccc45)c3)c3ccccc23)cc(-c2cc3c4ccccc4oc3c3ccccc23)c1 ; c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1 ; c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1]

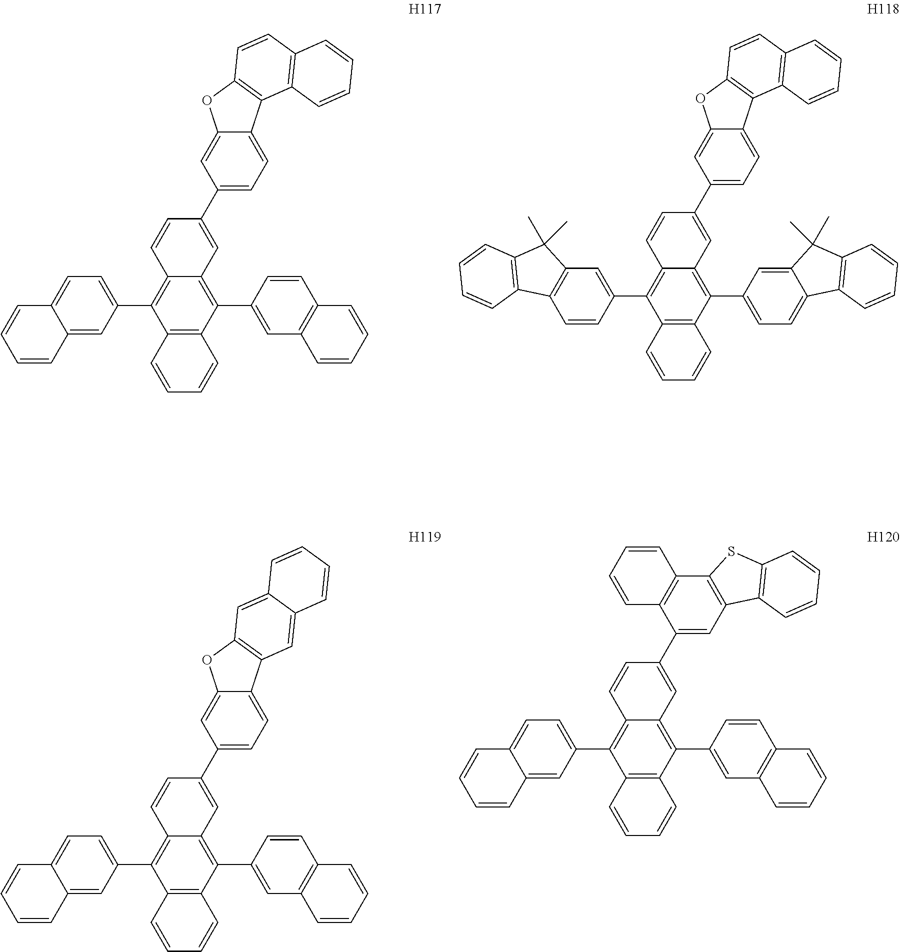 [SMILES: c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21 ; c1ccc2cc(-c3c4ccccc4c(-c4ccc5ccccc5c4)c4cc(-c5ccc6c(c5)oc5cc7ccccc7cc56)ccc34)ccc2c1 ; c1ccc2cc(-c3c4ccccc4c(-c4ccc5ccccc5c4)c4cc(-c5cc6c7ccccc7sc6c6ccccc56)ccc34)ccc2c1]

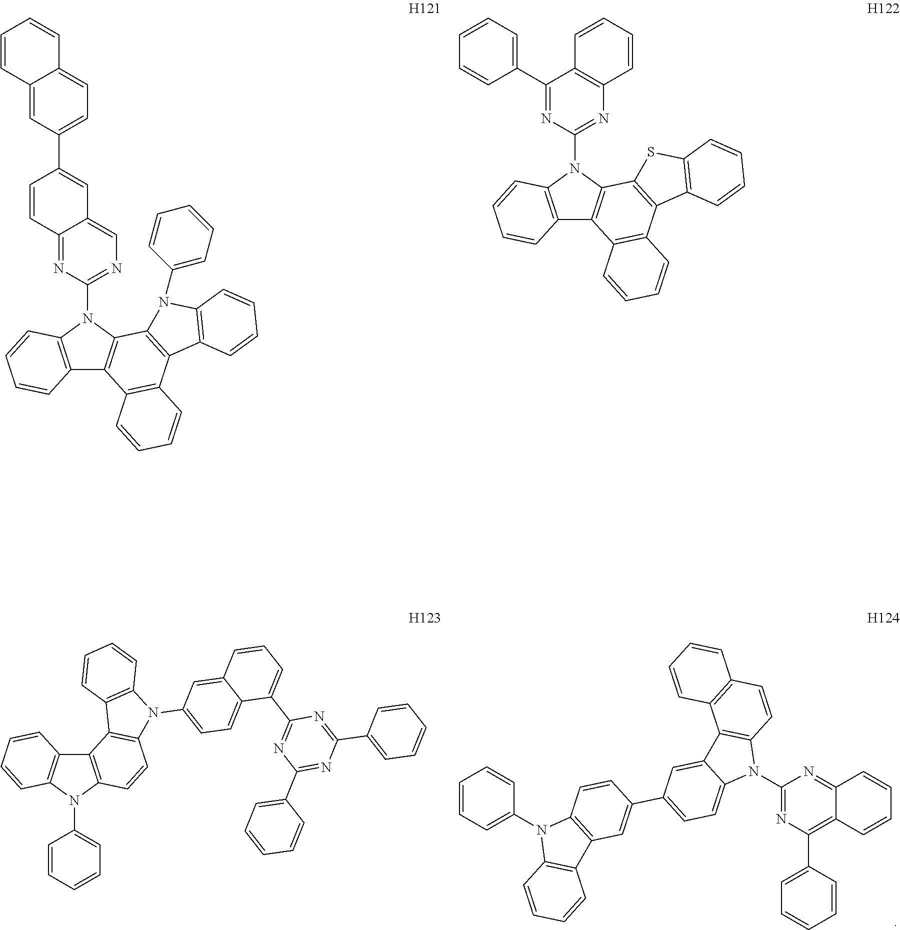 [SMILES: c1ccc(-n2c3ccccc3c3c4ccccc4c4c5ccccc5n(-c5ncc6cc(-c7ccc8ccccc8c7)ccc6n5)c4c32)cc1 ; c1ccc(-c2nc(-n3c4ccccc4c4c5ccccc5c5c6ccccc6sc5c43)nc3ccccc23)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

[Phosphorescent Dopant]

In one or more embodiments, the phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

For example, the phosphorescent dopant may include an organometallic compound represented by Formula 401:

M(L₄₀₁)_{xc1}(L₄₀₂)_{xc2} Formula 401:

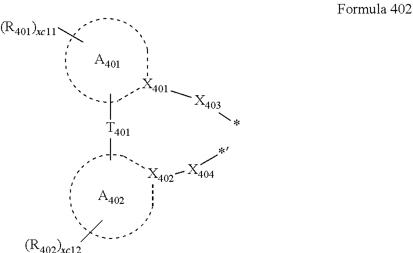

wherein, in Formulas 401 and 402,
M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein when xc1 is two or more, two or more of L₄₀₁(s) may be identical to or different from each other,
L₄₀₂ may be an organic ligand, and xc2 may be 0, 1, 2, 3, or 4, and when xc2 is 2 or more, two or more of L₄₀₂(s) may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)-*′, *—C(Q₄₁₁)(Q₄₁₂)-*′, *—C(Q₄₁₁)═C(Q₄₁₂)-*′, *—C(Q₄₁₁)=*′, or *═C(Q₄₁₁)=*′,
X₄₀₃ and X₄₀₄ may each independently be a chemical bond (for example, a covalent bond or a coordination bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ may respectively be the same as described in connection with Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may respectively be the same as described in connection with Q₁,
xc11 and xc12 may each independently be an integer from 0 to 10, and
* and *′ in Formula 402 each indicate a binding site to M in Formula 401.

For example, in Formula 402, i) X₄₀₁ may be nitrogen, and X₄₀₂ may be carbon, or ii) each of X₄₀₁ and X₄₀₂ may be nitrogen.

In one or more embodiments, when xc1 in Formula 402 is 2 or more, two ring A₄₀₁ in two or more of L₄₀₁(s) may be optionally linked to each other via T₄₀₂, which is a linking group, and two ring A₄₀₂ may be optionally linked to each other via T₄₀₃, which is a linking group (see Compounds PD1 to PD4 and PD7). T₄₀₂ and T₄₀₃ may respectively be the same as described in connection with T₄₀₁.

In Formula 401, L₄₀₂ may be an organic ligand. For example, L₄₀₂ may include a halogen group, a diketone group (for example, an acetylacetonate group), a carboxylic acid group (for example, a picolinate group), —C(═O), an isonitrile group, —CN group, a phosphorus group (for example, a phosphine group, a phosphite group, and the like), or any combination thereof.

The phosphorescent dopant may include, for example, one of compounds PD1 to PD25, or any combination thereof:

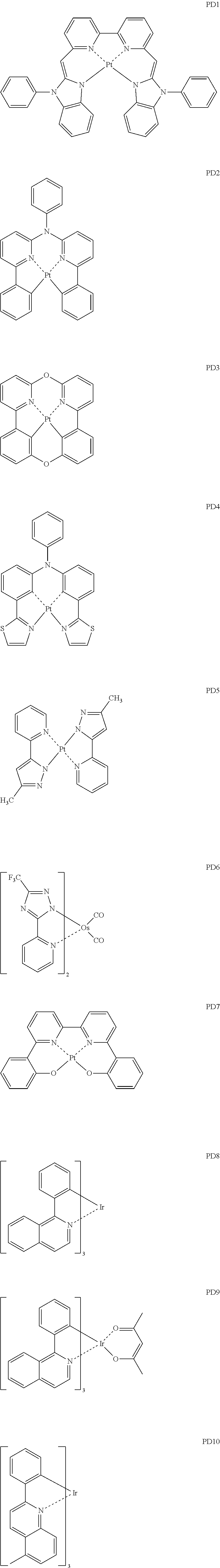

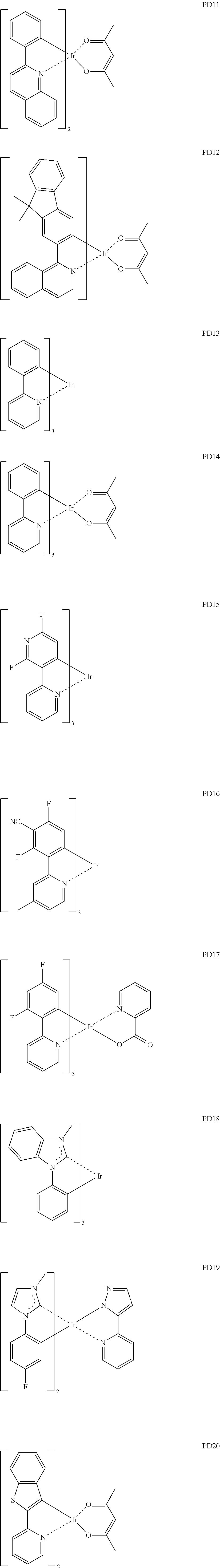

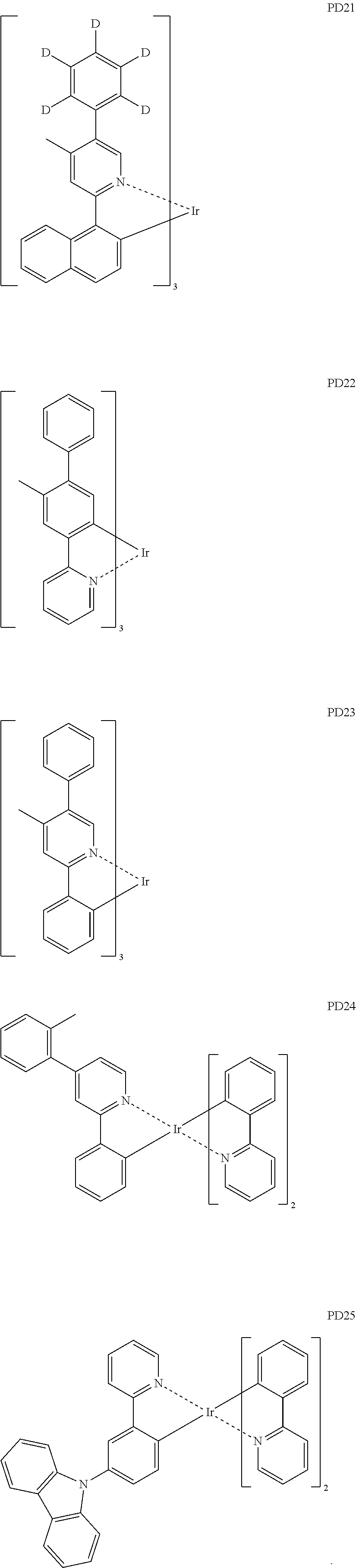

[Fluorescent Dopant]

The fluorescent dopant may include an amine group-containing compound, a styryl group-containing compound, or any combination thereof.

For example, the fluorescent dopant may include a compound represented by Formula 501:

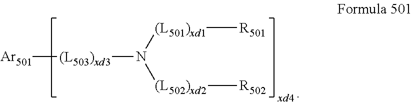

In Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

For example, Ar₅₀₁ in Formula 501 may be a condensed cyclic group (for example, an anthracene group, a chrysene group, or a pyrene group) in which three or more monocyclic groups are condensed together.

In one or more embodiments, xd4 in Formula 501 may be 2.

For example, the fluorescent dopant may include: one of Compounds FD1 to FD36; DPVBi; DPAVBi; or any combination thereof:

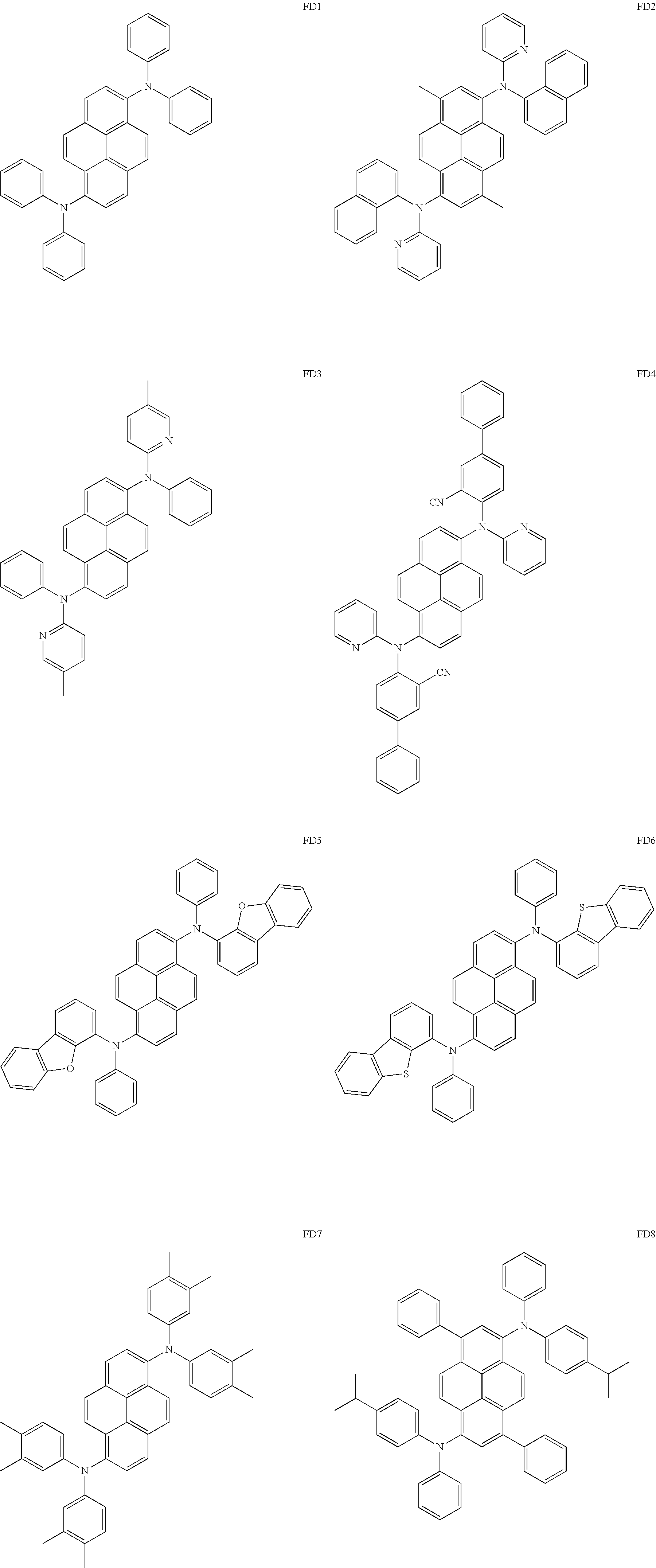

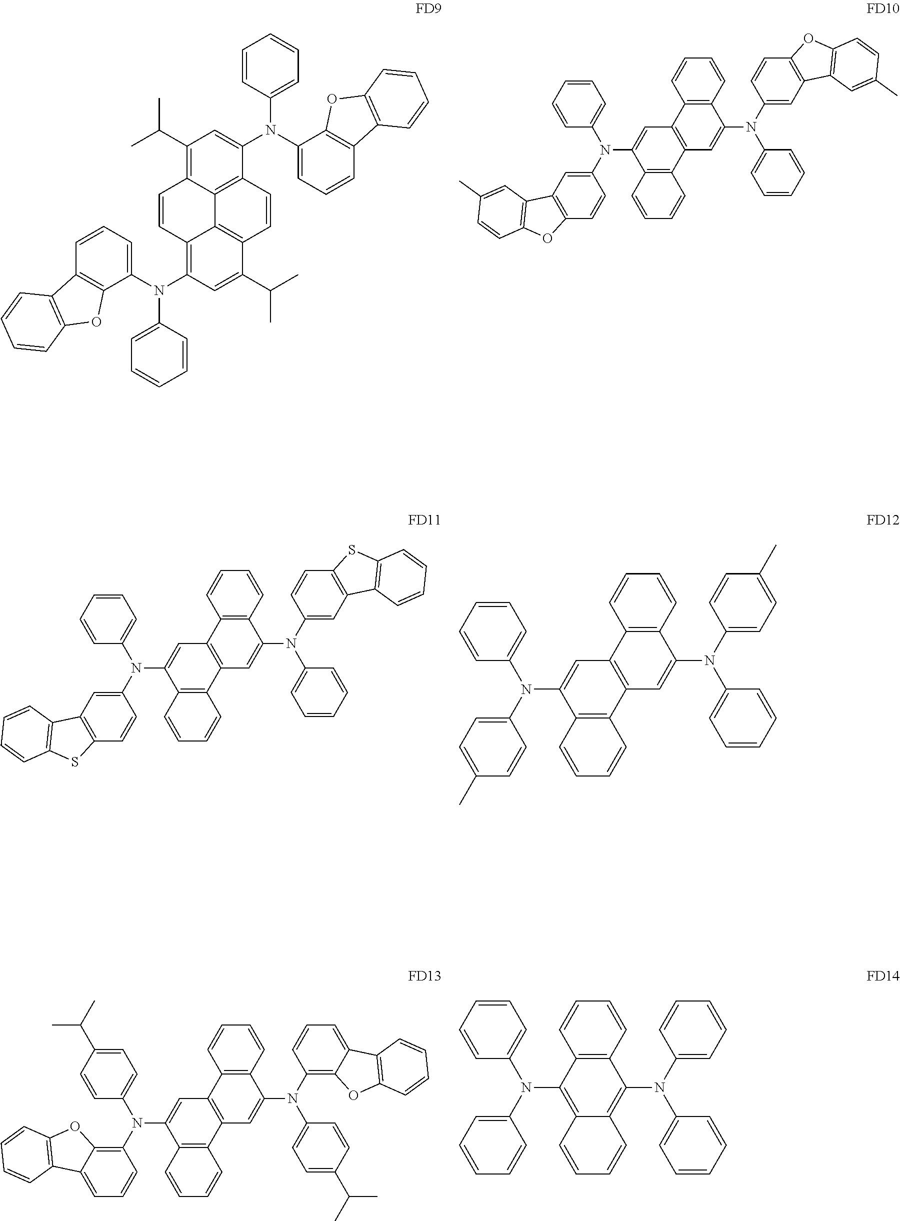

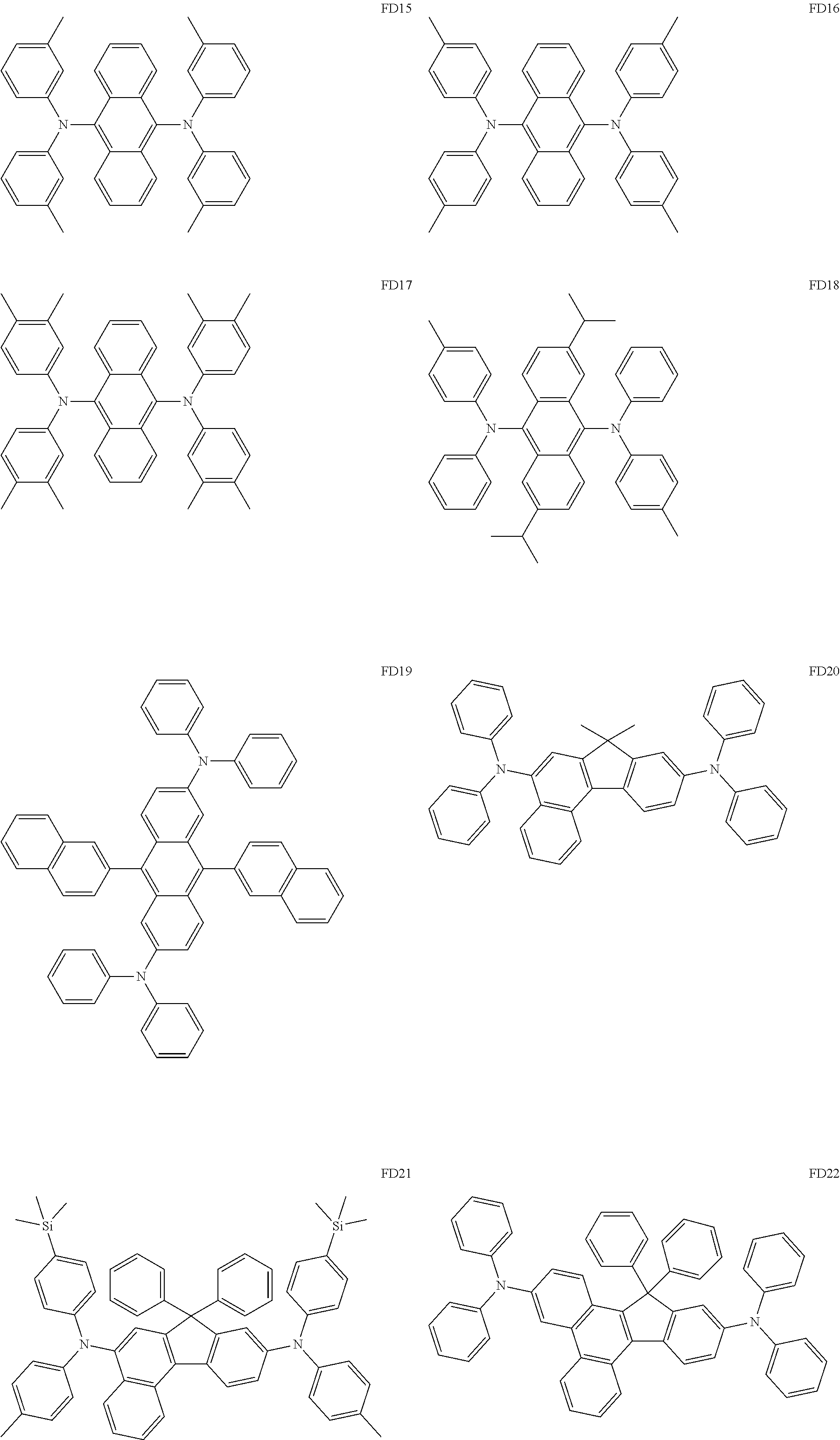

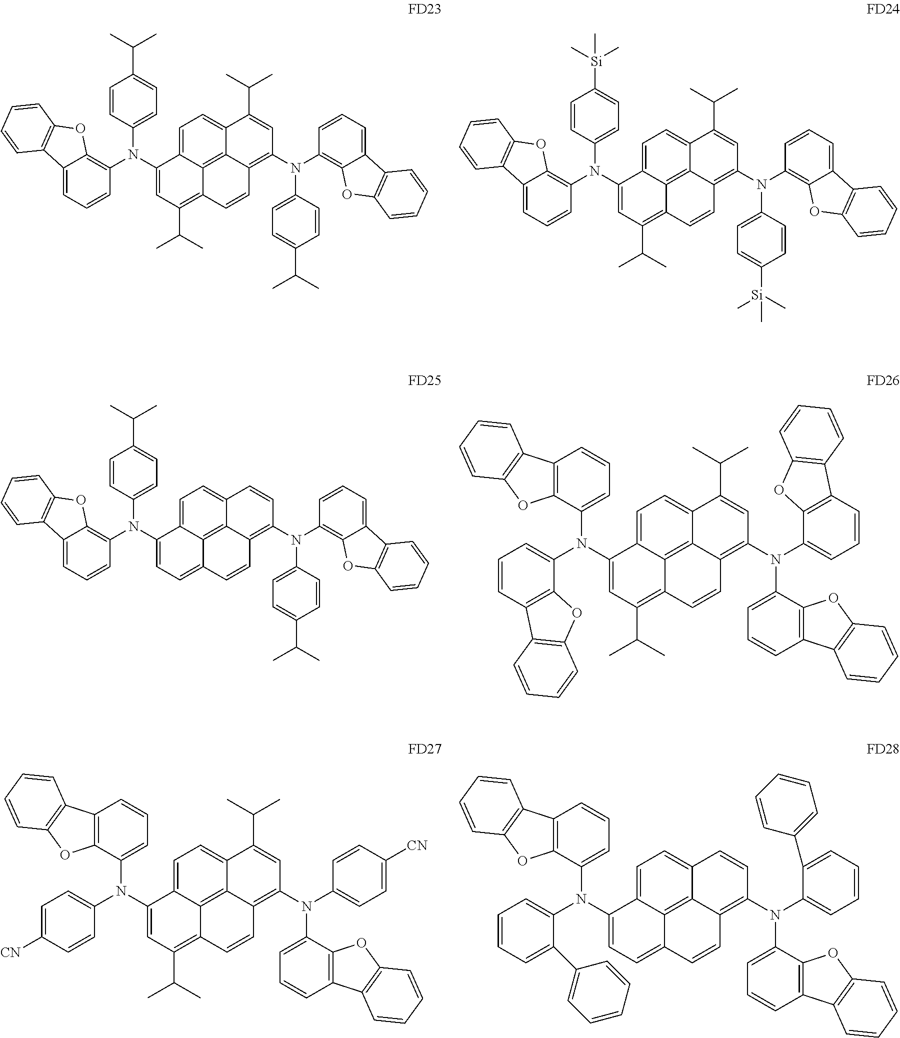

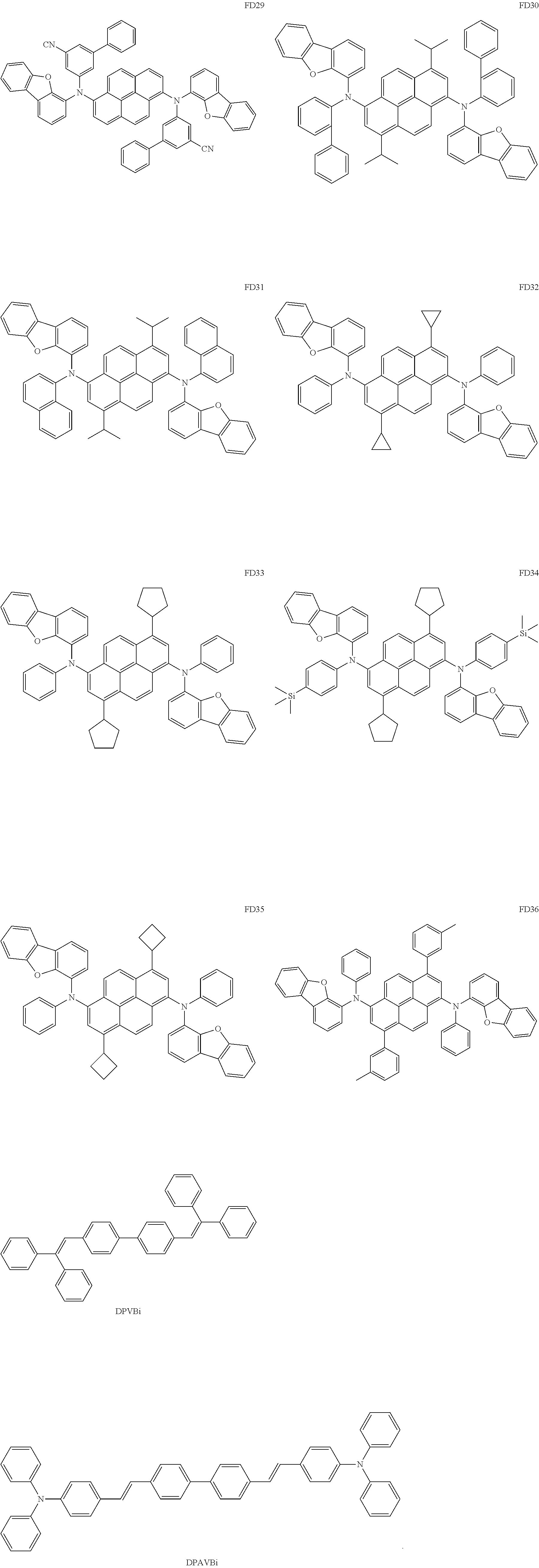

[Delayed Fluorescence Material]

The emission layer may include a delayed fluorescence material.

In the embodiments described herein, the delayed fluorescence material may be selected from compounds capable of emitting delayed fluorescent light based on a delayed fluorescence emission mechanism.

The delayed fluorescence material included in the emission layer may act as a host or a dopant depending on the type of other materials included in the emission layer.

In one or more embodiments, the difference between the triplet energy level (eV) of the delayed fluorescence material and the singlet energy level (eV) of the delayed fluorescence material may be greater than or equal to 0 eV and less than or equal to 0.5 eV. When the difference between the triplet energy level (eV) of the delayed fluorescence material and the singlet energy level (eV) of the delayed fluorescence material satisfies the above-described range, up-conversion from the triplet state to the singlet state of the delayed fluorescence materials may effectively occur, and thus, the luminescence efficiency of the light-emitting device 10 may be improved.

For example, the delayed fluorescence material may include i) a material including at least one electron donor (for example, a π electron-rich C₃-C₆₀ cyclic group, such as a carbazole group) and at least one electron acceptor (for example, a sulfoxide group, a cyano group, or a π electron-deficient nitrogen-containing C₁-C₆₀ cyclic group), and ii) a material including a C₈-C₆₀ polycyclic group in which two or more cyclic groups are condensed while sharing boron (B).

Examples of the delayed fluorescence material may include at least one of the following compounds DF1 to DF9:

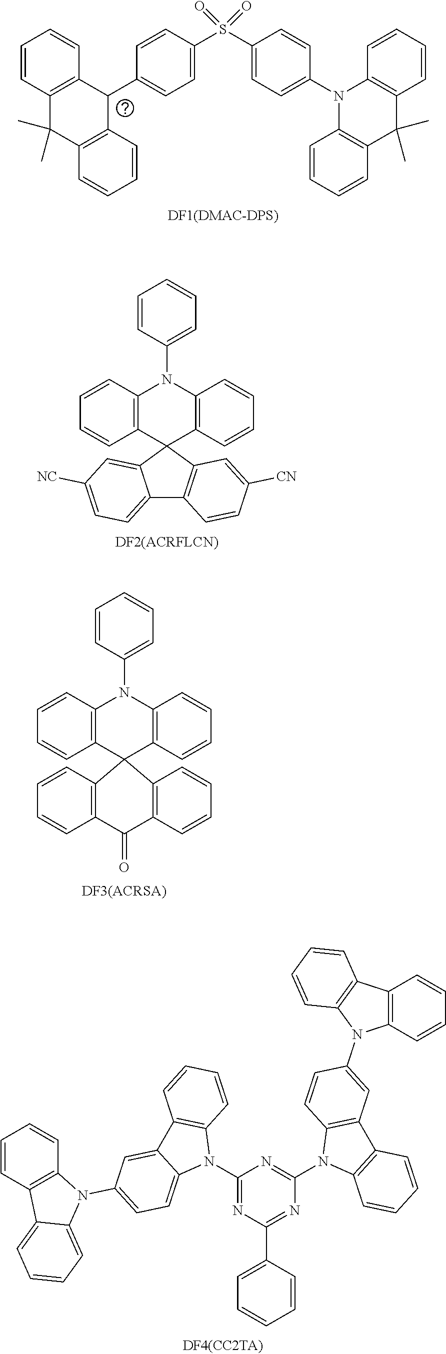

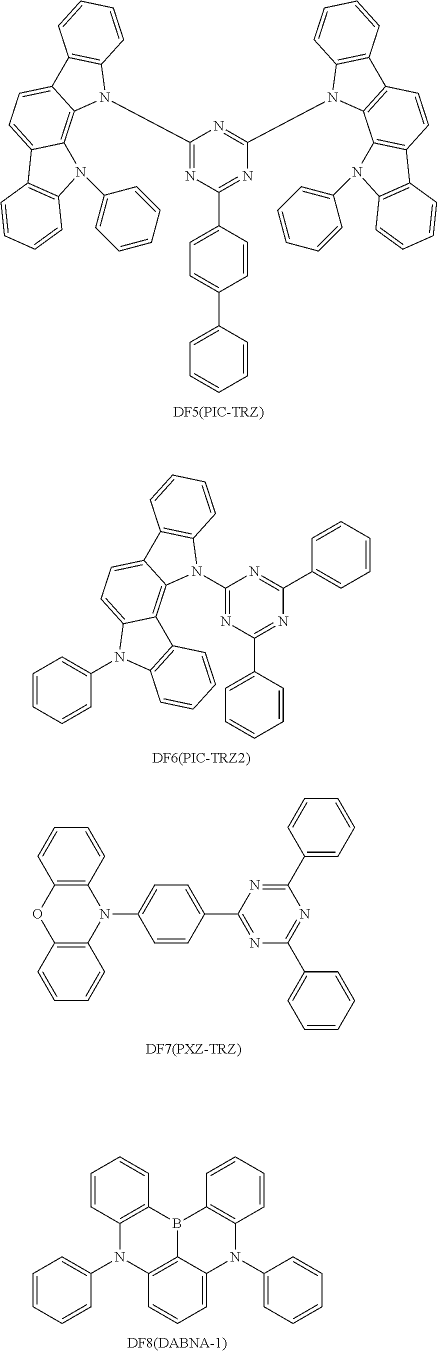 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccc(N4c5ccccc5Oc5ccccc54)cc3)n2)cc1 ; c1ccc(N2c3ccccc3B3c4ccccc4N(c4ccccc4)c4cccc2c43)cc1]

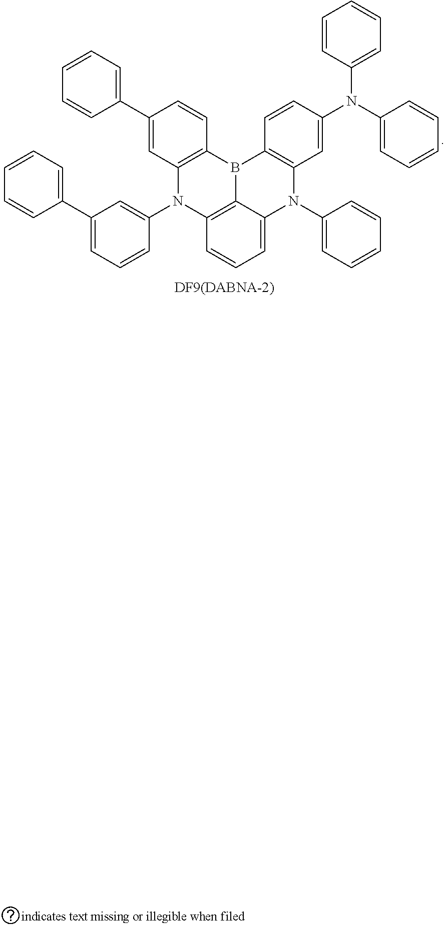 [SMILES: c1ccc(-c2cccc(N3c4cc(-c5ccccc5)ccc4B4c5ccc(N(c6ccccc6)c6ccccc6)cc5N(c5ccccc5)c5cccc3c54)c2)cc1]

[Quantum Dot]

The emission layer may include a quantum dot.

The term “quantum dot” as used herein refers to a crystal of a semiconductor compound, and may include any material capable of emitting light of various emission wavelengths according to the size of the crystal.

A diameter of the quantum dot may be, for example, in a range of about 1 nm to about 10 nm.

The quantum dot may be synthesized by a wet chemical process, a metal organic chemical vapor deposition process, a molecular beam epitaxy process, or any process similar thereto.

The wet chemical process is a method including mixing a precursor material with an organic solvent and then growing a quantum dot particle crystal. When the crystal grows, the organic solvent naturally acts as a dispersant coordinated on the surface of the quantum dot crystal and controls the growth of the crystal so that the growth of quantum dot particles can be controlled through a process which costs lower, and is easier than vapor deposition methods, such as metal organic chemical vapor deposition (MOCVD) or molecular beam epitaxy (MBE),

The quantum dot may include Group II-VI semiconductor compounds, Group III-V semiconductor compounds, Group III-VI semiconductor compounds, Group 1-III-VI semiconductor compounds, Group IV-VI semiconductor compounds, a Group IV element or compound, or any combination thereof.

Examples of the Group II-VI semiconductor compound are: a binary compound, such as CdSe, CdTe, ZnS, ZnSe, ZnTe, ZnO, HgS, HgSe, HgTe, MgSe, or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, or MgZnS; a quaternary compound, such as CdZnSeS, CdZnSeTe, CdZnSTe, CdHgSeS, CdHgSeTe, CdHgSTe, HgZnSeS, HgZnSeTe, or HgZnSTe; or any combination thereof.

Examples of the Group III-V semiconductor compound are: a binary compound, such as GaN, GaP, GaAs, GaSb, AlN, AlP, AlAs, AlSb, InN, InP, InAs, InSb, or the like; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, InPSb, GaAlNP, or the like; a quaternary compound, such as GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, or the like; or any combination thereof. The Group III-V semiconductor compound may further include a Group II element. Examples of the Groups III-V semiconductor compound further including a Group II element are InZnP, InGaZnP, InAlZnP, and the like.

Examples of the Group III-VI semiconductor compound are: a binary compound, such as GaS, GaSe, Ga₂Se₃, GaTe, InS, InSe, In₂S₃, In₂Se₃, or InTe; a ternary compound, such as InGaS₃, or InGaSe₃; and any combination thereof.

Examples of the Group I-III-VI semiconductor compound are: a ternary compound, such as AgInS, AgInS₂, CuInS, CuInS₂, CuGaO₂, AgGaO₂, or AgAlO₂; or any combination thereof.

Examples of the Group IV-VI semiconductor compound are: a binary compound, such as SnS, SnSe, SnTe, PbS, PbSe, or PbTe; a ternary compound, such as SnSeS, SnSeTe, SnSTe, PbSeS, PbSeTe, PbSTe, SnPbS, SnPbSe, or SnPbTe; a quaternary compound, such as SnPbSSe, SnPbSeTe, or SnPbSTe; or any combination thereof.

The Group IV element or compound may include: a single element compound, such as Si or Ge; a binary compound, such as SiC or SiGe; or any combination thereof.

Each element included in a multi-element compound such as the binary compound, the ternary compound, and the quaternary compound may be present at a uniform concentration or non-uniform concentration in a particle.

The quantum dot may have a single structure in which the concentration of each element in the quantum dot is uniform, or a core-shell dual structure. For example, the material included in the core and the material included in the shell may be different from each other.

The shell of the quantum dot may act as a protective layer that prevents chemical degeneration of the core to maintain semiconductor characteristics, and/or as a charging layer that imparts electrophoretic characteristics to the quantum dot. The shell may be a single layer or a multi-layer. The interface between the core and the shell may have a concentration gradient in which the concentration of an element existing in the shell decreases toward the center of the core.

Examples of the shell of the quantum dot may be an oxide of metal, metalloid, or non-metal, a semiconductor compound, and any combination thereof. Examples of the oxide of metal, metalloid, or non-metal are: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO; a ternary compound, such as MgAl₂O₄, CoFe₂O₄, NiFe₂O₄, or CoMn₂O₄; and any combination thereof. Examples of the semiconductor compound are: as described herein, a Group II-VI semiconductor compound; a Group III-V semiconductor compound; a Group III-VI semiconductor compound; a Group I-III-VI semiconductor compound; a Group IV-VI semiconductor compound; or any combination thereof. For example, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of the emission wavelength spectrum of the quantum dot may be about 45 nm or less, for example, about 40 nm or less, for example, about 30 nm or less, and within these ranges, color purity or color reproducibility may be increased. In addition, since the light emitted through the quantum dot is emitted in all directions, the wide viewing angle may be improved.

In addition, the quantum dot may be in the form of a spherical particle, a pyramidal particle, a multi-arm particle, a cubic nanoparticle, a nanotube particle, a nanowire particle, a nanofiber particle, or a nanoplate particle.

Since the energy band gap may be adjusted by controlling the size of the quantum dot, light having various wavelength bands may be obtained from the quantum dot emission layer. Accordingly, by using quantum dots of different sizes, a light-emitting device that emits light of various wavelengths may be implemented. In one or more embodiments, the size of the quantum dot may be selected to emit red, green and/or blue light. In addition, the size of the quantum dot may be configured to emit white light by combination of light of various colors.

Electron Transport Region in Interlayer 130

The electron transport region may have: i) a single-layered structure consisting of a single layer consisting of a single material, ii) a single-layered structure consisting of a single layer consisting of a plurality of different materials, or iii) a multi-layered structure including a plurality of layers including different materials.

The electron-transporting region may include a buffer layer, a hole-blocking layer, an electron control layer, an electron-transporting layer, an electron injection layer, or any combination thereof.

For example, the electron transport region may have an electron transport layer/electron injection layer structure, a hole-blocking layer/electron transport layer/electron injection layer structure, an electron control layer/electron transport layer/electron injection layer structure, or a buffer layer/electron transport layer/electron injection layer structure, wherein constituting layers of each structure are stacked from an emission layer.

In an embodiment, the electron transport region (for example, the buffer layer, the hole-blocking layer, the electron control layer, or the electron transport layer in the electron transport region) may include a metal-free compound including at least one π electron-deficient nitrogen-containing C₁-C₆₀ cyclic group.

For example, the electron transport region may include a compound represented by Formula 601:

[Ar₆₀₁]ₓₑ₁₁-[(L₆₀₁)ₓₑ₁-R₆₀₁]ₓₑ₂₁ Formula 601
wherein, in Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may respectively be the same as described in connection with Q₁, xe21 may be 1, 2, 3, 4, or 5, and
at least one of Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, when xe11 in Formula 601 is 2 or more, two or more of Ar₆₀₁(s) may be linked to each other via a single bond.

In other embodiments, Ar₆₀₁ in Formula 601 may be a substituted or unsubstituted anthracene group.

In other embodiments, the electron transport region may include a compound represented by Formula 601-1:

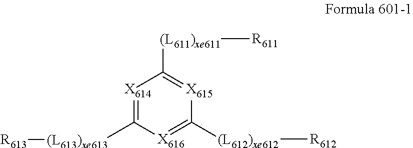

wherein, in Formula 601-1,
X₆₁₄ may be N or C(R₆₁₄), X₆₁₅ may be N or C(R₁₁₅), X₆₁₆ may be N or C(R₆₁₆), and at least one of X₆₁₄ to X₆₁₆ may be N,
L₆₁₁ to L₆₁₃ may respectively be the same as described in connection with L₆₀₁,
xe611 to xe613 may respectively be the same as described in connection with xe1,
R₆₁₁ to R₆₁₃ may respectively be the same as described in connection with R₆₀₁, and R₆₁₄ to R₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulas 601 and 601-1 may each independently be 0, 1, or 2.

The electron transport region may include one of Compound ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, diphenyl[4-(triphenylsilyl)phenyl]phosphine oxide (TSPO1), 2,2′,2″-(1,3,5-benzinetriyl)-tris(1-phenyl-1-H-benzimidazole) (TPBI), 2,4,6-Tris(3-(pyrimidin-5-yl)phenyl)-1,3,5-triazine (TPM-TAZ), or any combination thereof:

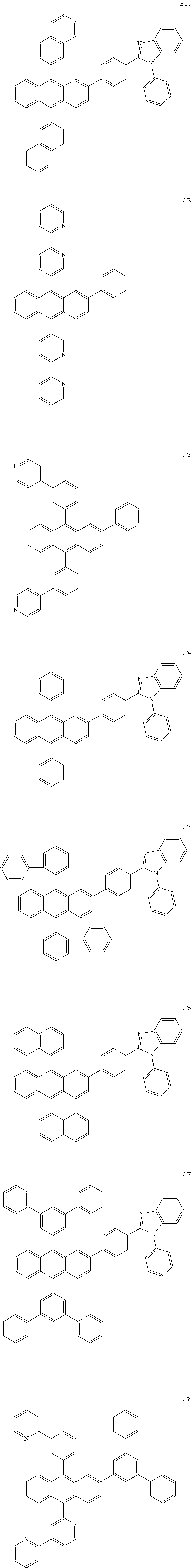

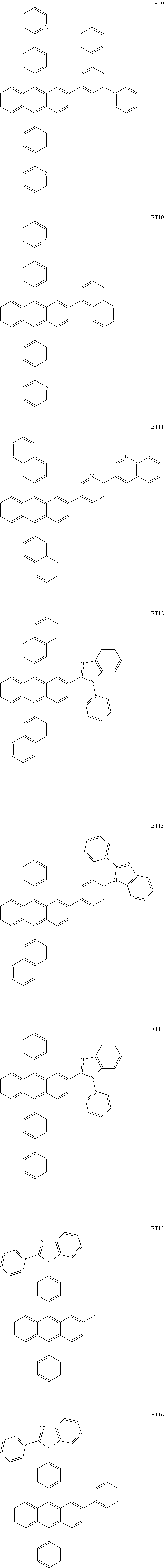

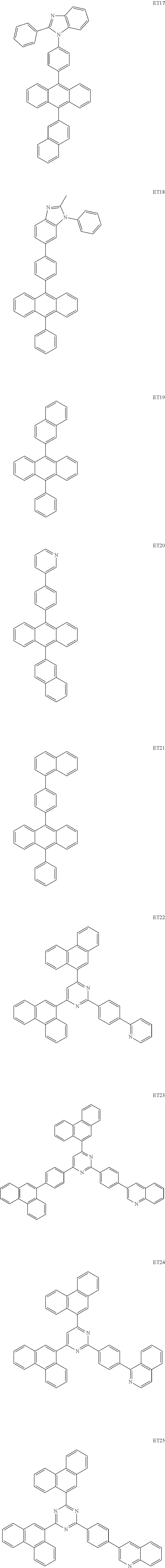

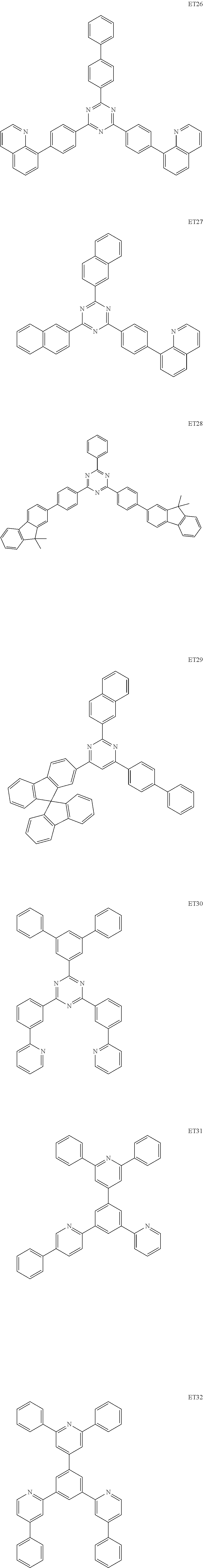

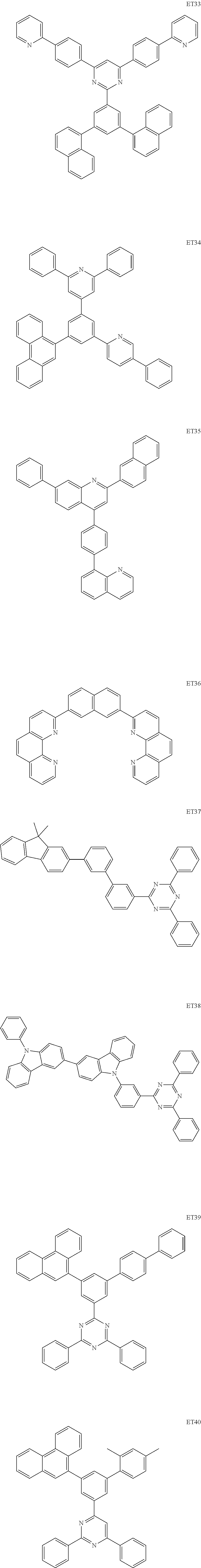

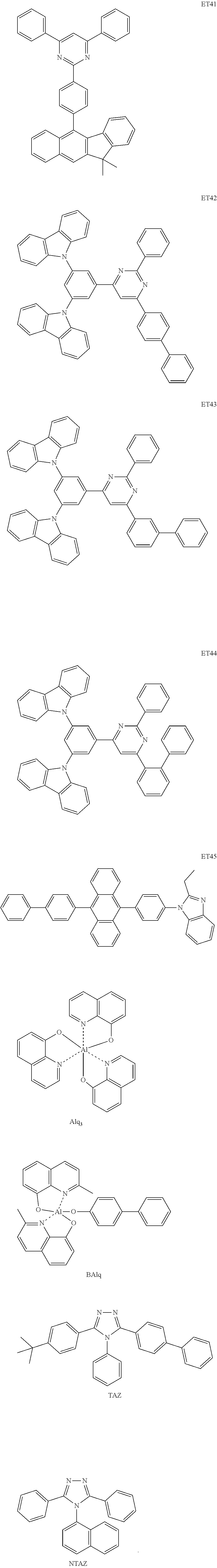

A thickness of the electron transport region may be from about 160 Å to about 5,000 Å, for example, from about 100 Å to about 4,000 Å. When the electron transport region includes a buffer layer, a hole-blocking layer, an electron control layer, an electron transport layer, or any combination thereof, the thickness of the buffer layer, the hole-blocking layer, or the electron control layer may each independently be from about 20 Å to about 1000 Å, for example, about 30 Å to about 300 Å, and the thickness of the electron transport layer may be from about 100 Å to about 1,000 Å, for example, about 150 Å to about 500 Å. When the thickness of the buffer layer, the hole-blocking layer, the electron control layer, the electron transport layer, and/or the electron transport layer are within these ranges, satisfactory electron transporting characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, an alkaline earth metal complex, or any combination thereof. The metal ion of an alkali metal complex may be a Li ion, a Na ion, a K ion, a Rb ion, or a Cs ion, and the metal ion of an alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

For example, the metal-containing material may include a Li complex. The Li complex may include, for example, Compound ET-D1 (LiQ) or ET-D2:

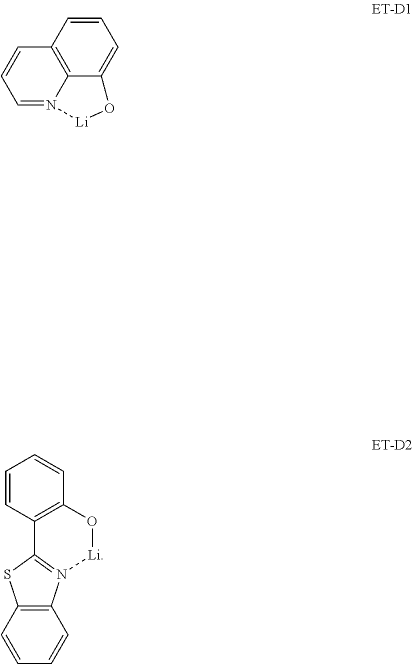

The electron transport region may include an electron injection layer that facilitates the injection of electrons from the second electrode 150. The electron injection layer may directly contact the second electrode 150.

The electron injection layer may have: i) a single-layered structure consisting of a single layer consisting of a single material, ii) a single-layered structure consisting of a single layer consisting of a plurality of different materials, or iii) a multi-layered structure including a plurality of layers including different materials.

The electron injection layer may include an alkali metal, alkaline earth metal, a rare earth metal, an alkali metal-containing compound, alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (for example, fluorides, chlorides, bromides, or iodides), or tellurides of the alkali metal, the alkaline earth metal, and the rare earth metal, or any combination thereof.

The alkali metal-containing compound may include: alkali metal oxides, such as Li₂O, Cs₂O, or K₂O; alkali metal halides, such as LiF, NaF, CsF, KF, LiI, NaI, CsI, or KI; or any combination thereof. The alkaline earth metal-containing compound may include an alkaline earth metal compound, such as BaO, SrO, CaO, BaₓSr₁₋ₓO (wherein x is a real number satisfying the condition of 0<x<1), BaₓCa₁₋ₓO (wherein x is a real number satisfying the condition of 0<x<1), or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal telluride. Examples of the lanthanide metal telluride are LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and Lu₂Te₃.

The alkali metal complex, the alkaline earth-metal complex, and the rare earth metal complex may include i) one of ions of the alkali metal, the alkaline earth metal, and the rare earth metal and ii), as a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

The electron injection layer may consist of an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (for example, a compound represented by Formula 601).

In one or more embodiments, the electron injection layer may consist of: i) an alkali metal-containing compound (for example, an alkali metal halide); or ii) a) an alkali metal-containing compound (for example, an alkali metal halide), and b) an alkali metal, an alkaline earth metal, a rare earth metal, or any combination thereof. For example, the electron injection layer may be a KI:Yb co-deposited layer, an RbI:Yb co-deposited layer, or the like.

When the electron injection layer further includes an organic material, alkali metal, alkaline earth metal, rare earth metal, an alkali metal-containing compound, an alkaline earth metal-containing compound, a rare earth metal-containing compound, alkali metal complex, alkaline earth-metal complex, rare earth metal complex, or any combination thereof may be uniformly or non-uniformly dispersed in a matrix including the organic material.

A thickness of the electron injection layer may be in a range of about 1 Å to about 100 Å, and, for example, about 3 Å to about 90 Å. When the thickness of the electron injection layer is within these ranges, satisfactory electron injection characteristics may be obtained without a substantial increase in driving voltage.

[Second Electrode 150]

The second electrode 150 may be located on the interlayer 130 having a structure as described above. The second electrode 150 may be a cathode, which is an electron injection electrode, and as the material for the second electrode 150, a metal, an alloy, an electrically conductive compound, or any combination thereof, each having a low-work function, may be used.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure including a plurality of layers.

[Capping Layer]

A first capping layer may be located outside the first electrode 110, and/or a second capping layer may be located outside the second electrode 150. In particular, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

Light generated in an emission layer of the interlayer 130 of the light-emitting device 10 may be extracted toward the outside through the first electrode 110 which is a semi-transmissive electrode or a transmissive electrode, and the first capping layer. Light generated in an emission layer of the interlayer 130 of the light-emitting device 10 may be extracted toward the outside through the second electrode 150 which is a semi-transmissive electrode or a transmissive electrode, and the second capping layer.

The first capping layer and the second capping layer may increase external emission efficiency according to the principle of constructive interference. Accordingly, the light extraction efficiency of the light-emitting device 10 is increased, so that the luminescence efficiency of the light-emitting device 10 may be improved.

Each of the first capping layer and the second capping layer may include a material having a refractive index of 1.6 or more (at 589 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer and the second capping layer may each independently include carbocyclic compounds, heterocyclic compounds, amine group-containing compounds, porphyrin derivatives, phthalocyanine derivatives, naphthalocyanine derivatives, alkali metal complexes, alkaline earth metal complexes, or any combination thereof optionally, the carbocyclic compound, the heterocyclic compound, and the amine group-containing compound may be substituted with a substituent including O, N, S, Se, Si, F, Cl, Br, I, or any combination thereof. In one or more embodiments, at least one of the first capping layer and the second capping layer may each independently include an amine group-containing compound.

For example, at least one of the first capping layer and the second capping layer may each independently include a compound represented by Formula 201, a compound represented by Formula 202, or any combination thereof.

In one or more embodiments, at least one of the first capping layer and the second capping layer may each independently include one of Compounds HT28 to HT33, one of Compounds CP1 to CP6, 3-NPB, or any combination thereof:

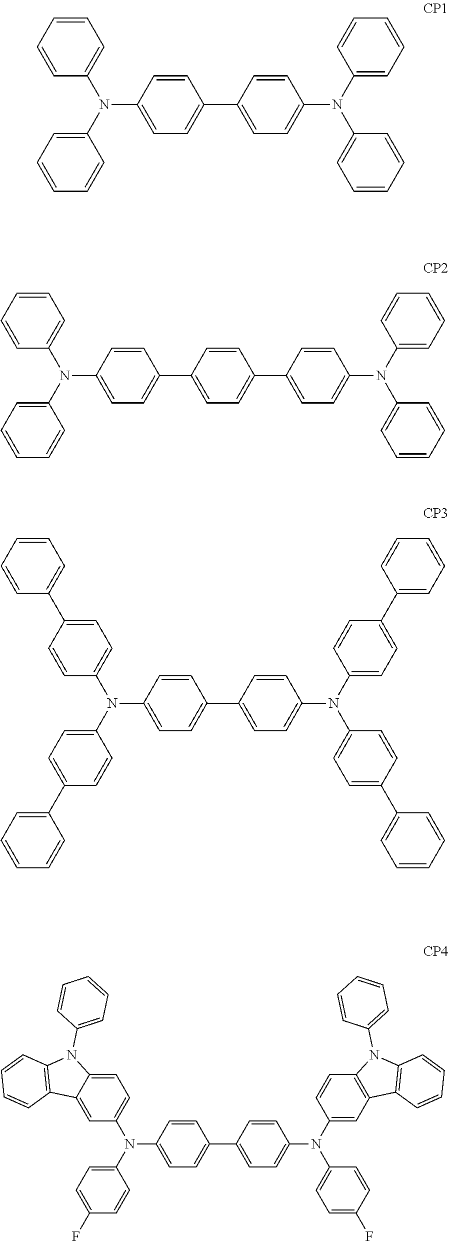 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

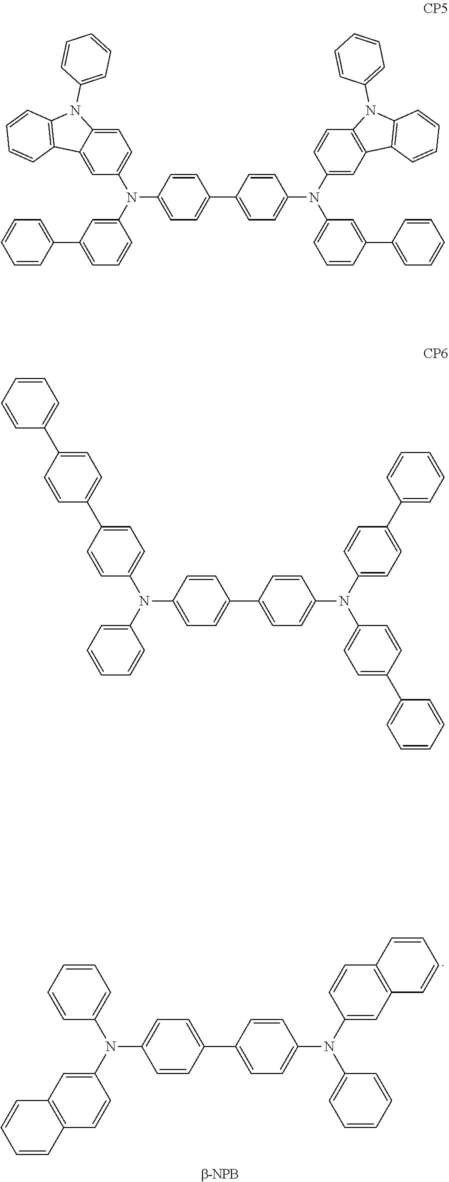 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

[Electronic Apparatus]

The light-emitting device may be included in various electronic apparatuses. For example, the electronic apparatus including the light-emitting device may be a light-emitting apparatus, an authentication apparatus, or the like.

The electronic apparatus (for example, a light-emitting apparatus) may further include, in addition to the light-emitting device, i) a color filter, ii) a color conversion layer, or iii) a color filter and a color conversion layer. The color filter and/or the color conversion layer may be located in at least one traveling direction of light emitted from the light-emitting device. For example, the light emitted from the light-emitting device may be blue light or white light. For details on the light-emitting device, related description provided above may be referred to. In one or more embodiments, the color conversion layer may include a quantum dot. The quantum dot may be, for example, a quantum dot as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be located among the subpixel areas to define each of the subpixel areas.

The color filter may further include a plurality of color filter areas and light-shielding patterns located among the color filter areas, and the color conversion layer may further include a plurality of color conversion areas and light-shielding patterns located among the color conversion areas.

The plurality of color filter areas (or the plurality of color conversion areas) may include a first area emitting first color light, a second area emitting second color light, and/or a third area emitting third color light, wherein the first color light, the second color light, and/or the third color light may have different maximum emission wavelengths from one another. For example, the first color light may be red light, the second color light may be green light, and the third color light may be blue light. For example, the plurality of color filter areas (or the plurality of color conversion areas) may include quantum dots. In particular, the first area may include a red quantum dot, the second area may include a green quantum dot, and the third area may not include a quantum dot. For details on the quantum dot, related descriptions provided herein may be referred to. The first area, the second area, and/or the third area may each include a scatter.

For example, the light-emitting device may emit first light, the first area may absorb the first light to emit first-first color light, the second area may absorb the first light to emit second-first color light, and the third area may absorb the first light to emit third-first color light. In this regard, the first-first color light, the second-first color light, and the third-first color light may have different maximum emission wavelengths. In particular, the first light may be blue light, the first-first color light may be red light, the second-first color light may be green light, and the third-first color light may be blue light.

The electronic apparatus may further include a thin-film transistor, in addition to the light-emitting device as described above. The thin-film transistor may include a source electrode, a drain electrode, and an activation layer, wherein any one of the source electrode and the drain electrode may be electrically connected to any one of the first electrode and the second electrode of the light-emitting device.

The thin-film transistor may further include a gate electrode, a gate insulating film, or the like.

The activation layer may include crystalline silicon, amorphous silicon, an organic semiconductor, an oxide semiconductor, or the like.

The electronic apparatus may further include a sealing portion for sealing the light-emitting device. The sealing portion may be located between the color conversion layer and/or color filter and the light-emitting device. The sealing portion allows light from the light-emitting device to be extracted to the outside, and simultaneously prevents ambient air and moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin film encapsulation layer, the electronic apparatus may be flexible.

Various functional layers may be additionally located on the sealing portion, in addition to the color filter and/or the color conversion layer, according to the use of the electronic apparatus. Examples of the functional layers may include a touch screen layer, a polarizing layer, and the like. The touch screen layer may be a pressure-sensitive touch screen layer, a capacitive touch screen layer, or an infrared touch screen layer. The authentication apparatus may be, for example, a biometric authentication apparatus that authenticates an individual by using biometric information of a living body (for example, fingertips, pupils, etc.).

The authentication apparatus may further include, in addition to the light-emitting device as described above, a biometric information collector.

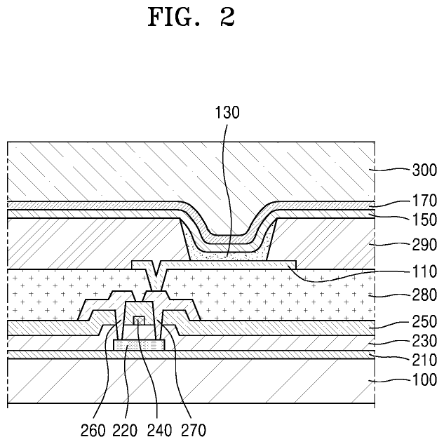

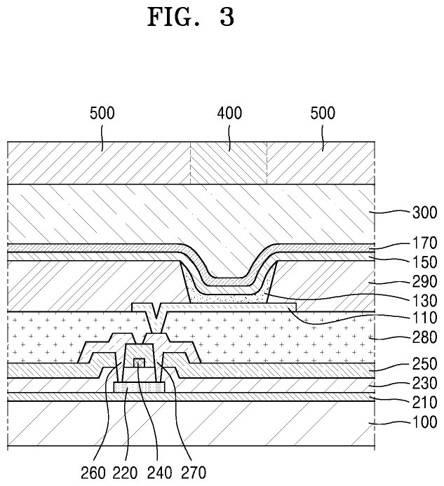

The electronic apparatus may be applied to various displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, various measuring instruments, meters (for example, meters for a vehicle, an aircraft, and a vessel), projectors, and the like.

[Description of FIGS. 2 and 3]

FIG. 2 is a cross-sectional view showing a light-emitting apparatus according to an embodiment.

The light-emitting apparatus of FIG. 2 includes a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be located on the substrate 100. The buffer layer 210 may prevent penetration of impurities through the substrate 100 and may provide a flat surface on the substrate 100.

A TFT may be located on the buffer layer 210. The TFT may include an activation layer 220, a gate electrode 240, a source electrode 260, and a drain electrode 270.

The activation layer 220 may include an inorganic semiconductor such as silicon or polysilicon, an organic semiconductor, or an oxide semiconductor, and may include a source region, a drain region, and a channel region.

A gate insulating film 230 for insulating the activation layer 220 from the gate electrode 240 may be located on the activation layer 220, and the gate electrode 240 may be located on the gate insulating film 230.

An interlayer insulating film 250 may be located on the gate electrode 240. The interlayer insulating film 250 may be located between the gate electrode 240 and the source electrode 260 and between the gate electrode 240 and the drain electrode 270, to insulate from one another.

The source electrode 260 and the drain electrode 270 may be located on the interlayer insulating film 250. The interlayer insulating film 250 and the gate insulating film 230 may be formed to expose the source region and the drain region of the activation layer 220, and the source electrode 260 and the drain electrode 270 may be located in contact with the exposed portions of the source region and the drain region of the activation layer 220.

The TFT is electrically connected to a light-emitting device to drive the light-emitting device, and is covered and protected by a passivation layer 280. The passivation layer 280 may include an inorganic insulating film, an organic insulating film, or any combination thereof. A light-emitting device is provided on the passivation layer 280. The light-emitting device may include a first electrode 110, an interlayer 130, and a second electrode 150.

The first electrode 110 may be located on the passivation layer 280. The passivation layer 280 may be located to expose a portion of the drain electrode 270, not fully covering the drain electrode 270, and the first electrode 110 may be located to be connected to the exposed portion of the drain electrode 270.

A pixel defining layer 290 including an insulating material may be located on the first electrode 110. The pixel defining layer 290 may expose a certain region of the first electrode 110, and an interlayer 130 may be formed in the exposed region of the first electrode 110. The pixel defining layer 290 may be a polyimide or polyacrylic organic film. At least some layers of the interlayer 130 may extend beyond the upper portion of the pixel defining layer 290 to be located in the form of a common layer.

The second electrode 150 may be located on the interlayer 130, and a capping layer 170 may be additionally formed on the second electrode 150. The capping layer 170 may be formed to cover the second electrode 150.

The encapsulation portion 300 may be located on the capping layer 170. The encapsulation portion 300 may be located on a light-emitting device to protect the light-emitting device from moisture or oxygen. The encapsulation portion 300 may include: an inorganic film including silicon nitride (SiNx), silicon oxide (SiOx), indium tin oxide, indium zinc oxide, or any combination thereof, an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, or the like), an epoxy-based resin (for example, aliphatic glycidyl ether (AGE), or the like), or any combination thereof; or any combination of the inorganic films and the organic films.

FIG. 3 shows a cross-sectional view showing a light-emitting apparatus according to an embodiment.

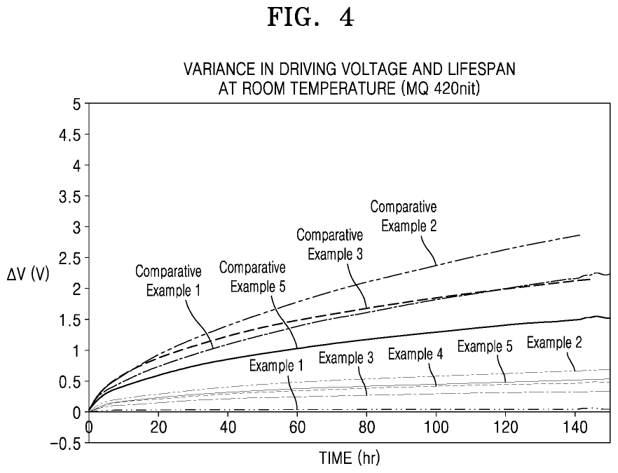

The light-emitting apparatus of FIG. 3 is the same as the light-emitting apparatus of FIG. 2, except that a light-shielding pattern 500 and a functional region 400 are additionally located on the encapsulation portion 300. The functional region 400 may be i) a color filter area, ii) a color conversion area, or iii) a combination of the color filter area and the color conversion area. In an embodiment, the light-emitting device included in the light-emitting apparatus of FIG. 3 may be a tandem light-emitting device.

[Description of FIG. 4]

FIG. 4 is a graph showing the variation in driving voltage (ΔV, V) over time (hr) at luminance of 420 nit with respect to the light-emitting devices of Examples 1 to 5 and Comparative Examples 1 to 5.

Referring to FIG. 4, it is confirmed that the light-emitting devices of Examples 1 to 5 have a lower progressive driving voltage and a longer lifespan than the light-emitting devices of Comparative Examples 1 to 5.

[Manufacturing Method]

The layers included in the hole transport region, the emission layer, and the layers included in the electron transport region may be formed in a certain region by using various methods such as vacuum deposition, spin coating, casting, Langmuir-Blodgett (LB) deposition, ink-jet printing, laser-printing, laser-induced thermal imaging, and the like.

When layers constituting the hole transport region, an emission layer, and layers constituting the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### Definition of Terms

The term “C₃-C₆₀ carbocyclic group” as used herein refers to a cyclic group consisting of carbon only as a ring-forming atom and having three to sixty carbon atoms, and the term “C₁-C₆₀ heterocyclic group” as used herein refers to a cyclic group that has one to sixty carbon atoms and further has, in addition to carbon, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed with each other. For example, the C₁-C₆₀ heterocyclic group has 3 to 61 ring-forming atoms.

The “cyclic group” as used herein may include the C₃-C₆₀ carbocyclic group, and the C₁-C₆₀ heterocyclic group.

The term “π0 electron-rich C₃-C₆₀ cyclic group” as used herein refers to a cyclic group that has three to sixty carbon atoms and does not include *—N=*′ as a ring-forming moiety, and the term “π electron-deficient nitrogen-containing C₁-C₆₀ cyclic group” as used herein refers to a heterocyclic group that has one to sixty carbon atoms and includes *—N=*′ as a ring-forming moiety.

For example,
the C₃-C₆₀ carbocyclic group may be i) a T1 group or ii) a condensed cyclic group in which two or more T1 groups are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) a T2 group, ii) a condensed cyclic group in which two or more T2 groups are condensed with each other, or iii) a condensed cyclic group in which at least one T2 group and at least one T1 group are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and the like),
the π electron-rich C₃-C₆₀ cyclic group may be i) a T1 group, ii) a condensed cyclic group in which two or more T2 groups are condensed with each other, iii) a T3 group, iv) a condensed cyclic group in which two or more T3 groups are condensed with each other, or v) a condensed cyclic group in which at least one T3 group and at least one T1 group are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, and the like), the π electron-deficient nitrogen-containing C₁-C₆₀ cyclic group may be i) a T4 group, ii) a condensed cyclic group in which two or more T4 groups are condensed with each other, iii) a condensed cyclic group in which at least one T4 group and at least one T1 group are condensed with each other, iv) a condensed cyclic group in which at least one T4 group and at least one T3 group are condensed with each other, or v) a condensed cyclic group in which at least one T4 group, at least one T1 group, and at least one T3 group are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and the like),
the T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group, the T2 group may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group, the T3 group may be a furan group, a thiophene group, a 1H-pyrrole group, a silole group, or a borole group, and
the T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group, the C₃-C₆₀ carbocyclic group, the C₁-C₆₀ heterocyclic group, the π electron-rich C₃-C₆₀ cyclic group, or the π electron-deficient nitrogen-containing C₁-C₆₀ cyclic group” as used herein refer to a group condensed to any cyclic group, a monovalent group, or a polyvalent group (for example, a divalent group, a trivalent group, a tetravalent group, and the like) according to the structure of a formula for which the corresponding term is used. For example, the “benzene group” may be a benzo group, a phenyl group, a phenylene group, or the like, which may be easily understood by one of ordinary skill in the art according to the structure of a formula including the “benzene group”.

Examples of the monovalent C₃-C₆₀ carbocyclic group and the monovalent C₁-C₆₀ heterocyclic group are a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Examples of the divalent C₃-C₆₀ carbocyclic group and the monovalent C₁-C₆₀ heterocyclic group are a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group” as used herein refers to a linear or branched aliphatic hydrocarbon monovalent group that has one to sixty carbon atoms, and specific examples thereof are a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as used herein refers to a divalent group having the same structure as the C₁-C₆₀ alkyl group.

The term “C₂-C₆₀ alkenyl group” as used herein refers to a monovalent hydrocarbon group having at least one carbon-carbon double bond in the middle or at the terminus of the C₂-C₆₀ alkyl group, and examples thereof are an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a monovalent hydrocarbon group having at least one carbon-carbon triple bond in the middle or at the terminus of the C₂-C₆₀ alkyl group, and examples thereof include an ethynyl group and a propynyl group. The term “C₂-C₆₀ alkynylene group” as used herein refers to a divalent group having the same structure as the C₂-C₆₀ alkynyl group.

The term “C₁-C₆₀ alkoxy group” as used herein refers to a monovalent group represented by —OA₁₀₁ (wherein A₁₀₁ is the C₁-C₆₀ alkyl group), and examples thereof include a methoxy group, an ethoxy group, and an isopropyloxy group.

The term “C₃-C₁₀ cycloalkyl group” as used herein refers to a monovalent saturated hydrocarbon cyclic group having 3 to 10 carbon atoms, and examples thereof are a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, and a bicyclo[2.2.2]octyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent cyclic group of 1 to 10 carbon atoms, further including, in addition to carbon atoms, at least one heteroatom, as ring-forming atoms, and specific examples are a 1,2,3,4-oxatriazolidinyl group, a tetrahydrofuranyl group, and a tetrahydrothiophenyl group. The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent cyclic group that has three to ten carbon atoms and at least one carbon-carbon double bond in the ring thereof and no aromaticity, and specific examples thereof are a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent cyclic group of 1 to 10 carbon atoms, further including, in addition to carbon atoms, at least one heteroatom, as ring-forming atoms, and having at least one carbon-carbon double bond in the cyclic structure thereof. Examples of the C₁-C₁₀ heterocycloalkenyl group are a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system of 6 to 60 carbon atoms, and the term “C₆-C₆₀ arylene group” as used herein refers to a divalent group having a carbocyclic aromatic system of 6 to 60 carbon atoms. Examples of the C₆-C₆₀ aryl group are a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each include two or more rings, the rings may be condensed with each other.

The term “C₁-C₆₀ heteroaryl group” as used herein refers to a monovalent group having a heterocyclic aromatic system of 1 to 60 carbon atoms, further including, in addition to carbon atoms, at least one heteroatom, as ring-forming atoms. The term “C₁-C₆₀ heteroarylene group” as used herein refers to a divalent group having a heterocyclic aromatic system of 1 to 60 carbon atoms, further including, in addition to carbon atoms, at least one heteroatom, as ring-forming atoms. Examples of the C₁-C₆₀ heteroaryl group are a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the rings may be condensed with each other.

The term “monovalent non-aromatic condensed polycyclic group” as used herein refers to a monovalent group (for example, having 8 to 60 carbon atoms) having two or more rings condensed to each other, only carbon atoms as ring-forming atoms, and no aromaticity in its entire molecular structure. Examples of the monovalent non-aromatic condensed polycyclic group are an indenyl group, a fluorenyl group, a spiro-bifluorenyl group, a benzofluorenyl group, an indenophenanthrenyl group, and an indeno anthracenyl group. The term “divalent non-aromatic condensed polycyclic group” as used herein refers to a divalent group having the same structure as the monovalent non-aromatic condensed polycyclic group described above.

The term “monovalent non-aromatic condensed heteropolycyclic group” as used herein refers to a monovalent group (for example, having 1 to 60 carbon atoms) having two or more rings condensed to each other, further including, in addition to carbon atoms, at least one heteroatom, as ring-forming atoms, and having non-aromaticity in its entire molecular structure. Examples of the monovalent non-aromatic condensed heteropolycyclic group are a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group. The term “divalent non-aromatic condensed heteropolycyclic group” as used herein refers to a divalent group having the same structure as the monovalent non-aromatic condensed heteropolycyclic group described above.

The term “C₆-C₆₀ aryloxy group” as used herein indicates —OA₁₀₂ (wherein A₁₀₂ is the C₆-C₆₀ aryl group), and the term “C₆-C₆₀ arylthio group” as used herein indicates —SA₁₀₃ (wherein A₁₀₃ is the C₆-C₆₀ aryl group).

The term “C₇-C₆₀ aryl alkyl group” used herein refers to -A₁₀₄A₁₀₅ (where A₁₀₄ may be a C₁-C₅₄ alkylene group, and A₁₀₅ may be a C₆-C₅₉ aryl group), and the term C₂-C₆₀ heteroaryl alkyl group” used herein refers to -A₁₀₆A₁₀₇ (where A₁₀₆ may be a C₁-C₅₉ alkylene group, and A₁₀₇ may be a C₁-C₅₉ heteroaryl group).

The term “R₁₀ₐ” as used herein may be:
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof,
a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

In the one or more of the embodiments described herein, Q₁ to Q₃, Q₁₁ to Q₁₃, Q₂₁ to Q₂₃ and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ aryl alkyl group; or a C₂-C₆₀ heteroaryl alkyl group.

The term “heteroatom” as used herein refers to any atom other than a carbon atom and a hydrogen atom. Examples of the heteroatom are O, S, N, P, Si, B, Ge, Se, or any combinations thereof.

The term “third-row transition metal” used herein includes hafnium (Hf), tantalum (Ta), tungsten (W), rhenium (Re), osmium (Os), iridium (Ir), platinum (Pt), gold (Au), and the like.

“Ph” as used herein refers to a phenyl group, “Me” as used herein refers to a methyl group, “Et” as used herein refers to an ethyl group, “ter-Bu” or “Bu^{t}” as used herein refers to a tert-butyl group, and “OMe” as used herein refers to a methoxy group.

The term “biphenyl group” as used herein refers to “a phenyl group substituted with a phenyl group”. In other words, the “biphenyl group” is a substituted phenyl group having a C₆-C₆₀ aryl group as a substituent.

The term “terphenyl group” as used herein refers to “a phenyl group substituted with a biphenyl group”. In other words, the “terphenyl group” is a substituted phenyl group having, as a substituent, a C₆-C₆₀ aryl group substituted with a C₆-C₆₀ aryl group.

* and *′ as used herein, unless defined otherwise, each refer to a binding site to a neighboring atom in a corresponding formula or moiety.

Hereinafter, compounds according to embodiments and light-emitting devices according to embodiments will be described in detail with reference to the following synthesis examples and examples. The wording “B was used instead of A” used in describing Synthesis Examples denote that an identical molar equivalent of B was used in place of A.

### EXAMPLES

### Synthesis Example A: Synthesis of Intermediate C(1)

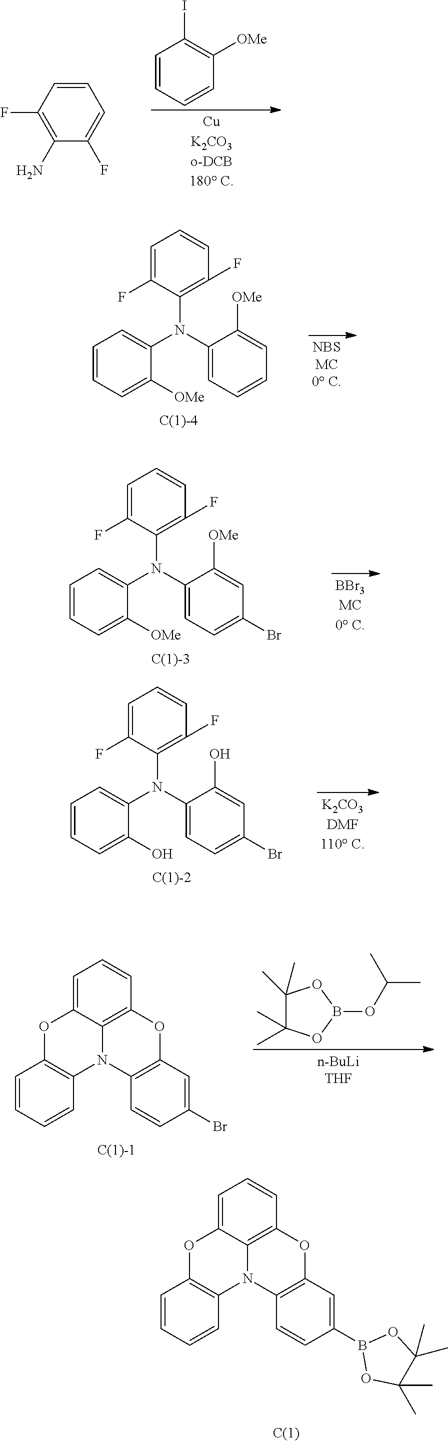

### Synthesis of Intermediate C(1)-4

2,6-difluoroaniline (4.55 g, 1 eq), 1-iodo-2-methoxybenzene (17 g, 2.1 eq), Cu (4.6 g, 2 eq), K₂CO₃ (12.5 g, 2.6 eq), and 60 ml of o-DCB were added to a 1-neck round-bottom flask, and stirred at 180° C. for 3 days. A residue obtained after completion of the reaction was separated and purified by column chromatography using, as a developing solvent, a mixture of methylene chloride and hexane (at a volume ratio of 1:2), so as to obtain about 6.7 g of Intermediate C(1)-4 (yield: 56%).

### Synthesis of Intermediate C(1)-3

Intermediate C(1)-4 (6.7 g, 1 eq), NBS (N-Bromosuccinimide) (4.9 g, 1 eq), and 150 ml of methylene chloride (MC) were added to a 1-neck round-bottom flask, and stirred at room temperature for 12 hours. A residue obtained after completion of the reaction was separated and purified by column chromatography using, as a developing solvent, a mixture of MC and hexane (at a volume ratio of 1:5), so as to obtain about 7 g of Intermediate C(1)-3 (yield: 85%).

### Synthesis of Intermediate C(1)-2

Intermediate C(1)-3 (7 g, 1 eq) and 250 ml of MC were added to a 3-neck round-bottom flask. While stirring at −78° C., BBr₃ (3.89 ml, 2.5 eq) dissolved in 60 ml of MC was added dropwise thereto. After completion of the addition by dropwise, the resulting solution was stirred at 0° C. for 4 hours. After completion of the reaction, an extraction process using MC was performed thereon. An organic layer obtained therefrom was dried using MgSO₄, and the solvent was evaporated, so as to obtain about 6 g (yield: 92%) of Intermediate C(1)-2.

### Synthesis of Intermediate C(1)-1

Intermediate C(1)-2 (6 g, 1 eq), K₂CO₃ (6.34 g, 3 eq), and 200 ml of DMF (dimethylformamide) were added to a 1-neck round-bottom flask, and stirred at 110° C. for 8 hours. After completion of the reaction, DMF was removed therefrom. Subsequently, through silica gel short-path column, a solid obtained by removing the solvent by distillation under reduced pressure was recrystallized with ether, so as to obtain about 4.8 g (yield: 89%) of Intermediate C(1)-1.

### Synthesis of Intermediate C(1)

Intermediate C(1)-1 (2 g, 1 eq) and 20 ml of THE (tetrahydrofuran) were added to a 3-neck round-bottom flask, and n-BuLi (2.7 ml, 1.2 eq) was added thereto at −78° C. Then, the resulting solution was stirred for 30 minutes. Subsequently, 2-isopropoxy-4,4,5,5-tetramethyl-1,3,2-dioxaborolane (1.7 ml, 1.5 eq) was added dropwise thereto. After completion of the addition by dropwise, the resulting solution was stirred at room temperature for 3 hours. A residue obtained after completion of the reaction was separated and purified by column chromatography using, as a developing solvent, a mixture of ethyl acetate (EA) and hexane (at a volume ratio of 1:2), so as to obtain about 0.4 g (yield: 20%) of Intermediate C(1).

Synthesis Example B: Synthesis of Intermediate C(2)

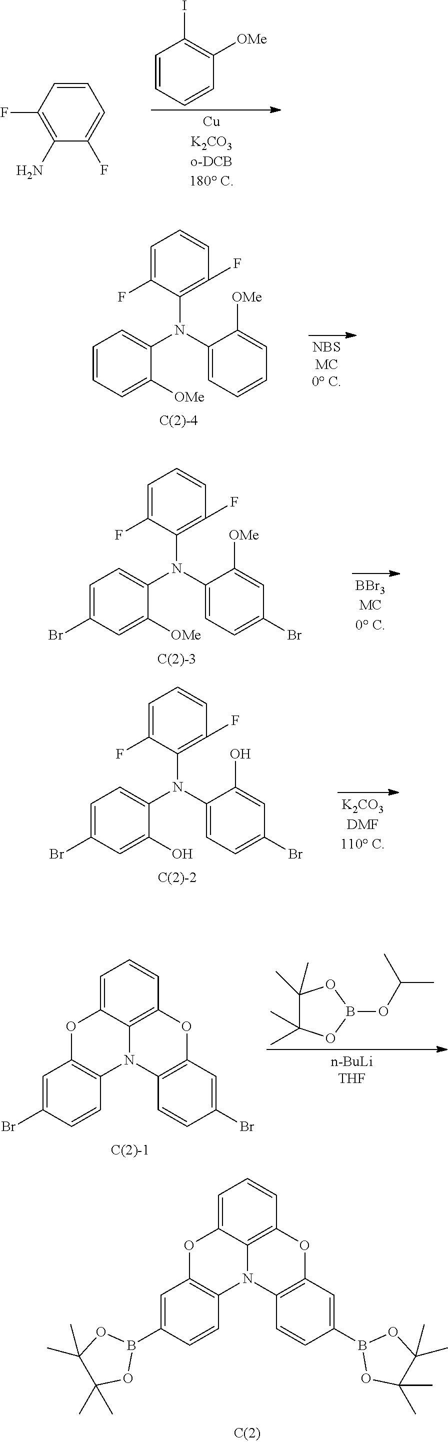

Intermediate C(2) was synthesized in the same manner as in Synthesis Example A, except that, in the synthesis of Intermediate C(1), 2 eq of NBS was used instead of 1 eq thereof in synthesizing Intermediate C(1)-3 and that 3 eq of 2-isopropoxy-4,4,5,5-tetramethyl-1,3,2-dioxaborolane was used instead of 1.5 eq thereof in synthesizing Intermediate C(2).

### Synthesis Example C: Synthesis of Intermediate C(3)

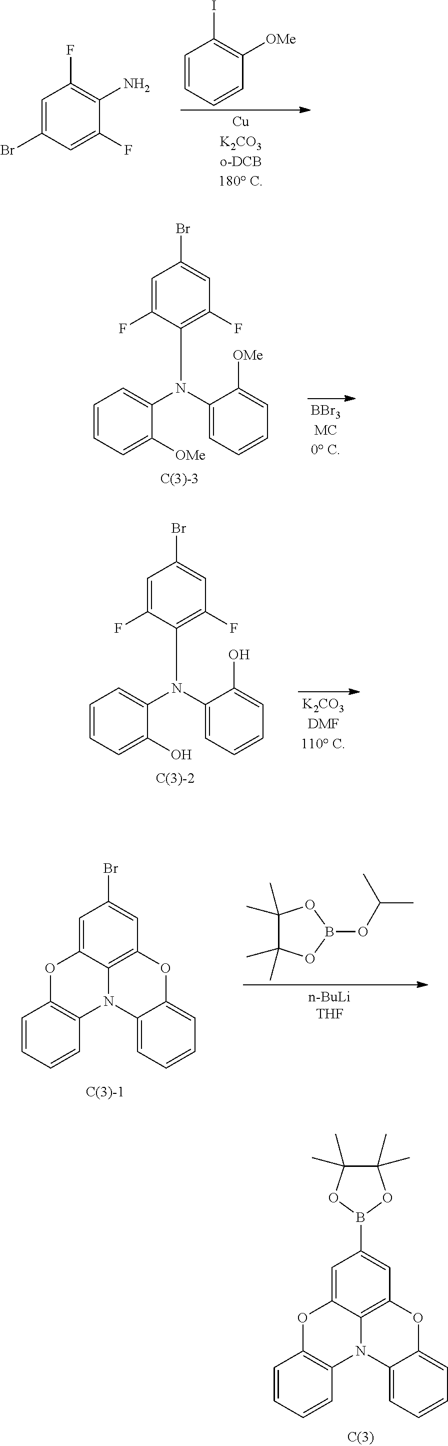

Intermediate C(3) was synthesized in the same manner as in Synthesis Example A in synthesizing Intermediate C(1)-4, except that, in the synthesis of Intermediate C(1), 4-bromo-2,6-difluoroaniline was used instead of 2,6-difluoroaniline and that the synthesis of Intermediate C(1)-3 was excluded.

### Synthesis Example D: Synthesis of Intermediate C(4)

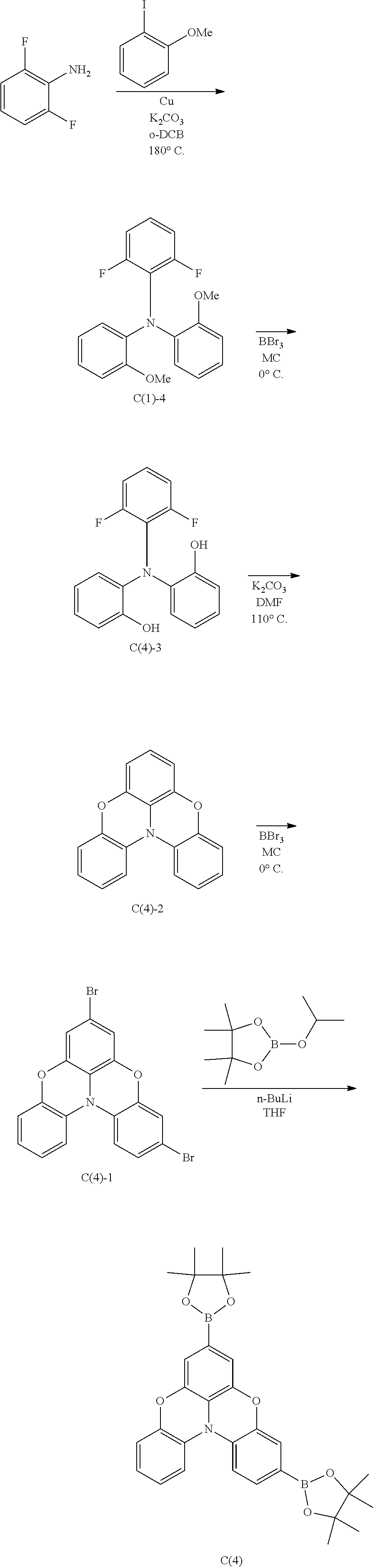

Intermediate C(4)-2 was synthesized in the same manner as in the synthesis of Intermediate C(1), except that the synthesis of Intermediate C(1)-3 was excluded. In addition, Intermediate C(4) was synthesized in the same manner as in Synthesis Example A, except that 2 eq of NBS was used instead of 1 eq thereof as of Intermediate C(1)-3 to synthesize Intermediate C(4)-1 and that 3 eq of 2-isopropoxy-4,4,5,5,-tetramethyl-1,3,2-dioxaborolane was used instead of 1.5 eq thereof as used in the synthesis of Intermediate C(1) to synthesize Intermediate C(4).

### Synthesis Example 1: Synthesis of Compound 1

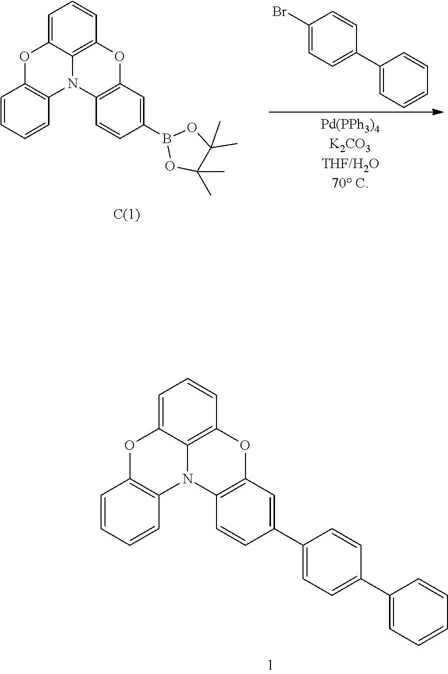 [SMILES: CC1(C)OB(c2ccc3c(c2)Oc2cccc4c2N3c2ccccc2O4)OC1(C)C ; Brc1ccc(-c2ccccc2)cc1 ; c1ccc(-c2ccc(-c3ccc4c(c3)Oc3cccc5c3N4c3ccccc3O5)cc2)cc1]

Intermediate C(1) (1.8 g, 1 eq), 4-bromo-1,1′-biphenyl (1.3 g, 1.2 eq), Pd(PPh₃)₄ (0.5 g, 0.05 eq), K₂CO₃ (2.4 g, 3 eq), and THF/H₂O (40 ml/10 ml) were added to a 1-neck round-bottom flask, and stirred at 70° C. for 12 hours. The mixture after completion of the reaction was separated and purified by column chromatography using, as a developing solvent, a mixture of MC and hexane (at a volume ratio of 1:5), so as to obtain about 1.1 g of Compound 1 (yield: 58%, purity: 99%). Afterwards, a sublimation purification process was performed thereon to obtain about 1 g of Compound 1 (purity: 99.99%).

### Synthesis Example 2: Synthesis of Compound 5

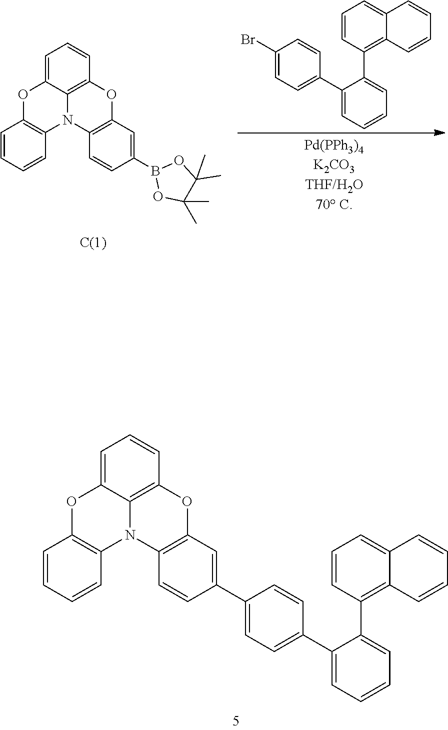 [SMILES: CC1(C)OB(c2ccc3c(c2)Oc2cccc4c2N3c2ccccc2O4)OC1(C)C ; Brc1ccc(-c2ccccc2-c2cccc3ccccc23)cc1 ; c1ccc2c(c1)Oc1cccc3c1N2c1ccc(-c2ccc(-c4ccccc4-c4cccc5ccccc45)cc2)cc1O3]

Compound 5 was synthesized in the same manner as in Synthesis Example 1, except that 2.4 eq of a halogen compound shown in the scheme above was used instead of 4-bromo-1,1′-biphenyl.

### Synthesis Example 3: Synthesis of Compound 30

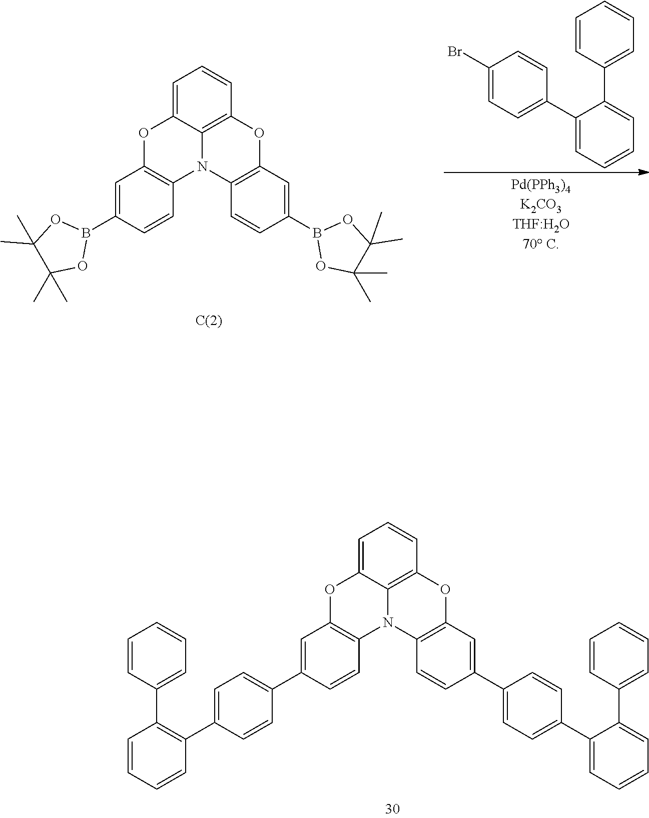

Compound 30 was synthesized in the same manner as in Synthesis Example 1, except that Intermediate C(2) was used instead of Intermediate C(1) and that 2.4 eq of a halogen compound shown in the scheme above was used instead of 4-bromo-1,1′-biphenyl.

### Synthesis Example 4: Synthesis of Compound 62

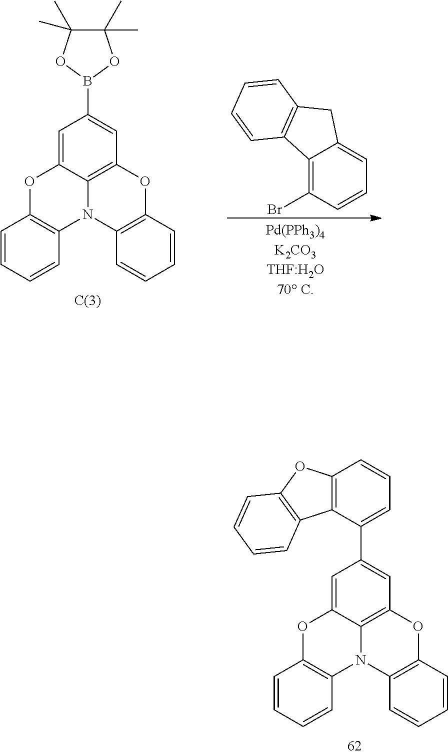 [SMILES: CC1(C)OB(c2cc3c4c(c2)Oc2ccccc2N4c2ccccc2O3)OC1(C)C ; Brc1cccc2c1-c1ccccc1C2 ; c1ccc2c(c1)Oc1cc(-c3cccc4oc5ccccc5c34)cc3c1N2c1ccccc1O3]

Compound 62 was synthesized in the same manner as in Synthesis Example 1, except that Intermediate C(3) was used instead of Intermediate C(1) and that a halogen compound shown in the scheme above was used instead of 4-bromo-1,1′-biphenyl.

### Synthesis Example 5: Synthesis of Compound 90

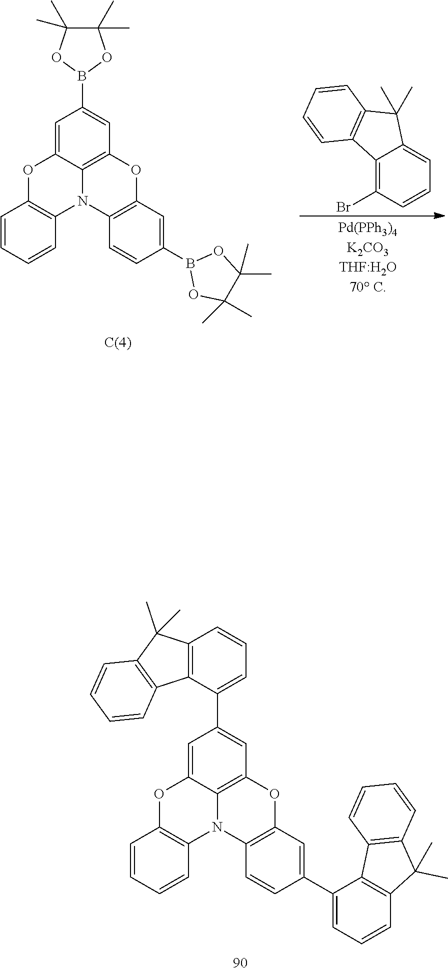 [SMILES: CC1(C)OB(c2ccc3c(c2)Oc2cc(B4OC(C)(C)C(C)(C)O4)cc4c2N3c2ccccc2O4)OC1(C)C ; CC1(C)c2ccccc2-c2c(Br)cccc21 ; CC1(C)c2ccccc2-c2c(-c3ccc4c(c3)Oc3cc(-c5cccc6c5-c5ccccc5C6(C)C)cc5c3N4c3ccccc3O5)cccc21]

Compound 90 was synthesized in the same manner as in Synthesis Example 1, except that Intermediate C(4) was used instead of Intermediate C(1) and that 2.4 eq of a halogen compound shown in the scheme above was used instead of 4-bromo-1,1′-biphenyl.

¹H NMR and MS/FAB of the compounds synthesized according to Synthesis Examples 1 to 5 are shown in Table 1. Synthesis methods for other compounds than the compounds shown in Table 1 may be easily recognized by those skilled in the technical field by referring to the synthesis paths and source material materials described above.

TABLE 1

| | | MS/FAB | |
|---|---|---|---|
| Compound | ¹H NMR (δ) | Calc | Found |
| 1 | 7.75 (dd, 2H), 7.49-7.25 (m, 10H), 7.14 (d, 1H), 7.01-6.96 | 425.49 | 425.14 |
| | (m, 4H), 6.68 (d, 2H) | | |
| 5 | 8.95(d, 1H), 8.50(d,1H), 8.20(d, 1H), 8.09(d, 1H), 7.96(d, | 551.19 | 552.11 |
| | 2H), 7.77(t, 1H), 7.60-7.52(m, 3H), 7.39-7.25(m,8H), | | |
| | 7.14(d, 1H),7.01-6.96(m, 4H), 6.68(d, 2H) | | |
| 30 | 7.96(d, 4H), 7.79(d, 4H), 7.60(d, 4H), 7.46-7.25(m, 20H), | 729.27 | 730.21 |
| | 6.97(t, 1H), 6.68(d, 2H) | | |
| 62 | 7.98(d, 1H), 7.82(d, 1H), 7.69(d, 1H), 7.57-7.54(m, 2H), | 439.47 | 440.50 |
| | 7.39-7.31(m, 2H), 7.14(d, 2H), 7.01-6.95(m, 8H) | | |
| 90 | 7.90(d, 2H), 7.78(d, 2H), 7.65(d, 2H), 7.55(d, 2H), 7.47- | 657.27 | 658.33 |
| | 7.27(m, 9H), 7.14(d, 1H), 7.01-6.96(m, 5H), 1.69(s, 12H) | | |

### Example 1

As an anode, ITO was deposited on a glass substrate to a thickness of 100 Å by sputtering and Ag and WOₓ (where x is between 2.5 and 3.0) were deposited thereon to a thickness of 800 Å and 100 Å, respectively, by sputtering, thereby completing the formation of an anode. Then, the anode was subjected to plasma treatment using nitrogen gas and oxygen gas at a volume ratio of 1:1 under conditions of power of 200 W and pressure of 5 m torr for 60 seconds, and a PT process was performed thereon.

Compound 1 was deposited on the anode to form a hole transport layer having a thickness of 1,100 Å.

9,10-Di(2-naphthyl)anthracene (ADN) as a host compound and 4,4′-bis[2-(4-(N,N-diphenylamino)phenyl)vinyl]biphenyl (DPAVBi) as a dopant compound were co-deposited at a weight ratio of 97:3 on the hole transport layer to form an emission layer having a thickness of 300 Å.

2,4,6-Tris(3-(pyrimidin-5-yl)phenyl)-1,3,5-triazine (TPM-TAZ) and LiQ were co-deposited at a weight ratio of 5:5 on the emission layer to form an electron transport layer having a thickness of 100 Å.

Yb was vacuum-deposited on the electron transport layer to a thickness of 10 Å, and subsequently, Ag—Mg was vacuum-deposited thereon to form a cathode having a thickness of 100 Å, and CP1 was deposited on the cathode to form a capping layer having a thickness of 700 Å, thereby completing the manufacture of a light-emitting device.

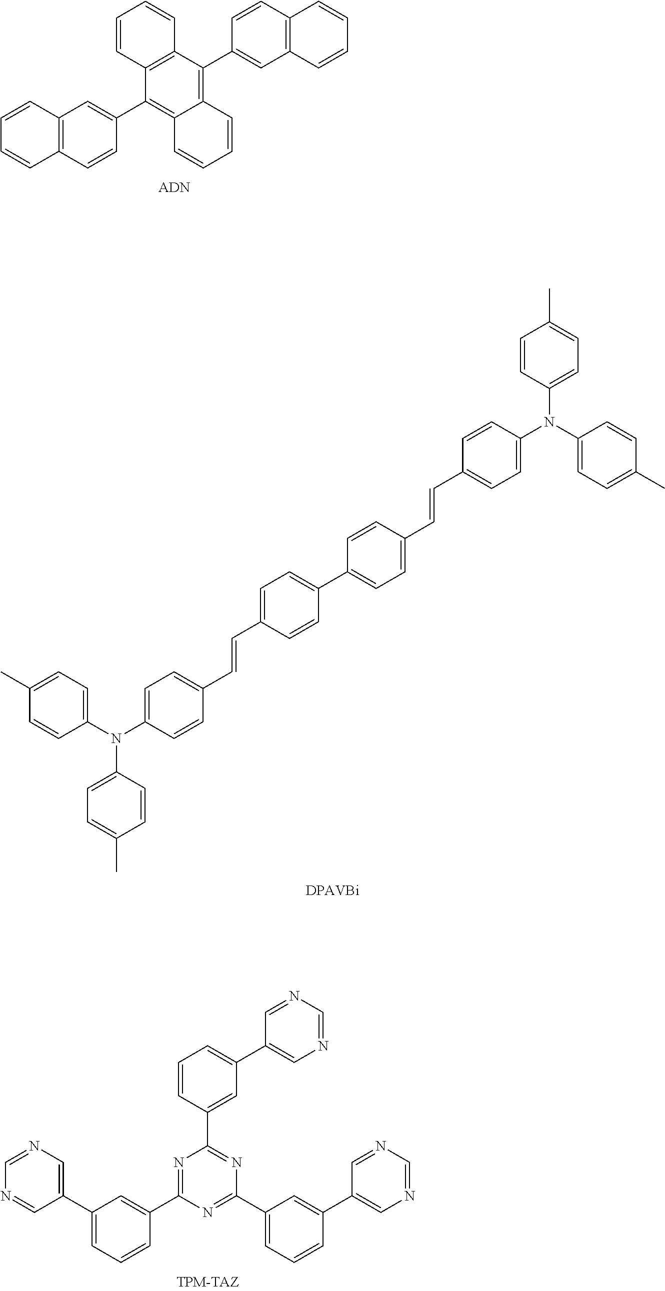

### Examples 2 to 5

Light-emitting devices were manufactured in the same manner as in Example 1, except that compounds of Table 2 were respectively used instead of Compound 1 in forming a hole transport layer.

### Example 6

A light-emitting device was manufactured in the same manner as in Example 1, except that MoOₓ (where x is between 2.5 and 3.0) instead of WOₓ (where x is between 2.5 to 3.0) was deposited to a thickness of 100 Å by sputtering in forming an anode.

### Comparative Example 1

A light-emitting device was manufactured in the same manner as in Example 1, except that an ITO glass substrate (Corning, 15 Ω/cm² (1,200 Å)) was used as an anode.

### Comparative Example 2

A light-emitting devices was manufactured in the same manner as in Comparative Example 1, except that Compound 5 was used instead of Compound 1 in forming a hole transport layer.

### Comparative Example 3

A light-emitting devices was manufactured in the same manner as in Comparative Example 1, except that Compound 30 was used instead of Compound 1 in forming a hole transport layer.

### Comparative Example 4

A light-emitting devices was manufactured in the same manner as in Comparative Example 1, except that Compound HT1 was used instead of Compound 1 in forming a hole transport layer.

### Comparative Examples 5 to 7

Light-emitting devices were manufactured in the same manner as in Example 1, except that compounds of Table 2 were respectively used instead of Compound 1 in forming a hole transport layer.

### Evaluation Example 1

Table 2 shows the work function of the anode and the measured HOMO energy level value (eV) of the hole transport layer of each of the light-emitting devices manufactured in Examples 1 to 6 and Comparative Examples 1 to 7.

TABLE 2

| | | Work | | HOMO energy |
|---|---|---|---|---|
| | | function of | | level of hole |
| | | anode | Hole transport | transport layer |
| | Anode | (eV) | layer | (eV) |
| Example 1 | ITO/Ag/WO_{X} | 5.35 | Compound 1 | 5.25 |
| Example 2 | | | Compound 5 | 5.34 |
| Example 3 | | | Compound 30 | 5.26 |
| Example 4 | | | Compound 62 | 5.27 |
| Example 5 | | | Compound 90 | 5.30 |
| Example 6 | ITO/Ag/MoO_{X} | 5.33 | Compound 1 | 5.25 |
| Comparative | ITO | 4.8 | Compound 1 | 5.25 |
| Example 1 | | | | |
| Comparative | | | Compound 5 | 5.34 |
| Example 2 | | | | |
| Comparative | | | Compound 30 | 5.26 |
| Example 3 | | | | |
| Comparative | | | HT1 | 5.15 |
| Example 4 | | | | |
| Comparative | ITO/Ag/WO_{X} | 5.35 | HT1 | 5.15 |
| Example 5 | | | | |
| Comparative | | | HT2 | 5.17 |
| Example 6 | | | | |
| Comparative | | | HT3 | 5.21 |
| Example 7 | | | | |

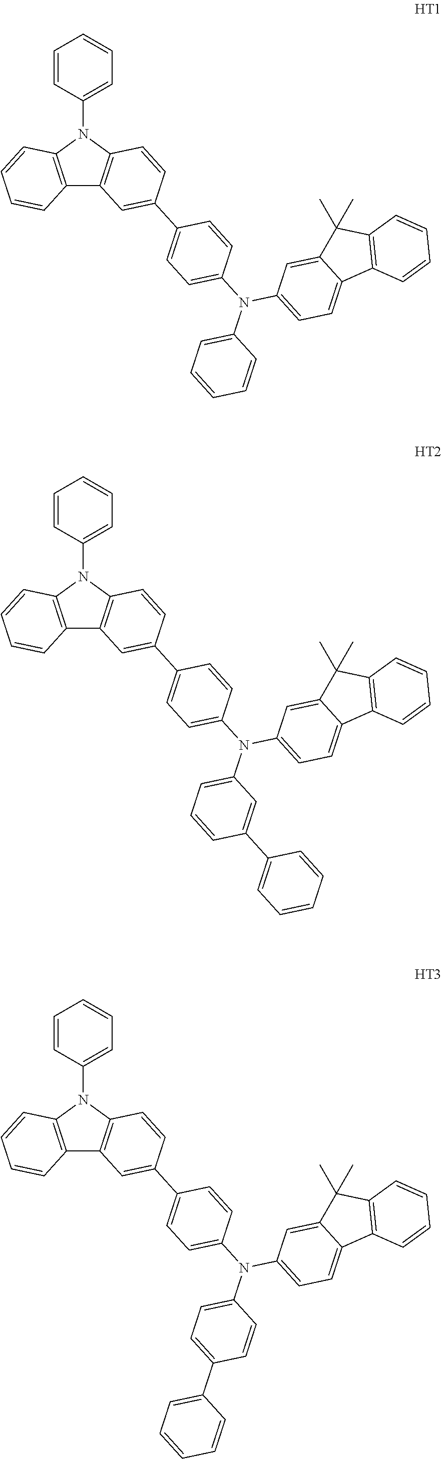 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

### Evaluation Example 2

Using a source meter (Keithley Instrument Inc., 2400 series) at current density of 10 mA/cm² of the light-emitting devices of Examples 1 to 5 and Comparative Examples 1 to 5, one point every 10 minutes was measured under conditions of room temperature and luminance of MQ 420 nits. The variation in the driving voltage over time (hr) for a total of 150 hours was measured by measuring the points, and results thereof are shown in FIG. 4.

### Evaluation Example 3

Regarding the light-emitting devices of Examples 1 to 6 and Comparative Examples 1 to 7, the driving voltage at current density of 10 mA/cm², luminescence efficiency, and lifespan (T₉₅) were measured. The driving voltage of each of the light-emitting devices was measured using a source meter (Keithley Instrument Inc., 2400 series), and the luminescence efficiency thereof was measured using a luminescence efficiency measurement apparatus C9920-2-12 of Hamamatsu Photonics Inc. For the luminescence efficiency evaluation, a luminance meter after wavelength-sensitivity calibration was used to measure the luminance/current density. The lifespan of each of the light-emitting devices was measured based on the time when the maximum luminance was reduced to 95%.

In addition, the progressive driving voltage (ΔV) of each of the light-emitting devices was measured using a source meter (Keithley Instrument Inc., 2400 series) at current density of 10 mA/cm² of the light-emitting device while applying the same current thereto under conditions of room temperature and luminance of MQ 420 nits. Then, the variation in the driving voltage after a total of 150 hours was measured. Table 3 shows the evaluation results of the characteristics of the light-emitting devices.

TABLE 3

| | | | Driving | | | |
|---|---|---|---|---|---|---|
| | | Hole | voltage | Luminescence | | |
| | | transport | (V, @10 | efficiency | | ΔV(V, |
| | Anode | layer | mA/cm²) | (d/A) | LT(T₉₅) | @150 hr) |
| Example 1 | ITO/Ag/WOₓ | Compound1 | 3.9 | 6.1 | 80 | 0.2 |
| Example 2 | ITO/Ag/WOₓ | Compound 5 | 4.2 | 5.8 | 84 | 0.6 |
| Example 3 | ITO/Ag/WOₓ | Compound 30 | 4.3 | 6.6 | 90 | 0.1 |
| Example 4 | ITO/Ag/WOₓ | Compound 62 | 4.1 | 6.7 | 68 | 0.5 |
| Example 5 | ITO/Ag/WOₓ | Compound 90 | 4.4 | 6.5 | 75 | 0.5 |
| Example 6 | ITO/Ag/MoOₓ | Compound 1 | 4.2 | 5.7 | 60 | 0.3 |
| Comparative | ITO | Compound 1 | 4.7 | 6.4 | 21 | 2.3 |
| Example 1 | | | | | | |
| Comparative | ITO | Compound 5 | 4.9 | 6.9 | 18 | 2.9 |
| Example 2 | | | | | | |
| Comparative | ITO | Compound 30 | 5.3 | 6.6 | 27 | 2.2 |
| Example 3 | | | | | | |
| Comparative | ITO | HT1 | 4.3 | 6.3 | 30 | 2 |
| Example 4 | | | | | | |
| Comparative | ITO/Ag/WOₓ | HT1 | 4.4 | 6.4 | 54 | 1.5 |
| Example 5 | | | | | | |
| Comparative | ITO/Ag/WOₓ | HT2 | 4.3 | 6.5 | 41 | 1.5 |
| Example 6 | | | | | | |
| Comparative | ITO/Ag/WOₓ | HT3 | 4.4 | 6.7 | 30 | 1.7 |
| Example 7 | | | | | | |

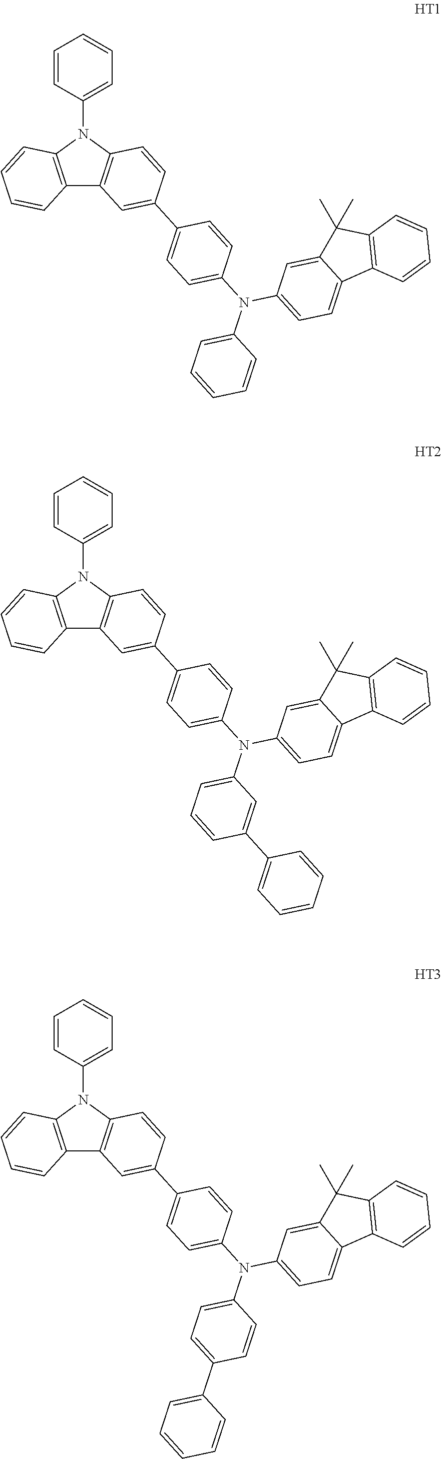 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

Referring to Table 3, it was confirmed that the light-emitting devices of Examples 1 to 6 had the same or lower driving voltage, higher luminescence efficiency, and longer lifespan than the light-emitting devices of Comparative Examples 1 to 7, and also had a reduced progressive driving voltage.

As described above, according to the one or more embodiments, a light-emitting device may include a first electrode including an inorganic material and a hole transport layer including a condensed cyclic compound represented by Formula 1, and accordingly, the light-emitting device may have improved device stability, low progressive driving voltage, high luminescence efficiency, and long lifespan characteristics.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. A light-emitting device comprising:
a first electrode;
a second electrode facing the first electrode; and
an interlayer between the first electrode and the second electrode and comprising an emission layer,
wherein the first electrode comprises an inorganic material,
the inorganic material comprises a metal oxide that includes at least one metal selected from W, Mo, Ga, Ni, Cu, Zn, and Ti,
the interlayer comprises a hole transport region between the first electrode and the emission layer,
the hole transport region comprises a hole transport layer, and
the hole transport layer comprises at least one condensed cyclic compound represented by Formula 1: 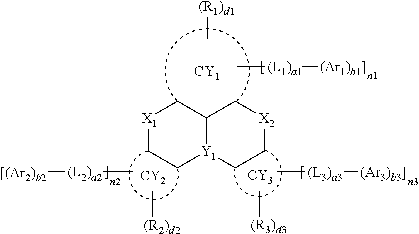
wherein, in Formula 1,
CY₁ to CY₃ are each independently a C₅-C₆₀ carbocyclic group or a C₁-C₆₀ heterocyclic group,
X₁ is C(R₄)(R₅), N(R₄), O, or S,
X₂ is C(R₆)(R₇), N(R₆), O, or S,
Y₁ is N, B, P, or P(═O),
L₁ to L₃ are each independently a single bond, a C₅-C₆₀ carbocyclic group unsubstituted or substituted with at least one R₁₀ₐ, or a C₁-C₆₀ heterocyclic group unsubstituted or substituted with at least one R₁₀ₐ,
a1 to a3 are each independently an integer from 1 to 5,
Ar₁ to Ar₃ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
b1 to b3 are each independently an integer from 1 to 12,
n1 to n3 are each independently an integer from 0 to 5,
a sum of n1, n2, and n3 is 1 or more,
R₁ to R₇ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
d1 to d3 are each independently an integer from 1 to 12,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, or any combination thereof.

2. The light-emitting device of claim 1, wherein the first electrode is an anode,
the second electrode is a cathode,
the interlayer further comprises an electron transport region between the emission layer and the second electrode,
the hole transport region further comprises a hole injection layer, an emission auxiliary layer, an electron blocking layer, or any combination thereof, and
the electron transport region comprises a buffer layer, a hole-blocking layer, an electron control layer, an electron transport layer, an electron injection layer, or any combination thereof.

3. The light-emitting device of claim 1, wherein the inorganic material comprises WOₓ, MoOₓ, GaOₓ, NiO_{y}, CuO_{y}, or any combination thereof, wherein x is a real number satisfying 2.5≤x≤3.0, and y is a real number satisfying 0.5≤y≤1.0.

4. The light-emitting device of claim 1, wherein the hole transport layer does not comprise a p-dopant.

5. The light-emitting device of claim 1, wherein the first electrode is in direct contact with the hole transport layer.

6. The light-emitting device of claim 1, wherein Ar₁ to Ar₃ are each independently a benzene group, a naphthalene group, an anthracene group, a phenanthrene group, a triphenylene group, a pyrene group, a chrysene group, a cyclopentadiene group, a 1,2,3,4-tetrahydronaphthalene group, a 9,9′-spirobifluorene group, a spiro[cyclohexane-1,9′-fluorene]group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophenegroup, a dibenzofuran group, a dibenzothiophene 5-oxide group, a 9H-fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluoren-9-one group, an azadibenzothiophene 5,5-dioxide group, an indolocarbazole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isooxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group, each unsubstituted or substituted with at least one R₁₀ₐ, and
R₁₀ₐ is as described in claim 1.

7. The light-emitting device of claim 1, wherein Ar₁ to Ar₃ are each independently represented by one of Formulas 2-1 to 2-36: 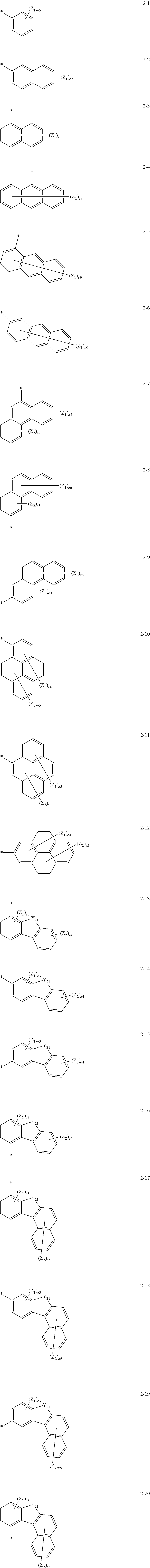 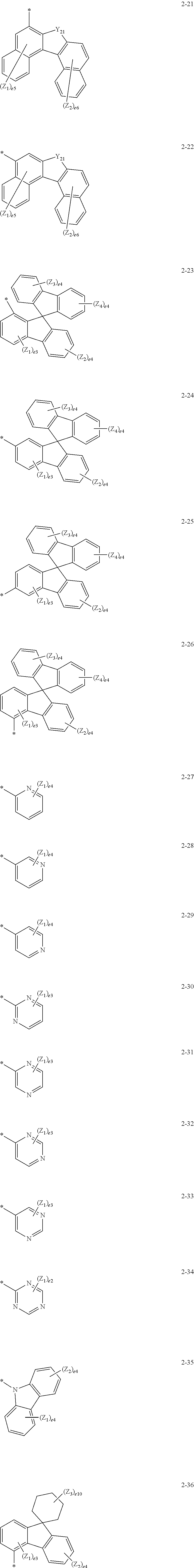
wherein, in Formulas 2-1 to 2-36,
Y₂₁ is O, S, N(Z₅), C(Z₅)(Z₆), or Si(Z₅)(Z₆),
Z₁ to Z₆ are each independently hydrogen, deuterium, —F, —Cl, —Br, —I, a hydroxyl group, a cyano group, a nitro group, an amidino group, a hydrazino group, a hydrazono group, —CF₃, —CF₂H, —CFH₂, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a naphthyl group, a fluorenyl group, a spiro-bifluorenyl group, a spiro-fluorene-benzofluorenyl group, a benzofluorenyl group, a dibenzofluorenyl group, a phenalenyl group, a phenanthrenyl group, an anthracenyl group, a fluoranthenyl group, a triphenylenyl group, a pyridinyl group, a pyrimidinyl group, a pyrazinyl group, a triazinyl group, a quinolinyl group, an isoquinolinyl group, a benzoquinolinyl group, a naphthyridinyl group, a quinoxalinyl group, a quinazolinyl group, a phenanthridinyl group, an acridinyl group, a phenanthrolinyl group, a phenazinyl group, a carbazolyl group, a dibenzofuranyl group, a dibenzothiophenyl group, a dibenzosilolyl group, —Si(Q₃₁)(Q₃₂)(Q₃₃), —N(Q₃₁)(Q₃₂), or —B(Q₃₁)(Q₃₂),
e2 is 1 or 2,
e3 is an integer from 1 to 3,
e4 is an integer from 1 to 4,
e5 is an integer from 1 to 5,
e6 is an integer from 1 to 6,
e7 is an integer from 1 to 7,
e9 is an integer from 1 to 9,
e10 is an integer from 1 to 10,
Q₃₁ to Q₃₃ are each independently a C₁-C₁₀ alkyl group, a C₁-C₁₀ alkoxy group, a phenyl group, a biphenyl group, a terphenyl group, or a naphthyl group, and
* indicates a binding site to a neighboring atom.

8. The light-emitting device of claim 1, wherein the at least one condensed cyclic compound represented by Formula 1 is represented by one of Formulas 1-1 to 1-5: 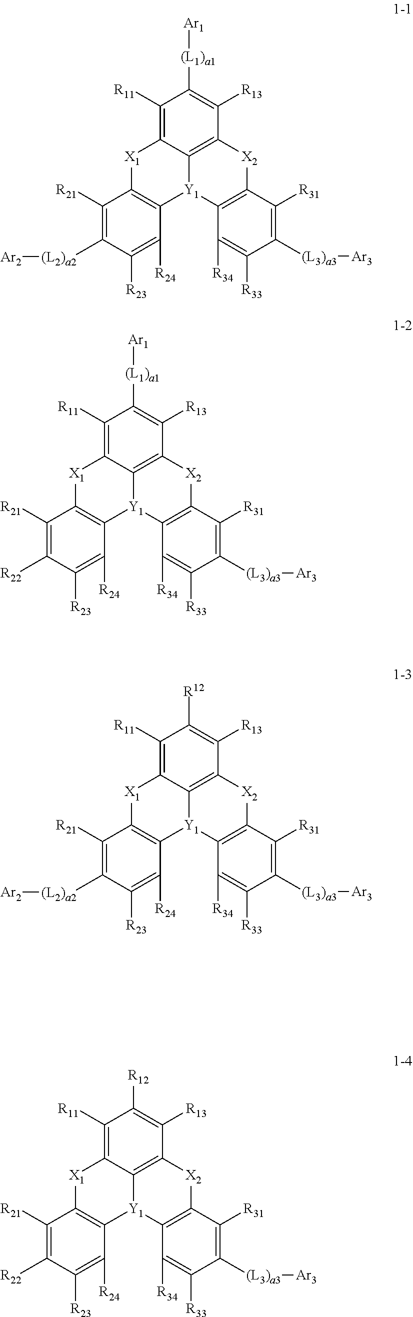 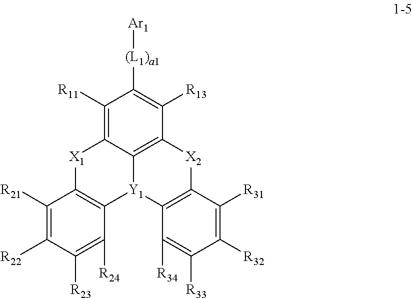
wherein, in Formulas 1-1 to 1-5,
X₁, X₂, Y₁, L₁ to L₃, a1 to a3, and Ar₁ to Ar₃ are respectively as described in claim 1,
R₁₁ to R₁₃ are respectively as described in connection with R₁ in claim 1,
R₂₁ to R₂₄ are respectively as described in connection with R₂ in claim 1, and
R₃₁ to R₃₄ are respectively as described in connection with R₃ in claim 1.

9. The light-emitting device of claim 1, wherein the at least one condensed cyclic compound represented by Formula 1 is one of Compounds 1 to 140:

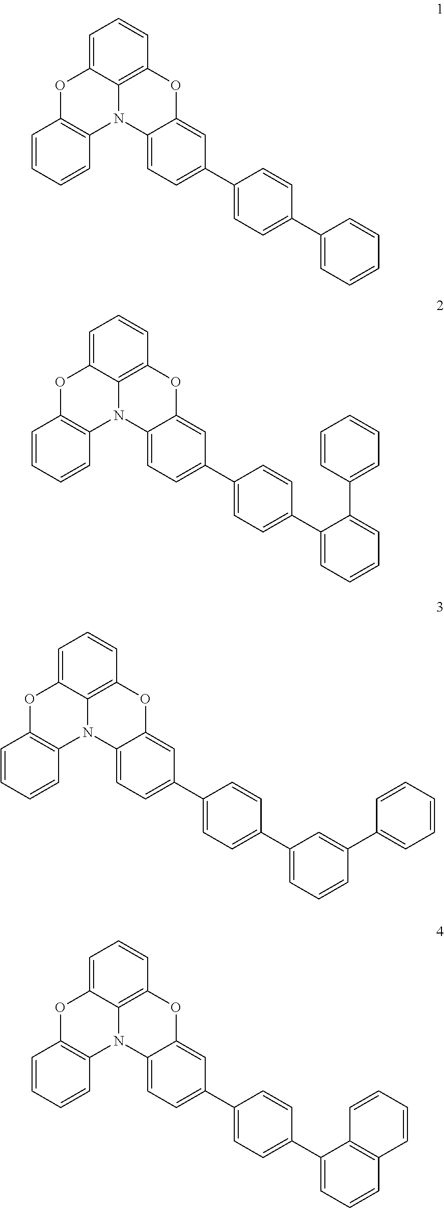 [SMILES: c1ccc(-c2ccc(-c3ccc4c(c3)Oc3cccc5c3N4c3ccccc3O5)cc2)cc1 ; c1ccc(-c2ccccc2-c2ccc(-c3ccc4c(c3)Oc3cccc5c3N4c3ccccc3O5)cc2)cc1 ; c1ccc(-c2cccc(-c3ccc(-c4ccc5c(c4)Oc4cccc6c4N5c4ccccc4O6)cc3)c2)cc1 ; c1ccc2c(c1)Oc1cccc3c1N2c1ccc(-c2ccc(-c4cccc5ccccc45)cc2)cc1O3]

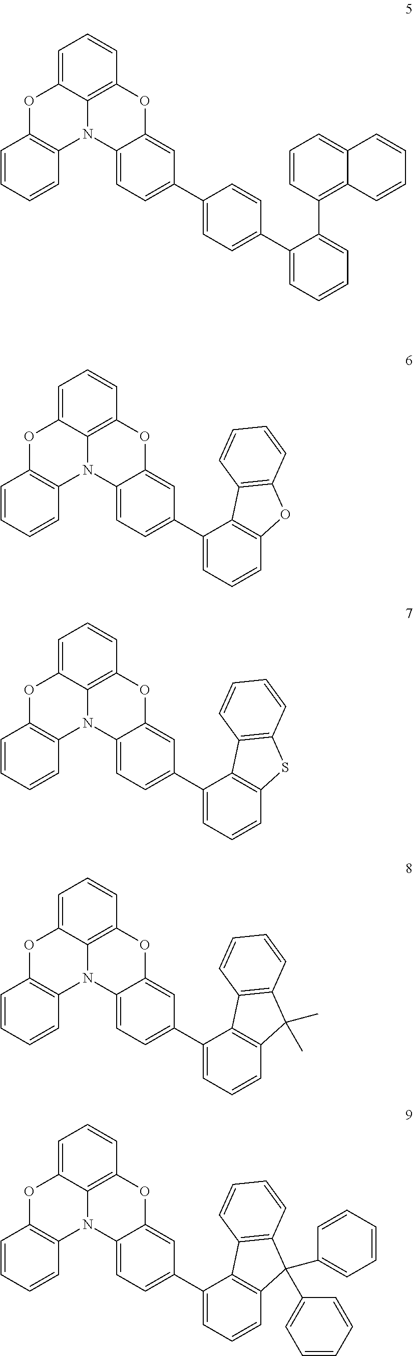 [SMILES: c1ccc2c(c1)Oc1cccc3c1N2c1ccc(-c2ccc(-c4ccccc4-c4cccc5ccccc45)cc2)cc1O3 ; c1ccc2c(c1)Oc1cccc3c1N2c1ccc(-c2cccc4oc5ccccc5c24)cc1O3 ; c1ccc2c(c1)Oc1cccc3c1N2c1ccc(-c2cccc4sc5ccccc5c24)cc1O3 ; CC1(C)c2ccccc2-c2c(-c3ccc4c(c3)Oc3cccc5c3N4c3ccccc3O5)cccc21 ; c1ccc(C2(c3ccccc3)c3ccccc3-c3c(-c4ccc5c(c4)Oc4cccc6c4N5c4ccccc4O6)cccc32)cc1]

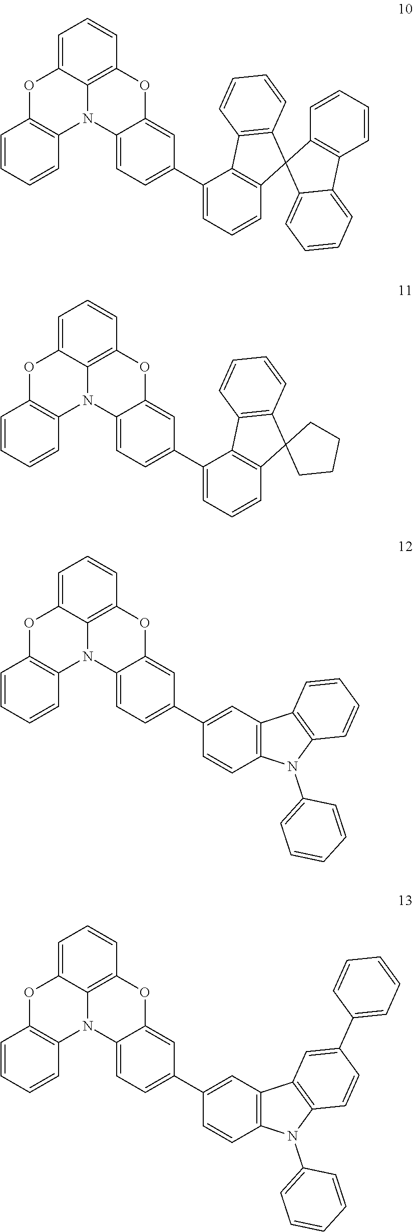 [SMILES: c1ccc2c(c1)Oc1cccc3c1N2c1ccc(-c2cccc4c2-c2ccccc2C42c4ccccc4-c4ccccc42)cc1O3 ; c1ccc2c(c1)Oc1cccc3c1N2c1ccc(-c2cccc4c2-c2ccccc2C42CCCC2)cc1O3 ; c1ccc(-n2c3ccccc3c3cc(-c4ccc5c(c4)Oc4cccc6c4N5c4ccccc4O6)ccc32)cc1 ; c1ccc(-c2ccc3c(c2)c2cc(-c4ccc5c(c4)Oc4cccc6c4N5c4ccccc4O6)ccc2n3-c2ccccc2)cc1]

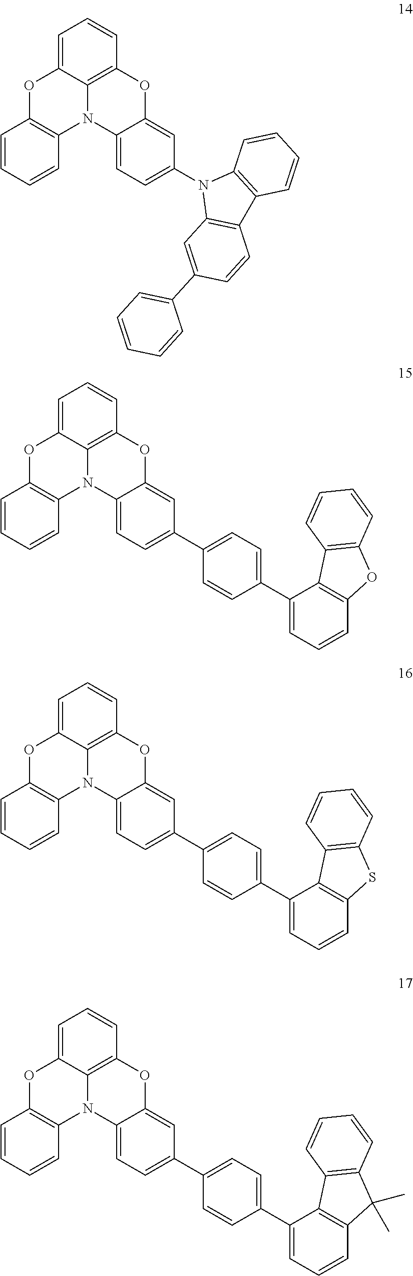 [SMILES: c1ccc(-c2ccc3c4ccccc4n(-c4ccc5c(c4)Oc4cccc6c4N5c4ccccc4O6)c3c2)cc1 ; c1ccc2c(c1)Oc1cccc3c1N2c1ccc(-c2ccc(-c4cccc5oc6ccccc6c45)cc2)cc1O3 ; c1ccc2c(c1)Oc1cccc3c1N2c1ccc(-c2ccc(-c4cccc5sc6ccccc6c45)cc2)cc1O3 ; CC1(C)c2ccccc2-c2c(-c3ccc(-c4ccc5c(c4)Oc4cccc6c4N5c4ccccc4O6)cc3)cccc21]

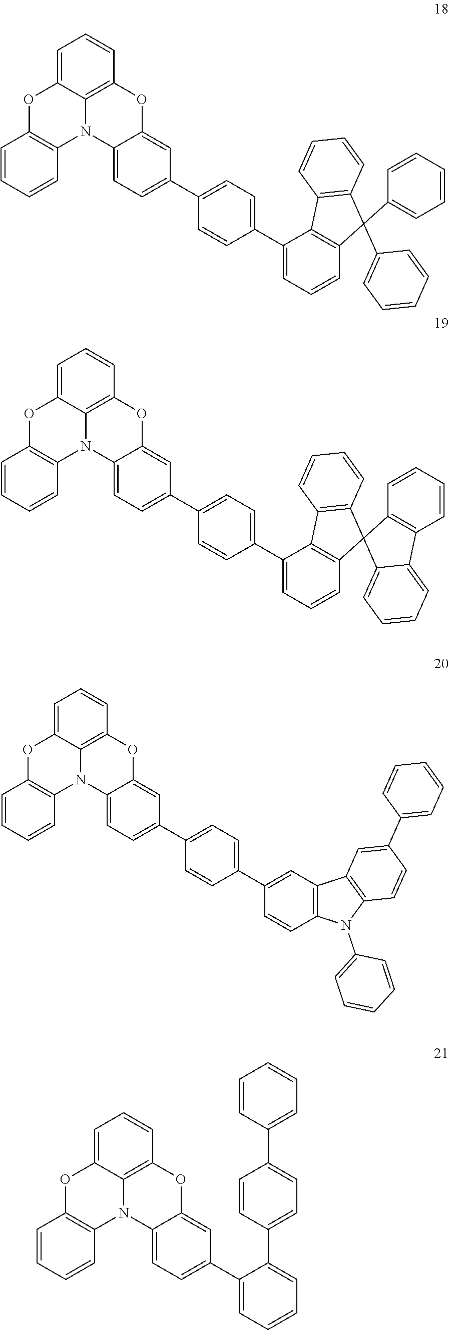 [SMILES: c1ccc(C2(c3ccccc3)c3ccccc3-c3c(-c4ccc(-c5ccc6c(c5)Oc5cccc7c5N6c5ccccc5O7)cc4)cccc32)cc1 ; c1ccc2c(c1)Oc1cccc3c1N2c1ccc(-c2ccc(-c4cccc5c4-c4ccccc4C54c5ccccc5-c5ccccc54)cc2)cc1O3 ; c1ccc(-c2ccc3c(c2)c2cc(-c4ccc(-c5ccc6c(c5)Oc5cccc7c5N6c5ccccc5O7)cc4)ccc2n3-c2ccccc2)cc1 ; c1ccc(-c2ccc(-c3ccccc3-c3ccc4c(c3)Oc3cccc5c3N4c3ccccc3O5)cc2)cc1]

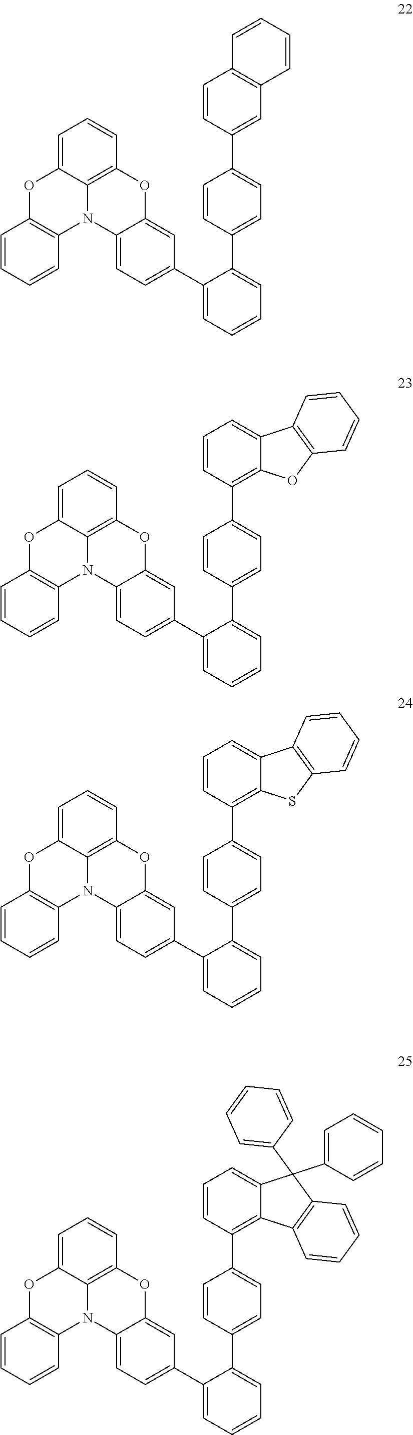 [SMILES: c1ccc2c(c1)Oc1cccc3c1N2c1ccc(-c2ccccc2-c2ccc(-c4ccc5ccccc5c4)cc2)cc1O3 ; c1ccc2c(c1)Oc1cccc3c1N2c1ccc(-c2ccccc2-c2ccc(-c4cccc5c4oc4ccccc45)cc2)cc1O3 ; c1ccc2c(c1)Oc1cccc3c1N2c1ccc(-c2ccccc2-c2ccc(-c4cccc5c4sc4ccccc45)cc2)cc1O3 ; c1ccc(C2(c3ccccc3)c3ccccc3-c3c(-c4ccc(-c5ccccc5-c5ccc6c(c5)Oc5cccc7c5N6c5ccccc5O7)cc4)cccc32)cc1]

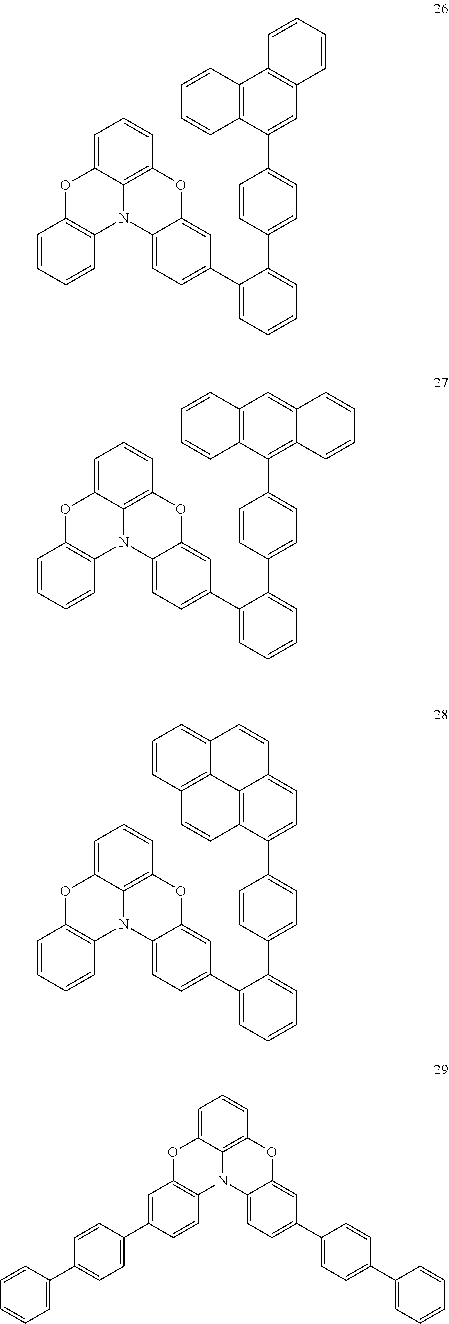 [SMILES: c1ccc2c(c1)Oc1cccc3c1N2c1ccc(-c2ccccc2-c2ccc(-c4cc5ccccc5c5ccccc45)cc2)cc1O3 ; c1ccc2c(c1)Oc1cccc3c1N2c1ccc(-c2ccccc2-c2ccc(-c4c5ccccc5cc5ccccc45)cc2)cc1O3 ; c1ccc2c(c1)Oc1cccc3c1N2c1ccc(-c2ccccc2-c2ccc(-c4ccc5ccc6cccc7ccc4c5c67)cc2)cc1O3 ; c1ccc(-c2ccc(-c3ccc4c(c3)Oc3cccc5c3N4c3ccc(-c4ccc(-c6ccccc6)cc4)cc3O5)cc2)cc1]

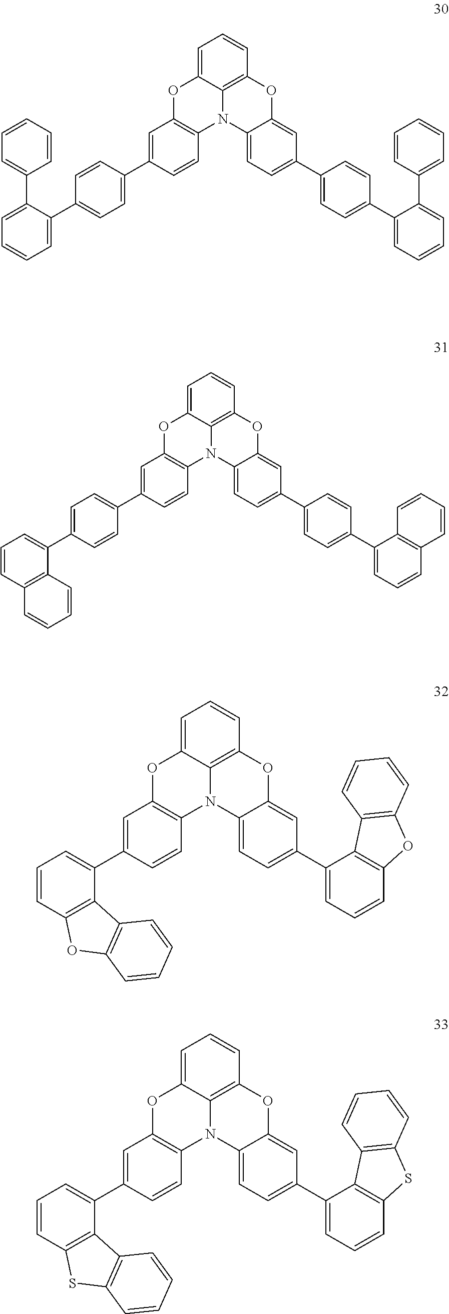 [SMILES: c1ccc(-c2ccccc2-c2ccc(-c3ccc4c(c3)Oc3cccc5c3N4c3ccc(-c4ccc(-c6ccccc6-c6ccccc6)cc4)cc3O5)cc2)cc1 ; c1cc2c3c(c1)Oc1cc(-c4ccc(-c5cccc6ccccc56)cc4)ccc1N3c1ccc(-c3ccc(-c4cccc5ccccc45)cc3)cc1O2 ; c1cc2c3c(c1)Oc1cc(-c4cccc5oc6ccccc6c45)ccc1N3c1ccc(-c3cccc4oc5ccccc5c34)cc1O2 ; c1cc2c3c(c1)Oc1cc(-c4cccc5sc6ccccc6c45)ccc1N3c1ccc(-c3cccc4sc5ccccc5c34)cc1O2]

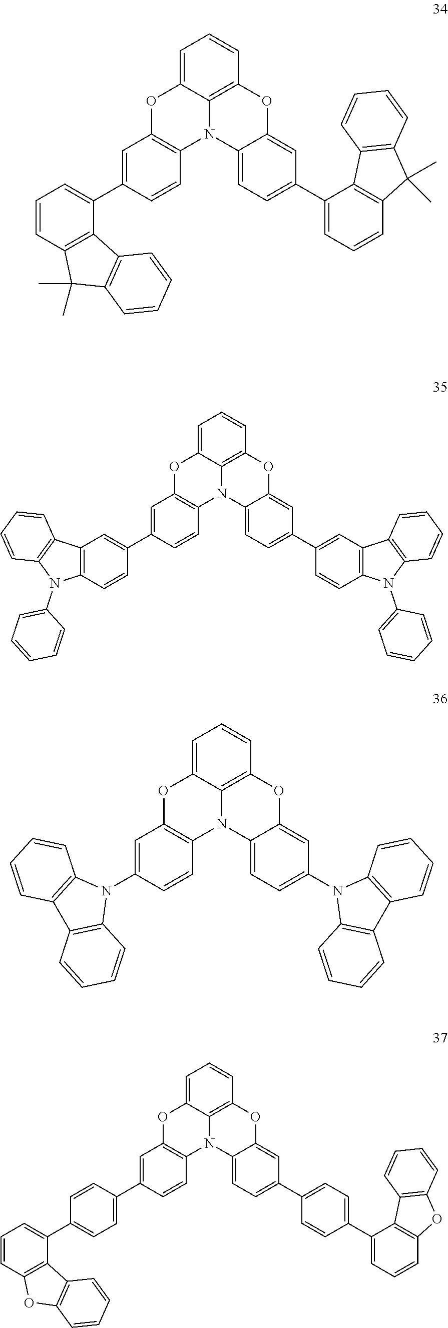 [SMILES: CC1(C)c2ccccc2-c2c(-c3ccc4c(c3)Oc3cccc5c3N4c3ccc(-c4cccc6c4-c4ccccc4C6(C)C)cc3O5)cccc21 ; c1ccc(-n2c3ccccc3c3cc(-c4ccc5c(c4)Oc4cccc6c4N5c4ccc(-c5ccc7c(c5)c5ccccc5n7-c5ccccc5)cc4O6)ccc32)cc1 ; c1cc2c3c(c1)Oc1cc(-n4c5ccccc5c5ccccc54)ccc1N3c1ccc(-n3c4ccccc4c4ccccc43)cc1O2 ; c1cc2c3c(c1)Oc1cc(-c4ccc(-c5cccc6oc7ccccc7c56)cc4)ccc1N3c1ccc(-c3ccc(-c4cccc5oc6ccccc6c45)cc3)cc1O2]

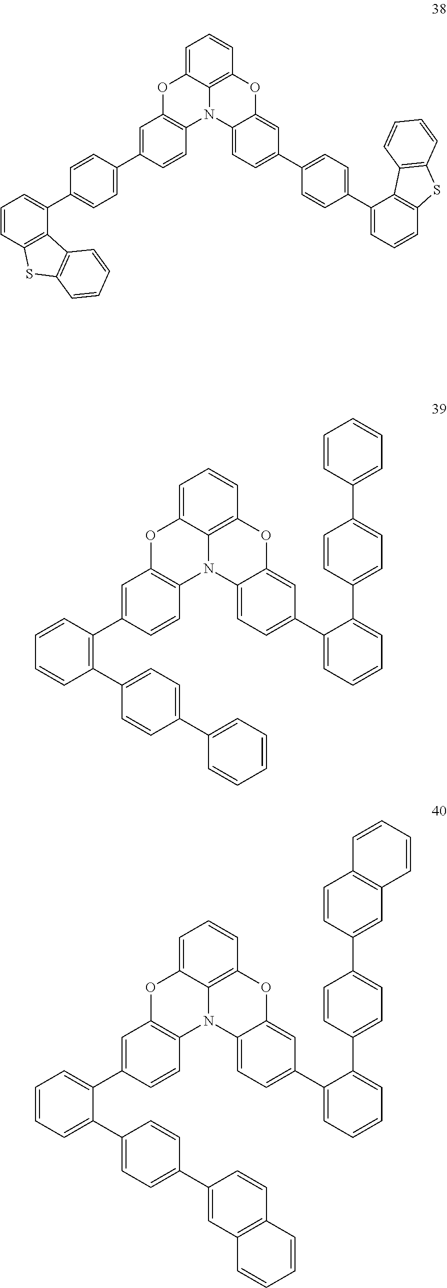 [SMILES: c1cc2c3c(c1)Oc1cc(-c4ccc(-c5cccc6sc7ccccc7c56)cc4)ccc1N3c1ccc(-c3ccc(-c4cccc5sc6ccccc6c45)cc3)cc1O2 ; c1ccc(-c2ccc(-c3ccccc3-c3ccc4c(c3)Oc3cccc5c3N4c3ccc(-c4ccccc4-c4ccc(-c6ccccc6)cc4)cc3O5)cc2)cc1 ; c1cc2c3c(c1)Oc1cc(-c4ccccc4-c4ccc(-c5ccc6ccccc6c5)cc4)ccc1N3c1ccc(-c3ccccc3-c3ccc(-c4ccc5ccccc5c4)cc3)cc1O2]

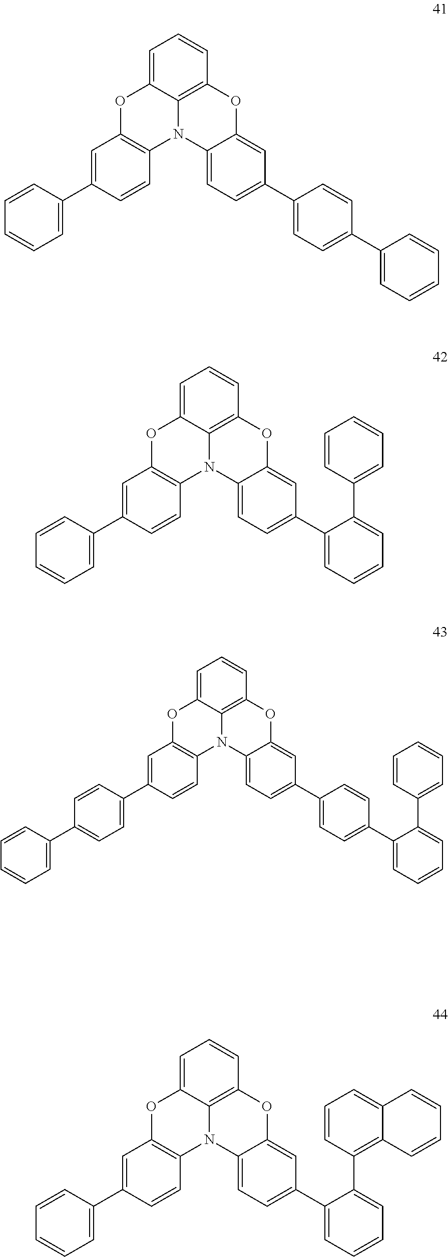 [SMILES: c1ccc(-c2ccc(-c3ccc4c(c3)Oc3cccc5c3N4c3ccc(-c4ccccc4)cc3O5)cc2)cc1 ; c1ccc(-c2ccc3c(c2)Oc2cccc4c2N3c2ccc(-c3ccccc3-c3ccccc3)cc2O4)cc1 ; c1ccc(-c2ccc(-c3ccc4c(c3)Oc3cccc5c3N4c3ccc(-c4ccc(-c6ccccc6-c6ccccc6)cc4)cc3O5)cc2)cc1 ; c1ccc(-c2ccc3c(c2)Oc2cccc4c2N3c2ccc(-c3ccccc3-c3cccc5ccccc35)cc2O4)cc1]

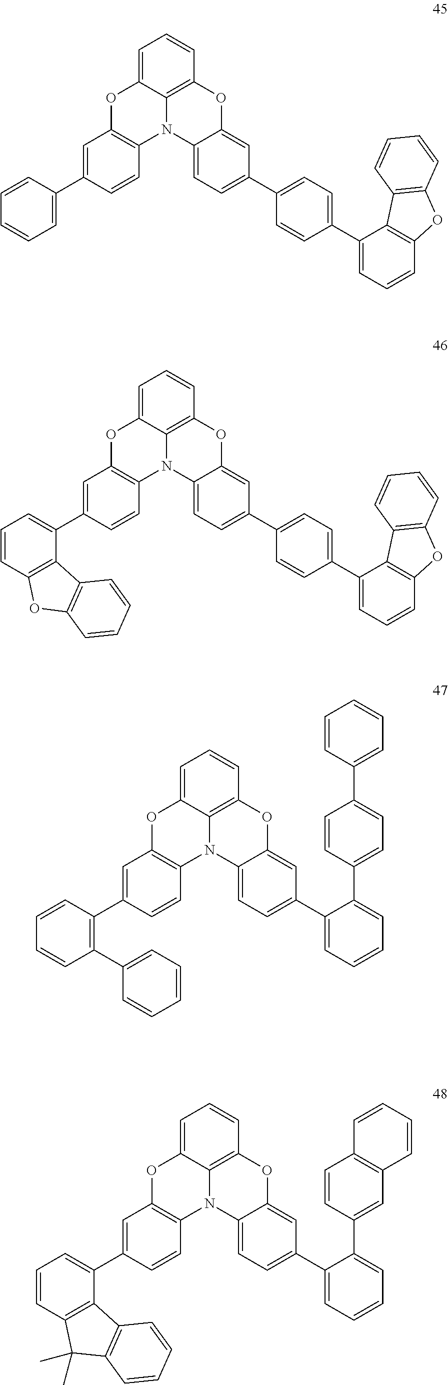 [SMILES: c1ccc(-c2ccc3c(c2)Oc2cccc4c2N3c2ccc(-c3ccc(-c5cccc6oc7ccccc7c56)cc3)cc2O4)cc1 ; c1cc2c3c(c1)Oc1cc(-c4cccc5oc6ccccc6c45)ccc1N3c1ccc(-c3ccc(-c4cccc5oc6ccccc6c45)cc3)cc1O2 ; c1ccc(-c2ccc(-c3ccccc3-c3ccc4c(c3)Oc3cccc5c3N4c3ccc(-c4ccccc4-c4ccccc4)cc3O5)cc2)cc1 ; CC1(C)c2ccccc2-c2c(-c3ccc4c(c3)Oc3cccc5c3N4c3ccc(-c4ccccc4-c4ccc6ccccc6c4)cc3O5)cccc21]

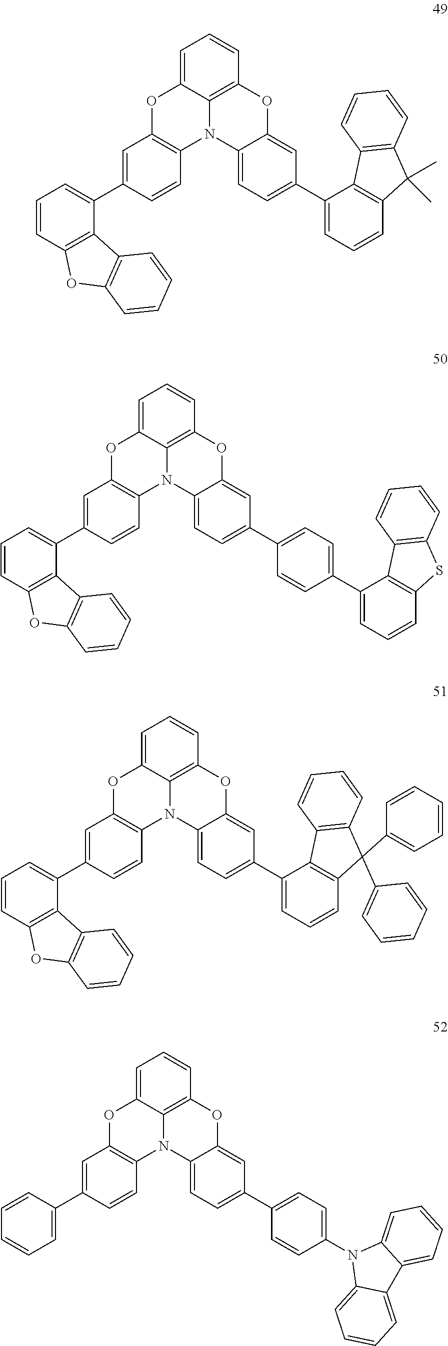 [SMILES: CC1(C)c2ccccc2-c2c(-c3ccc4c(c3)Oc3cccc5c3N4c3ccc(-c4cccc6oc7ccccc7c46)cc3O5)cccc21 ; c1cc2c3c(c1)Oc1cc(-c4cccc5oc6ccccc6c45)ccc1N3c1ccc(-c3ccc(-c4cccc5sc6ccccc6c45)cc3)cc1O2 ; c1ccc(C2(c3ccccc3)c3ccccc3-c3c(-c4ccc5c(c4)Oc4cccc6c4N5c4ccc(-c5cccc7oc8ccccc8c57)cc4O6)cccc32)cc1 ; c1ccc(-c2ccc3c(c2)Oc2cccc4c2N3c2ccc(-c3ccc(-n5c6ccccc6c6ccccc65)cc3)cc2O4)cc1]

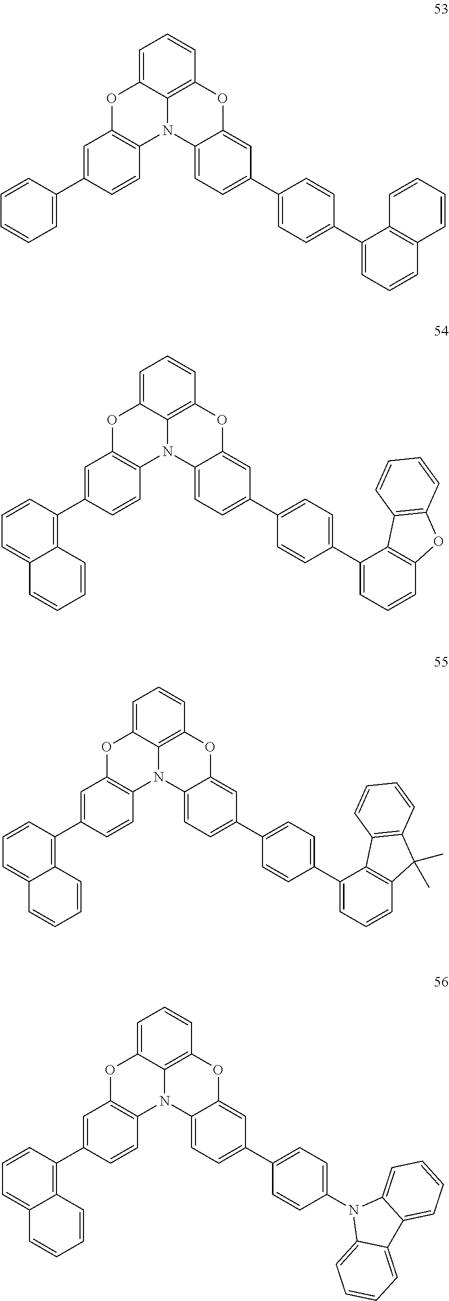 [SMILES: c1ccc(-c2ccc3c(c2)Oc2cccc4c2N3c2ccc(-c3ccc(-c5cccc6ccccc56)cc3)cc2O4)cc1 ; c1cc2c3c(c1)Oc1cc(-c4cccc5ccccc45)ccc1N3c1ccc(-c3ccc(-c4cccc5oc6ccccc6c45)cc3)cc1O2 ; CC1(C)c2ccccc2-c2c(-c3ccc(-c4ccc5c(c4)Oc4cccc6c4N5c4ccc(-c5cccc7ccccc57)cc4O6)cc3)cccc21 ; c1cc2c3c(c1)Oc1cc(-c4cccc5ccccc45)ccc1N3c1ccc(-c3ccc(-n4c5ccccc5c5ccccc54)cc3)cc1O2]

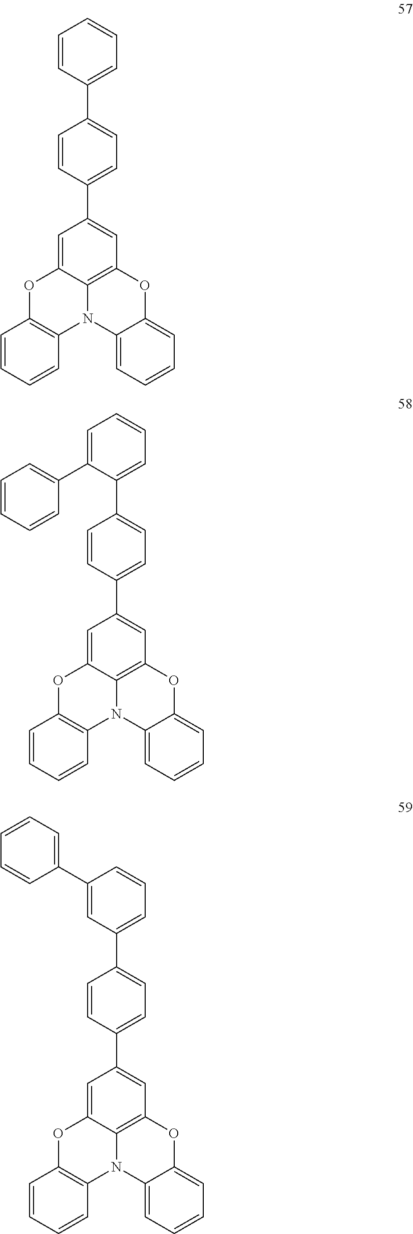

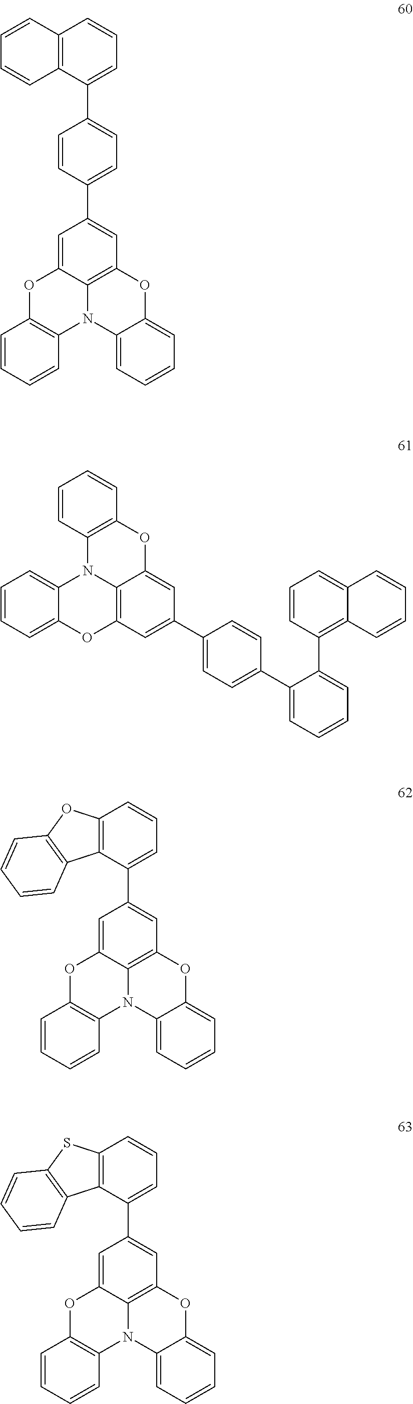 [SMILES: c1ccc2c(c1)Oc1cc(-c3ccc(-c4cccc5ccccc45)cc3)cc3c1N2c1ccccc1O3 ; c1ccc2c(c1)Oc1cc(-c3ccc(-c4ccccc4-c4cccc5ccccc45)cc3)cc3c1N2c1ccccc1O3 ; c1ccc2c(c1)Oc1cc(-c3cccc4oc5ccccc5c34)cc3c1N2c1ccccc1O3 ; c1ccc2c(c1)Oc1cc(-c3cccc4sc5ccccc5c34)cc3c1N2c1ccccc1O3]

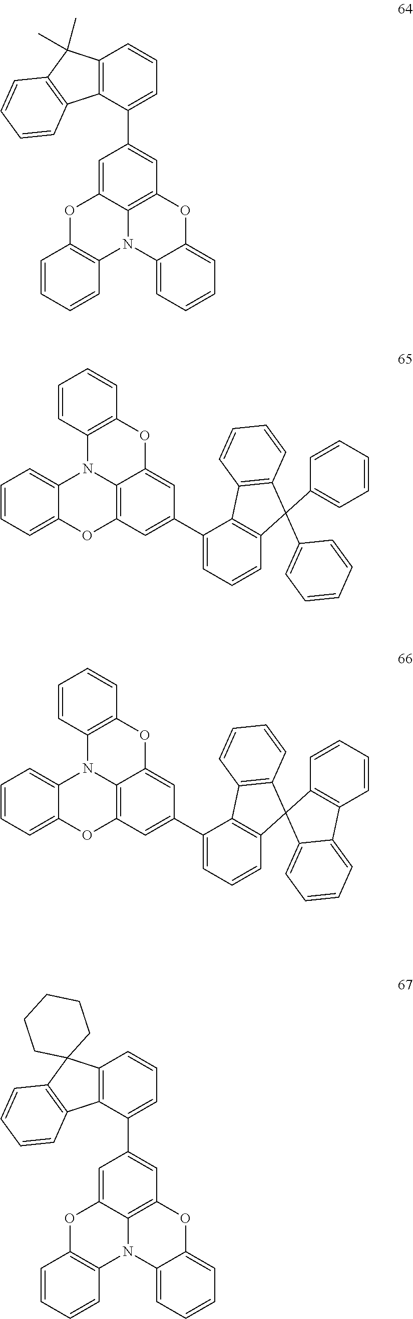 [SMILES: CC1(C)c2ccccc2-c2c(-c3cc4c5c(c3)Oc3ccccc3N5c3ccccc3O4)cccc21 ; c1ccc(C2(c3ccccc3)c3ccccc3-c3c(-c4cc5c6c(c4)Oc4ccccc4N6c4ccccc4O5)cccc32)cc1 ; c1ccc2c(c1)Oc1cc(-c3cccc4c3-c3ccccc3C43c4ccccc4-c4ccccc43)cc3c1N2c1ccccc1O3 ; c1ccc2c(c1)Oc1cc(-c3cccc4c3-c3ccccc3C43CCCCC3)cc3c1N2c1ccccc1O3]

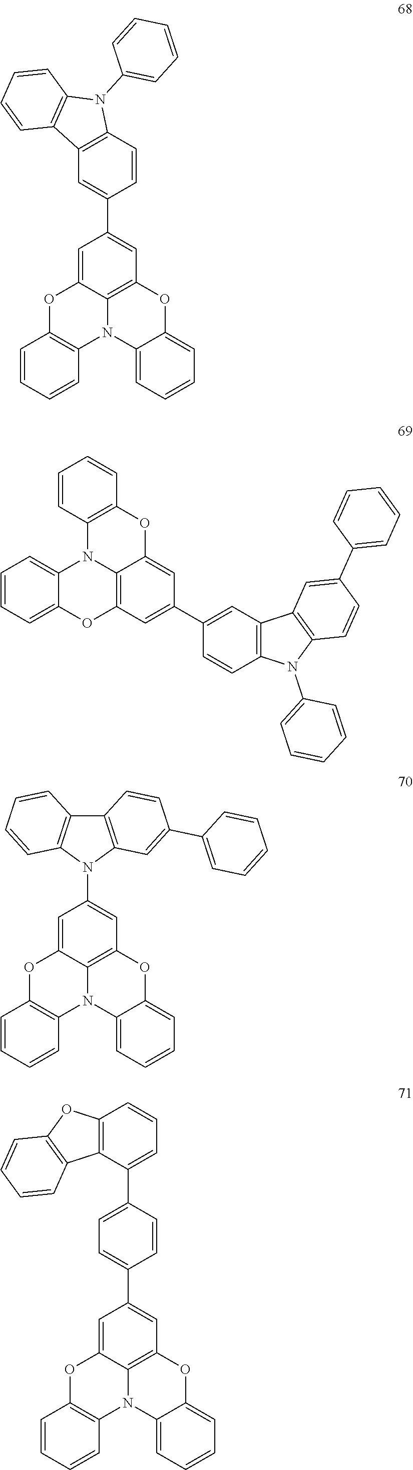

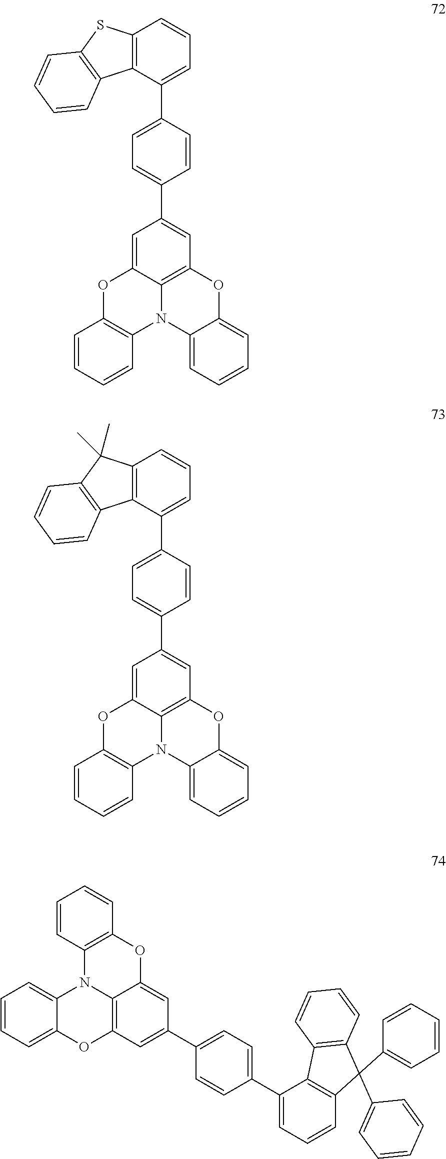

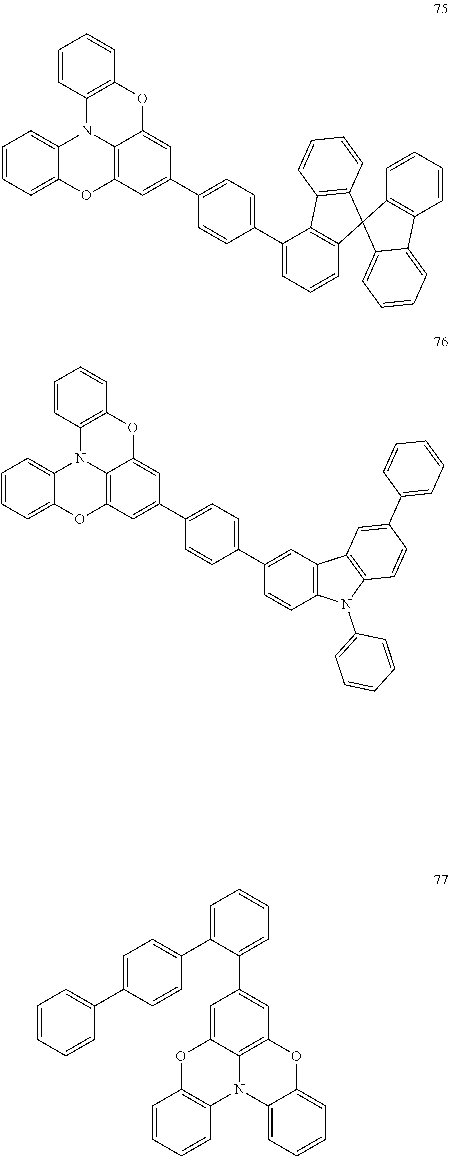 [SMILES: c1ccc2c(c1)Oc1cc(-c3ccc(-c4cccc5c4-c4ccccc4C54c5ccccc5-c5ccccc54)cc3)cc3c1N2c1ccccc1O3 ; c1ccc(-c2ccc3c(c2)c2cc(-c4ccc(-c5cc6c7c(c5)Oc5ccccc5N7c5ccccc5O6)cc4)ccc2n3-c2ccccc2)cc1 ; c1ccc(-c2ccc(-c3ccccc3-c3cc4c5c(c3)Oc3ccccc3N5c3ccccc3O4)cc2)cc1]

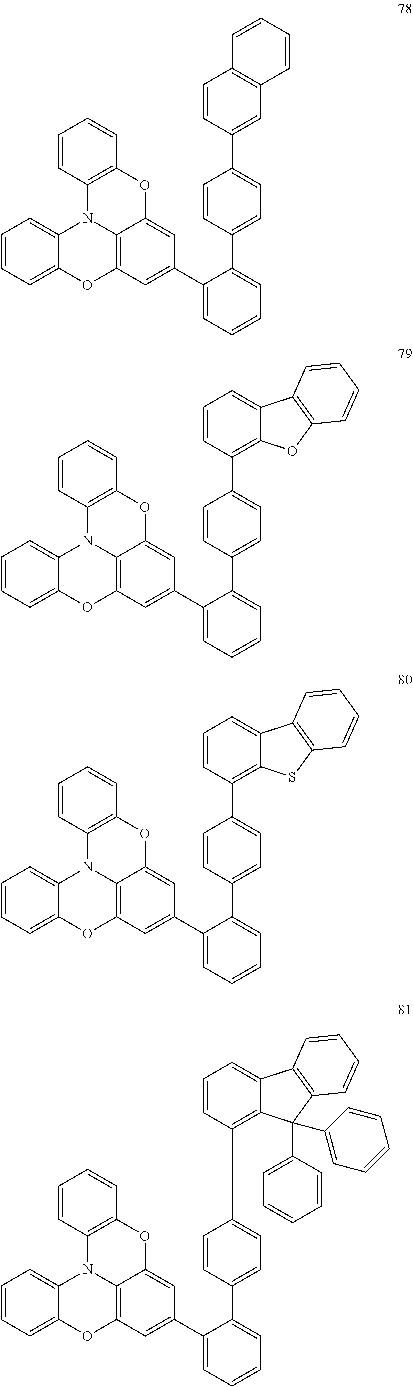 [SMILES: c1ccc2c(c1)Oc1cc(-c3ccccc3-c3ccc(-c4ccc5ccccc5c4)cc3)cc3c1N2c1ccccc1O3 ; c1ccc2c(c1)Oc1cc(-c3ccccc3-c3ccc(-c4cccc5c4oc4ccccc45)cc3)cc3c1N2c1ccccc1O3 ; c1ccc2c(c1)Oc1cc(-c3ccccc3-c3ccc(-c4cccc5c4sc4ccccc45)cc3)cc3c1N2c1ccccc1O3 ; c1ccc(C2(c3ccccc3)c3ccccc3-c3cccc(-c4ccc(-c5ccccc5-c5cc6c7c(c5)Oc5ccccc5N7c5ccccc5O6)cc4)c32)cc1]

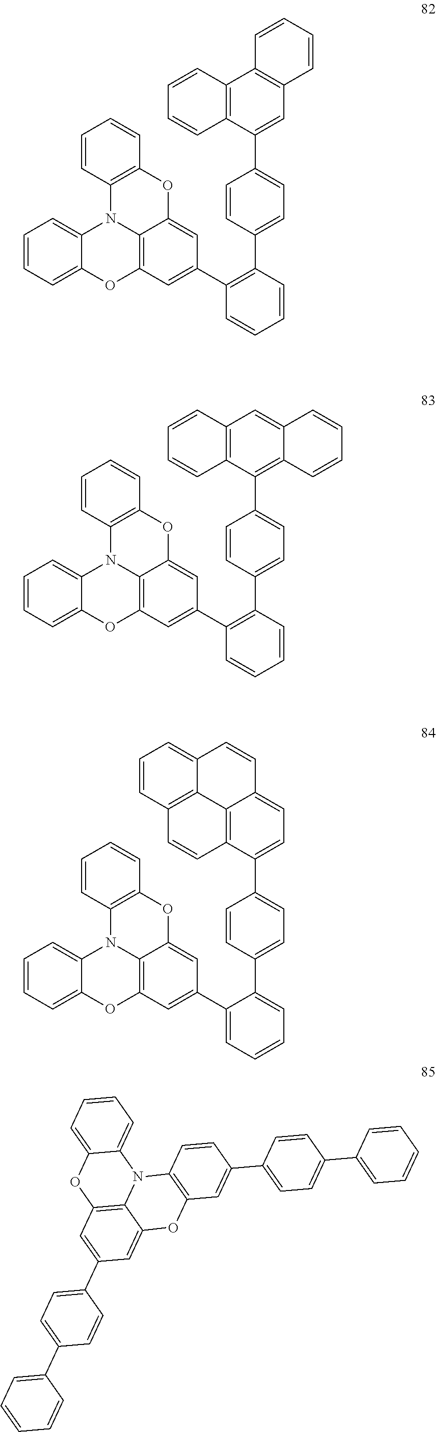 [SMILES: c1ccc2c(c1)Oc1cc(-c3ccccc3-c3ccc(-c4cc5ccccc5c5ccccc45)cc3)cc3c1N2c1ccccc1O3 ; c1ccc2c(c1)Oc1cc(-c3ccccc3-c3ccc(-c4c5ccccc5cc5ccccc45)cc3)cc3c1N2c1ccccc1O3 ; c1ccc2c(c1)Oc1cc(-c3ccccc3-c3ccc(-c4ccc5ccc6cccc7ccc4c5c67)cc3)cc3c1N2c1ccccc1O3 ; c1ccc(-c2ccc(-c3ccc4c(c3)Oc3cc(-c5ccc(-c6ccccc6)cc5)cc5c3N4c3ccccc3O5)cc2)cc1]

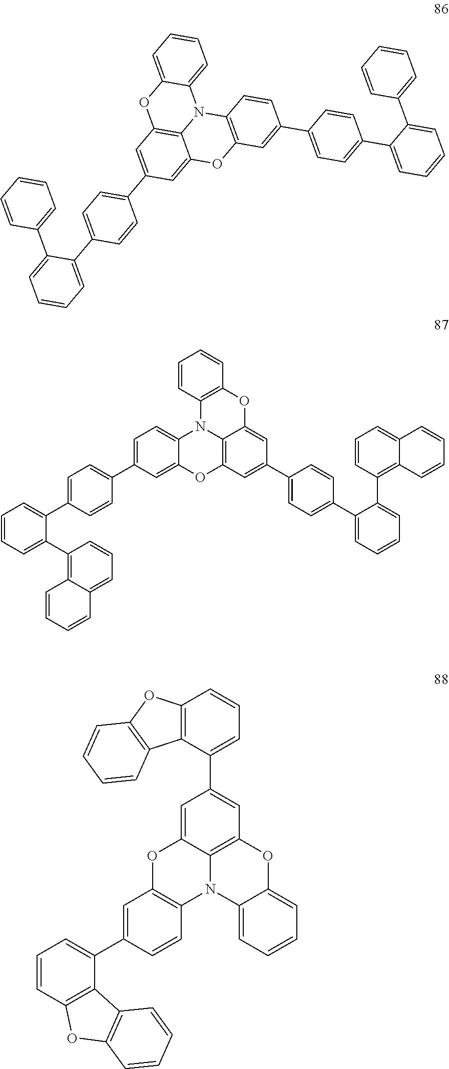 [SMILES: c1ccc(-c2ccccc2-c2ccc(-c3ccc4c(c3)Oc3cc(-c5ccc(-c6ccccc6-c6ccccc6)cc5)cc5c3N4c3ccccc3O5)cc2)cc1 ; c1ccc2c(c1)Oc1cc(-c3ccc(-c4ccccc4-c4cccc5ccccc45)cc3)cc3c1N2c1ccc(-c2ccc(-c4ccccc4-c4cccc5ccccc45)cc2)cc1O3 ; c1ccc2c(c1)Oc1cc(-c3cccc4oc5ccccc5c34)cc3c1N2c1ccc(-c2cccc4oc5ccccc5c24)cc1O3]

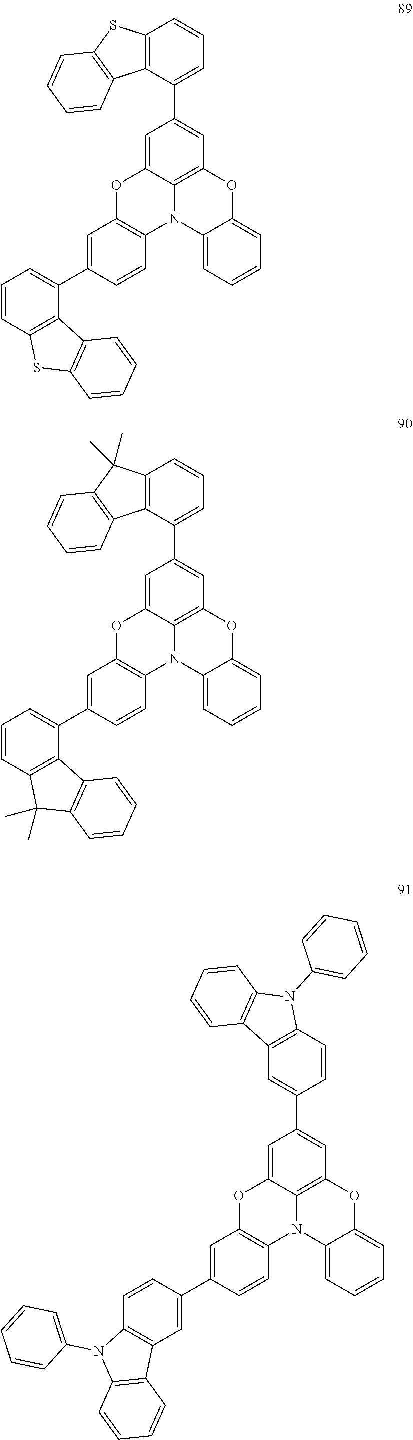

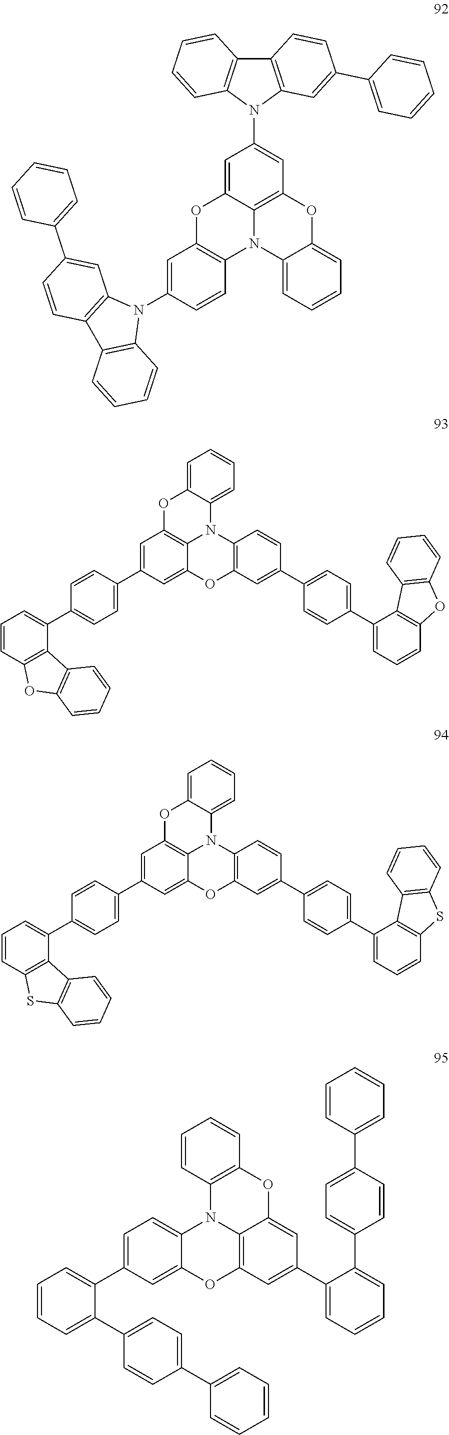 [SMILES: c1ccc(-c2ccc3c4ccccc4n(-c4ccc5c(c4)Oc4cc(-n6c7ccccc7c7ccc(-c8ccccc8)cc76)cc6c4N5c4ccccc4O6)c3c2)cc1 ; c1ccc2c(c1)Oc1cc(-c3ccc(-c4cccc5oc6ccccc6c45)cc3)cc3c1N2c1ccc(-c2ccc(-c4cccc5oc6ccccc6c45)cc2)cc1O3 ; c1ccc2c(c1)Oc1cc(-c3ccc(-c4cccc5sc6ccccc6c45)cc3)cc3c1N2c1ccc(-c2ccc(-c4cccc5sc6ccccc6c45)cc2)cc1O3 ; c1ccc(-c2ccc(-c3ccccc3-c3ccc4c(c3)Oc3cc(-c5ccccc5-c5ccc(-c6ccccc6)cc5)cc5c3N4c3ccccc3O5)cc2)cc1]

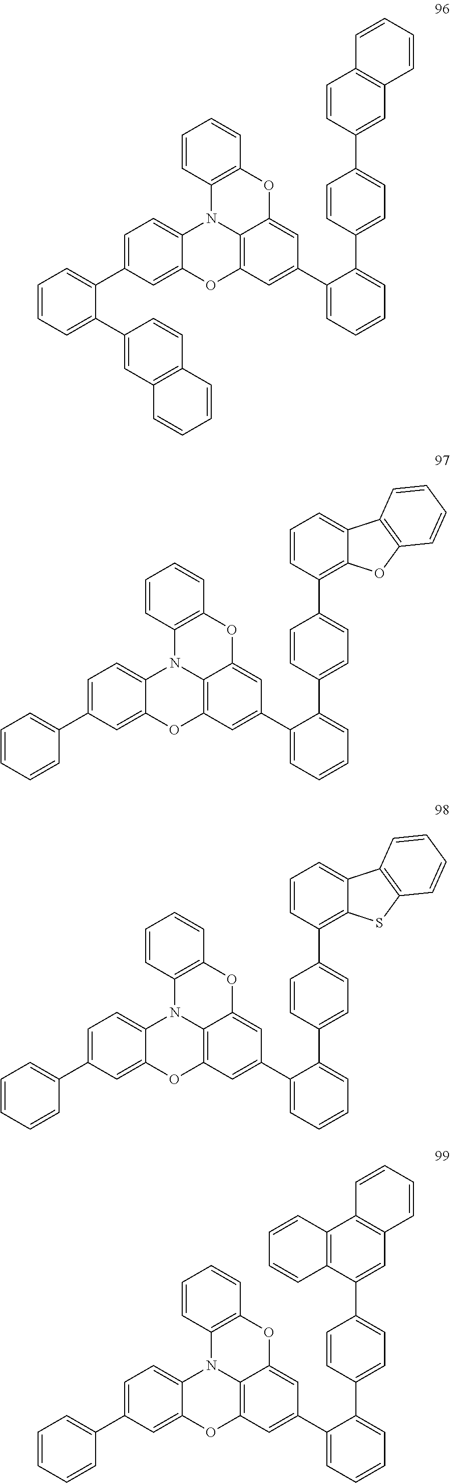 [SMILES: c1ccc2c(c1)Oc1cc(-c3ccccc3-c3ccc(-c4ccc5ccccc5c4)cc3)cc3c1N2c1ccc(-c2ccccc2-c2ccc4ccccc4c2)cc1O3 ; c1ccc(-c2ccc3c(c2)Oc2cc(-c4ccccc4-c4ccc(-c5cccc6c5oc5ccccc56)cc4)cc4c2N3c2ccccc2O4)cc1 ; c1ccc(-c2ccc3c(c2)Oc2cc(-c4ccccc4-c4ccc(-c5cccc6c5sc5ccccc56)cc4)cc4c2N3c2ccccc2O4)cc1 ; c1ccc(-c2ccc3c(c2)Oc2cc(-c4ccccc4-c4ccc(-c5cc6ccccc6c6ccccc56)cc4)cc4c2N3c2ccccc2O4)cc1]

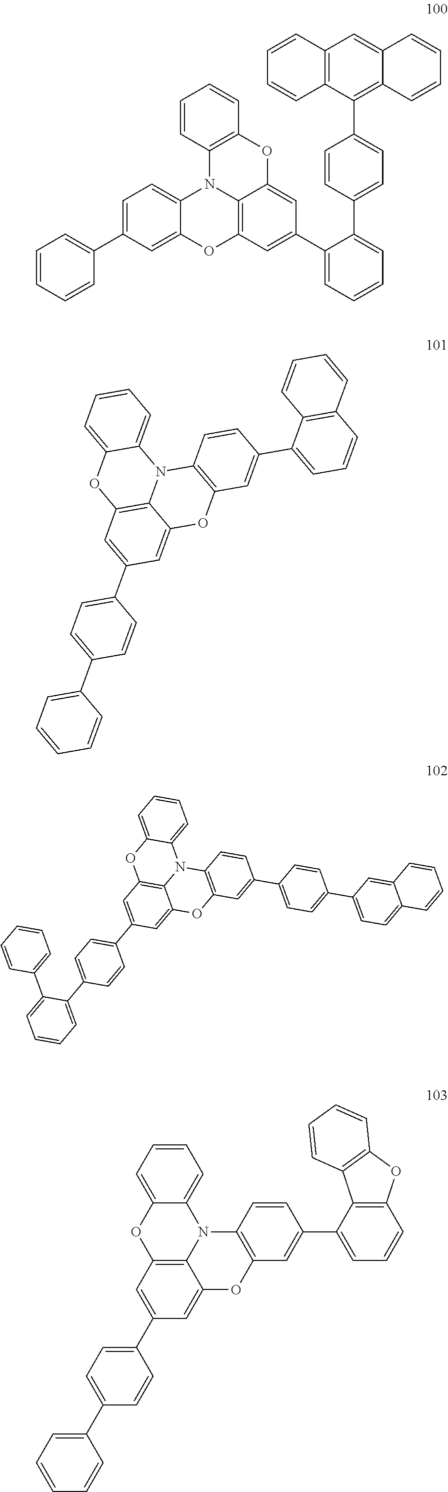 [SMILES: c1ccc(-c2ccc3c(c2)Oc2cc(-c4ccccc4-c4ccc(-c5c6ccccc6cc6ccccc56)cc4)cc4c2N3c2ccccc2O4)cc1 ; c1ccc(-c2ccc(-c3cc4c5c(c3)Oc3cc(-c6cccc7ccccc67)ccc3N5c3ccccc3O4)cc2)cc1 ; c1ccc(-c2ccccc2-c2ccc(-c3cc4c5c(c3)Oc3cc(-c6ccc(-c7ccc8ccccc8c7)cc6)ccc3N5c3ccccc3O4)cc2)cc1 ; c1ccc(-c2ccc(-c3cc4c5c(c3)Oc3cc(-c6cccc7oc8ccccc8c67)ccc3N5c3ccccc3O4)cc2)cc1]

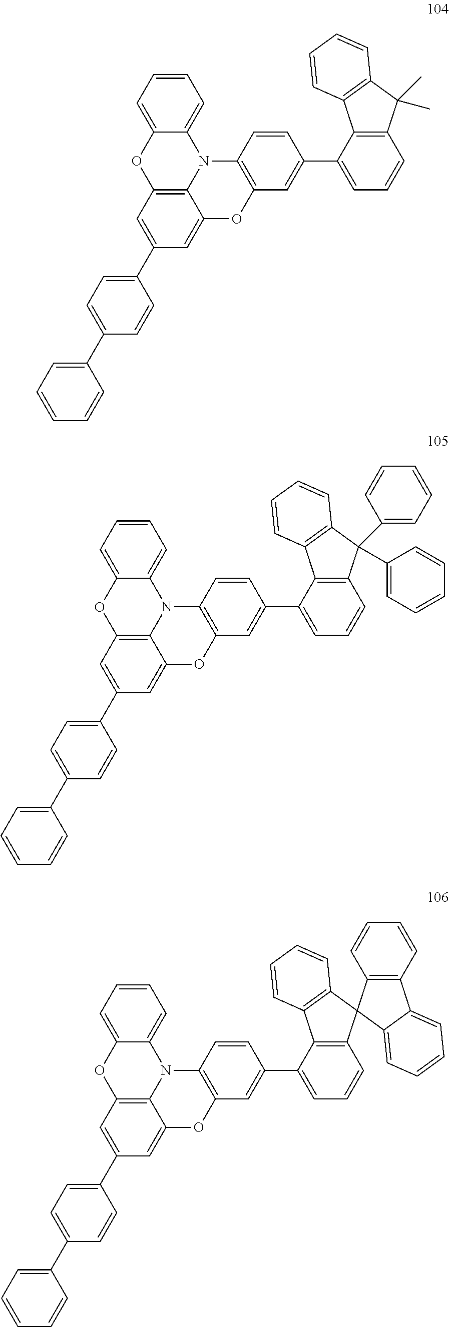 [SMILES: CC1(C)c2ccccc2-c2c(-c3ccc4c(c3)Oc3cc(-c5ccc(-c6ccccc6)cc5)cc5c3N4c3ccccc3O5)cccc21 ; c1ccc(-c2ccc(-c3cc4c5c(c3)Oc3cc(-c6cccc7c6-c6ccccc6C7(c6ccccc6)c6ccccc6)ccc3N5c3ccccc3O4)cc2)cc1 ; c1ccc(-c2ccc(-c3cc4c5c(c3)Oc3cc(-c6cccc7c6-c6ccccc6C76c7ccccc7-c7ccccc76)ccc3N5c3ccccc3O4)cc2)cc1]

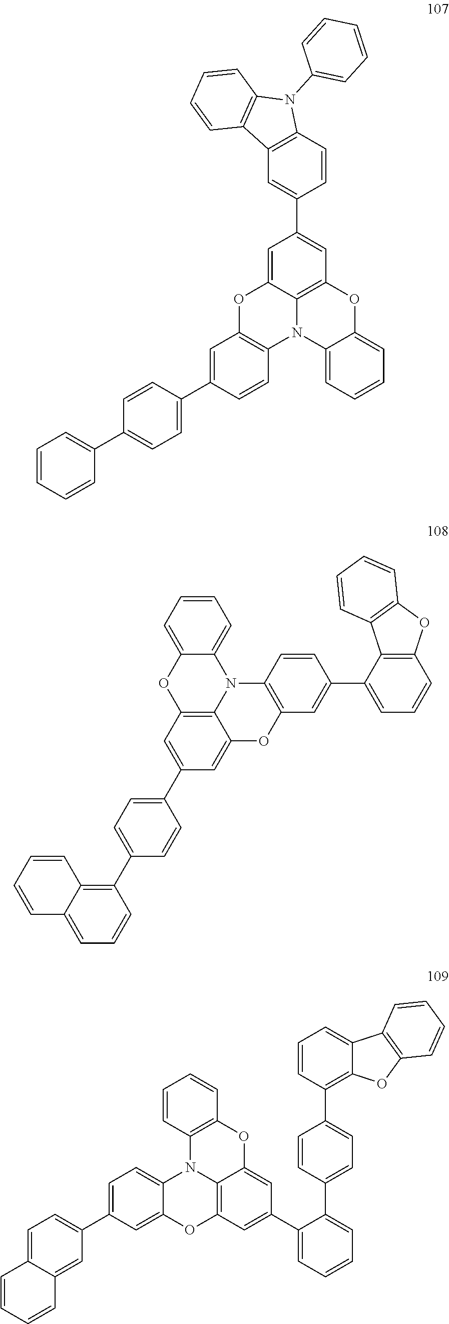 [SMILES: c1ccc(-c2ccc(-c3ccc4c(c3)Oc3cc(-c5ccc6c(c5)c5ccccc5n6-c5ccccc5)cc5c3N4c3ccccc3O5)cc2)cc1 ; c1ccc2c(c1)Oc1cc(-c3ccc(-c4cccc5ccccc45)cc3)cc3c1N2c1ccc(-c2cccc4oc5ccccc5c24)cc1O3 ; c1ccc2c(c1)Oc1cc(-c3ccccc3-c3ccc(-c4cccc5c4oc4ccccc45)cc3)cc3c1N2c1ccc(-c2ccc4ccccc4c2)cc1O3]

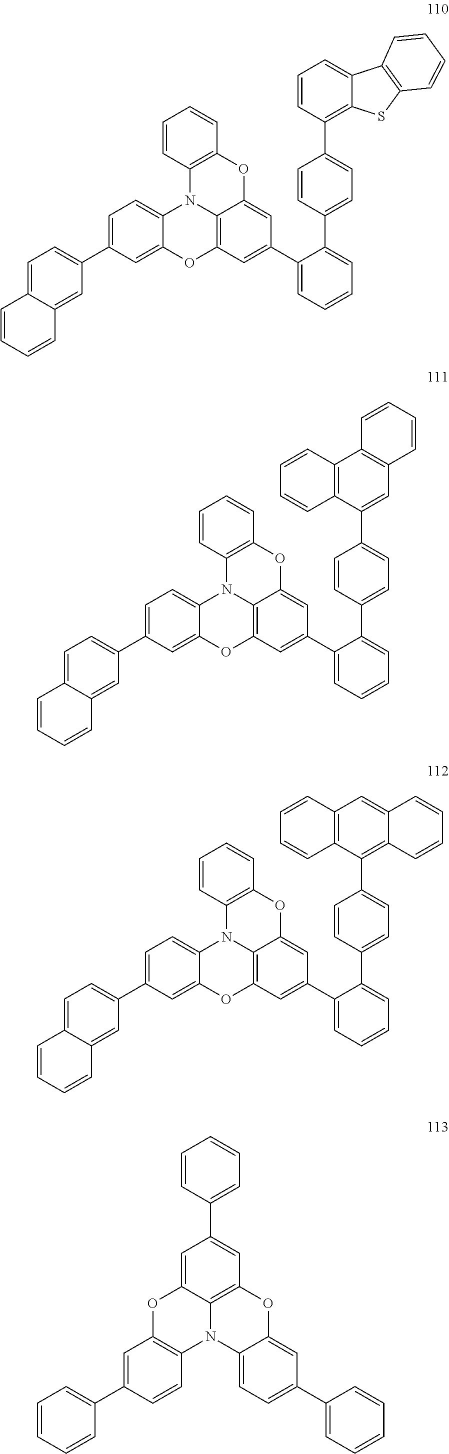 [SMILES: c1ccc2c(c1)Oc1cc(-c3ccccc3-c3ccc(-c4cccc5c4sc4ccccc45)cc3)cc3c1N2c1ccc(-c2ccc4ccccc4c2)cc1O3 ; c1ccc2c(c1)Oc1cc(-c3ccccc3-c3ccc(-c4cc5ccccc5c5ccccc45)cc3)cc3c1N2c1ccc(-c2ccc4ccccc4c2)cc1O3 ; c1ccc2c(c1)Oc1cc(-c3ccccc3-c3ccc(-c4c5ccccc5cc5ccccc45)cc3)cc3c1N2c1ccc(-c2ccc4ccccc4c2)cc1O3 ; c1ccc(-c2ccc3c(c2)Oc2cc(-c4ccccc4)cc4c2N3c2ccc(-c3ccccc3)cc2O4)cc1]

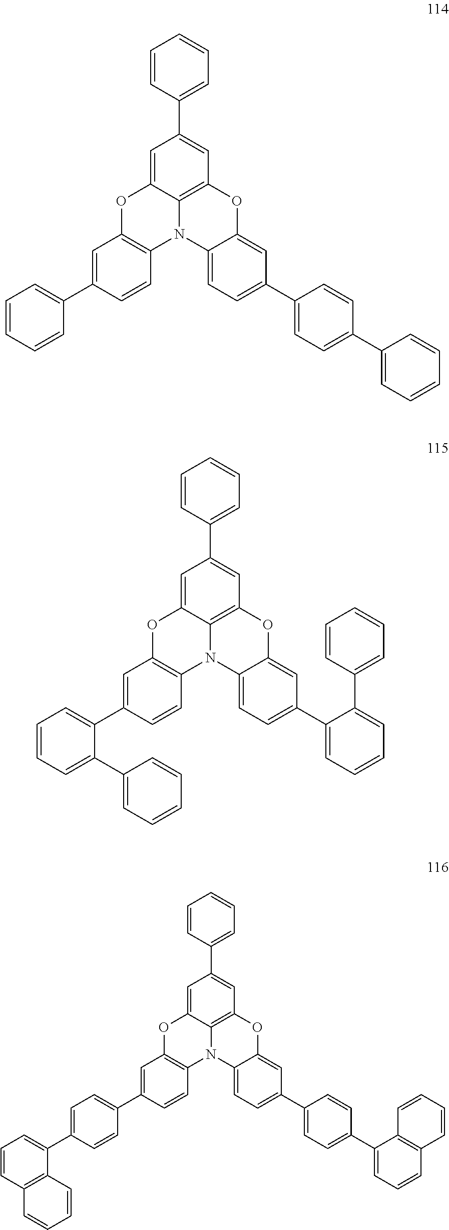 [SMILES: c1ccc(-c2ccc(-c3ccc4c(c3)Oc3cc(-c5ccccc5)cc5c3N4c3ccc(-c4ccccc4)cc3O5)cc2)cc1 ; c1ccc(-c2cc3c4c(c2)Oc2cc(-c5ccccc5-c5ccccc5)ccc2N4c2ccc(-c4ccccc4-c4ccccc4)cc2O3)cc1 ; c1ccc(-c2cc3c4c(c2)Oc2cc(-c5ccc(-c6cccc7ccccc67)cc5)ccc2N4c2ccc(-c4ccc(-c5cccc6ccccc56)cc4)cc2O3)cc1]

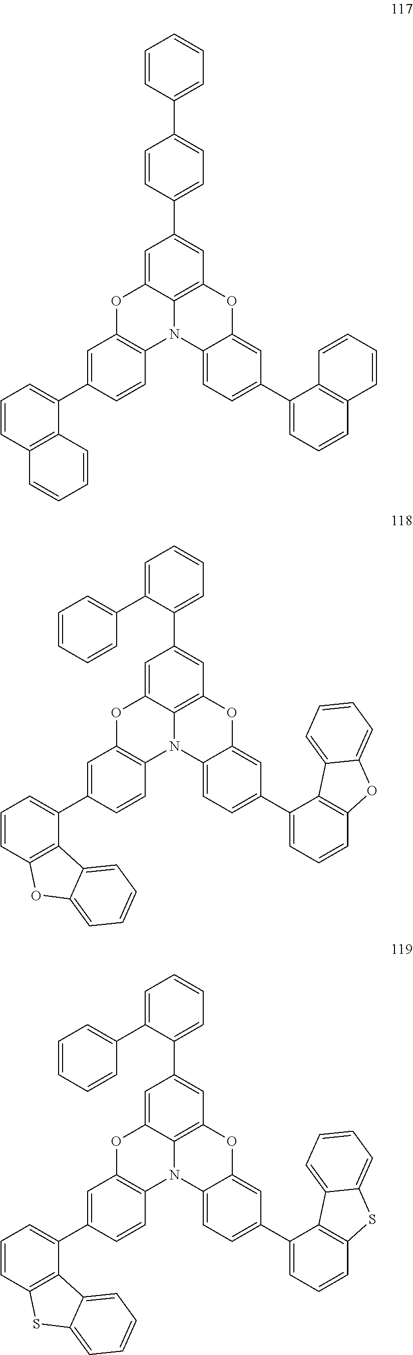 [SMILES: c1ccc(-c2ccc(-c3cc4c5c(c3)Oc3cc(-c6cccc7ccccc67)ccc3N5c3ccc(-c5cccc6ccccc56)cc3O4)cc2)cc1 ; c1ccc(-c2ccccc2-c2cc3c4c(c2)Oc2cc(-c5cccc6oc7ccccc7c56)ccc2N4c2ccc(-c4cccc5oc6ccccc6c45)cc2O3)cc1 ; c1ccc(-c2ccccc2-c2cc3c4c(c2)Oc2cc(-c5cccc6sc7ccccc7c56)ccc2N4c2ccc(-c4cccc5sc6ccccc6c45)cc2O3)cc1]

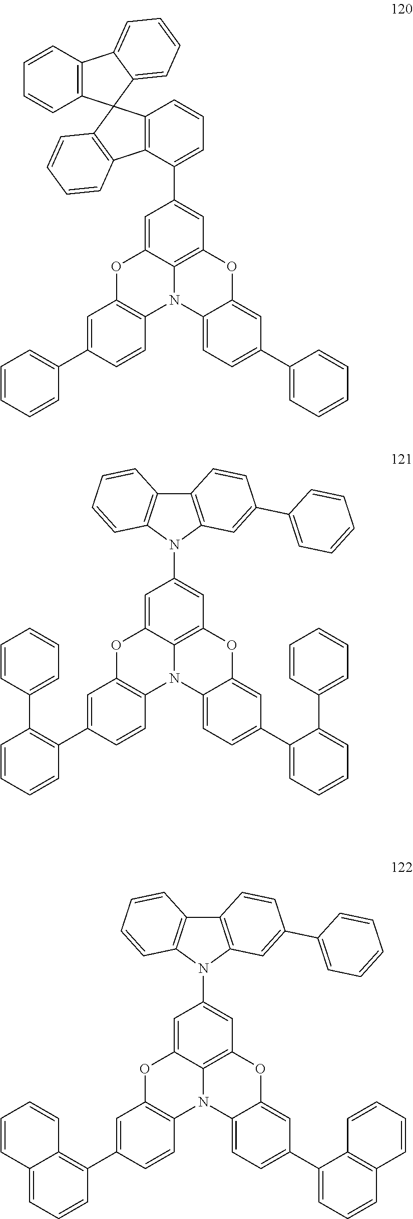 [SMILES: c1ccc(-c2ccc3c(c2)Oc2cc(-c4cccc5c4-c4ccccc4C54c5ccccc5-c5ccccc54)cc4c2N3c2ccc(-c3ccccc3)cc2O4)cc1 ; c1ccc(-c2ccc3c4ccccc4n(-c4cc5c6c(c4)Oc4cc(-c7ccccc7-c7ccccc7)ccc4N6c4ccc(-c6ccccc6-c6ccccc6)cc4O5)c3c2)cc1 ; c1ccc(-c2ccc3c4ccccc4n(-c4cc5c6c(c4)Oc4cc(-c7cccc8ccccc78)ccc4N6c4ccc(-c6cccc7ccccc67)cc4O5)c3c2)cc1]

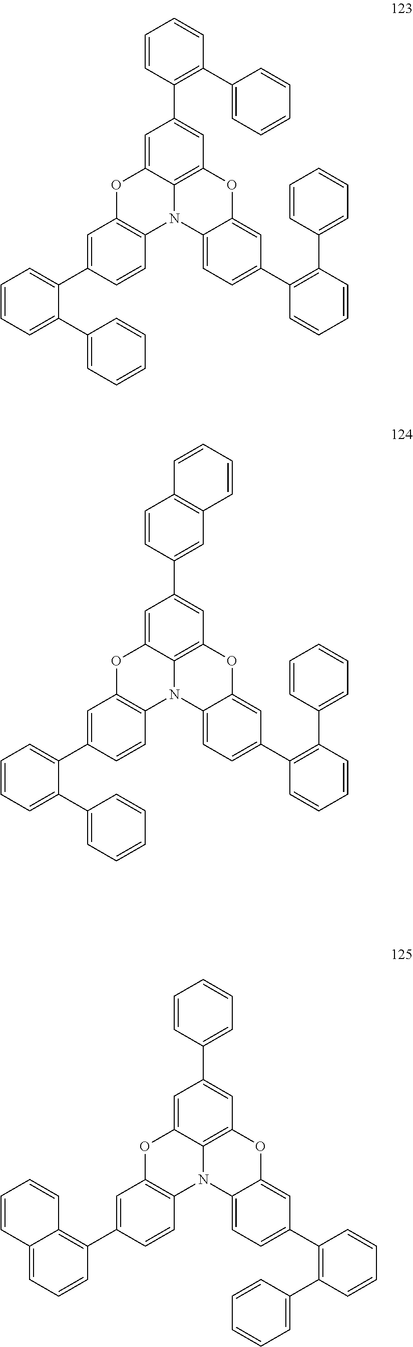 [SMILES: c1ccc(-c2ccccc2-c2ccc3c(c2)Oc2cc(-c4ccccc4-c4ccccc4)cc4c2N3c2ccc(-c3ccccc3-c3ccccc3)cc2O4)cc1 ; c1ccc(-c2ccccc2-c2ccc3c(c2)Oc2cc(-c4ccc5ccccc5c4)cc4c2N3c2ccc(-c3ccccc3-c3ccccc3)cc2O4)cc1 ; c1ccc(-c2cc3c4c(c2)Oc2cc(-c5cccc6ccccc56)ccc2N4c2ccc(-c4ccccc4-c4ccccc4)cc2O3)cc1]

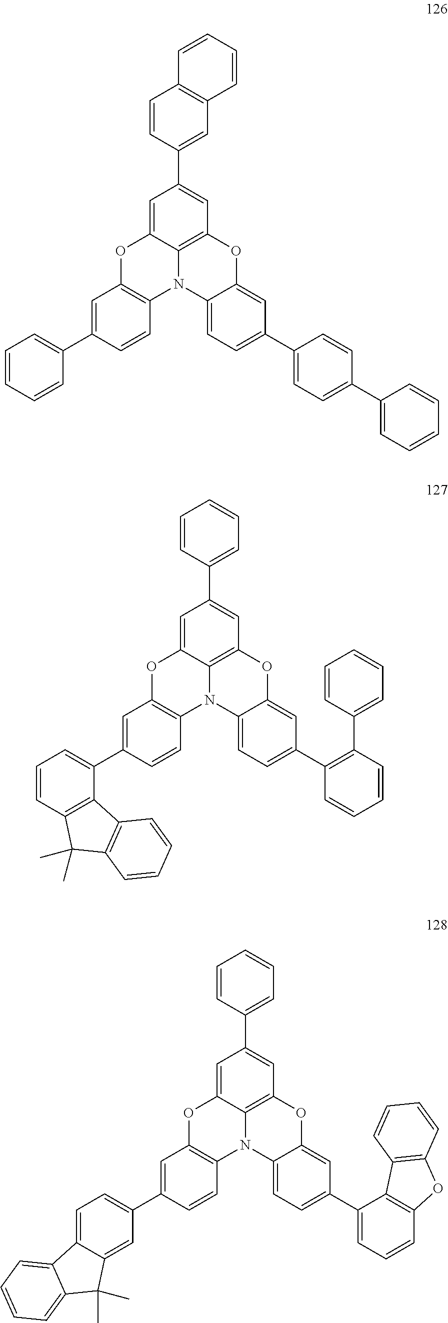 [SMILES: c1ccc(-c2ccc(-c3ccc4c(c3)Oc3cc(-c5ccc6ccccc6c5)cc5c3N4c3ccc(-c4ccccc4)cc3O5)cc2)cc1 ; CC1(C)c2ccccc2-c2c(-c3ccc4c(c3)Oc3cc(-c5ccccc5)cc5c3N4c3ccc(-c4ccccc4-c4ccccc4)cc3O5)cccc21 ; CC1(C)c2ccccc2-c2ccc(-c3ccc4c(c3)Oc3cc(-c5ccccc5)cc5c3N4c3ccc(-c4cccc6oc7ccccc7c46)cc3O5)cc21]

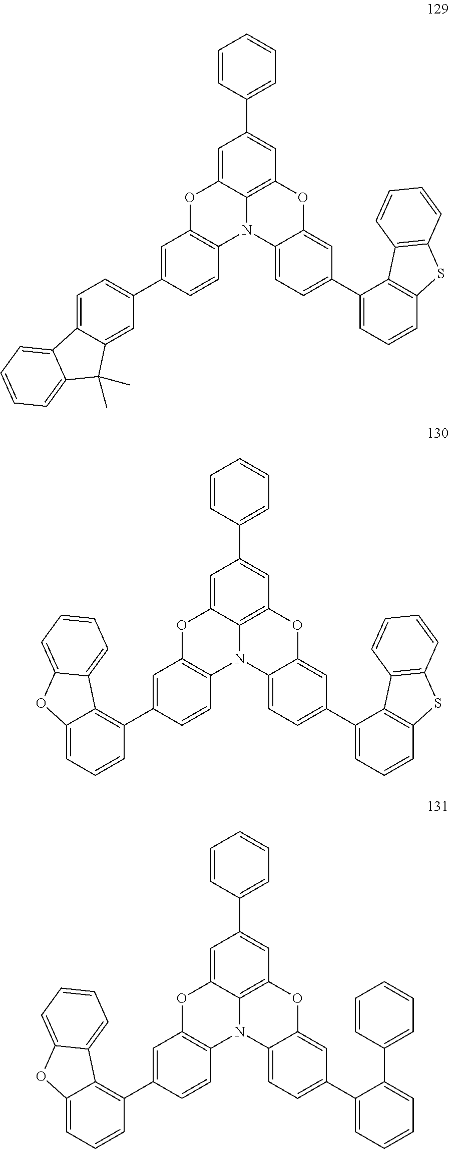 [SMILES: CC1(C)c2ccccc2-c2ccc(-c3ccc4c(c3)Oc3cc(-c5ccccc5)cc5c3N4c3ccc(-c4cccc6sc7ccccc7c46)cc3O5)cc21 ; c1ccc(-c2cc3c4c(c2)Oc2cc(-c5cccc6sc7ccccc7c56)ccc2N4c2ccc(-c4cccc5oc6ccccc6c45)cc2O3)cc1 ; c1ccc(-c2cc3c4c(c2)Oc2cc(-c5cccc6oc7ccccc7c56)ccc2N4c2ccc(-c4ccccc4-c4ccccc4)cc2O3)cc1]

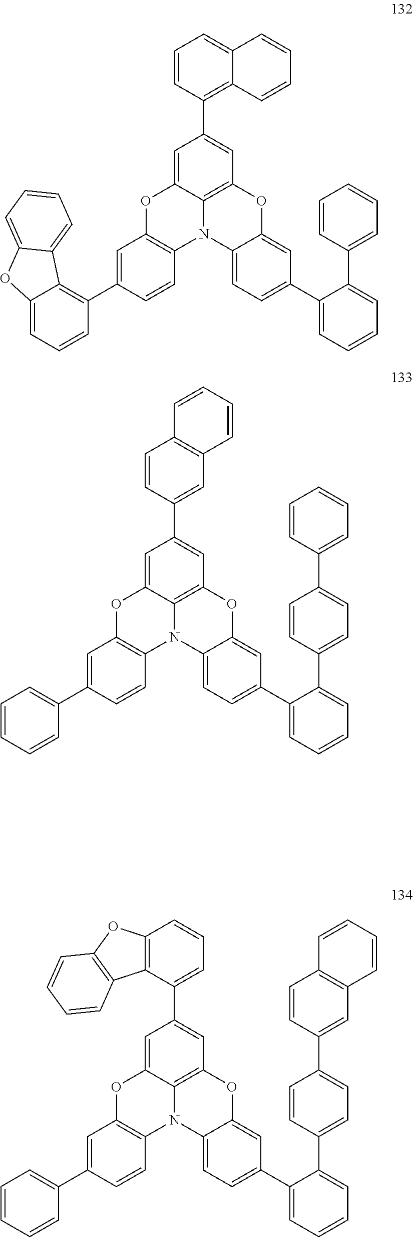 [SMILES: c1ccc(-c2ccccc2-c2ccc3c(c2)Oc2cc(-c4cccc5ccccc45)cc4c2N3c2ccc(-c3cccc5oc6ccccc6c35)cc2O4)cc1 ; c1ccc(-c2ccc(-c3ccccc3-c3ccc4c(c3)Oc3cc(-c5ccc6ccccc6c5)cc5c3N4c3ccc(-c4ccccc4)cc3O5)cc2)cc1 ; c1ccc(-c2ccc3c(c2)Oc2cc(-c4cccc5oc6ccccc6c45)cc4c2N3c2ccc(-c3ccccc3-c3ccc(-c5ccc6ccccc6c5)cc3)cc2O4)cc1]

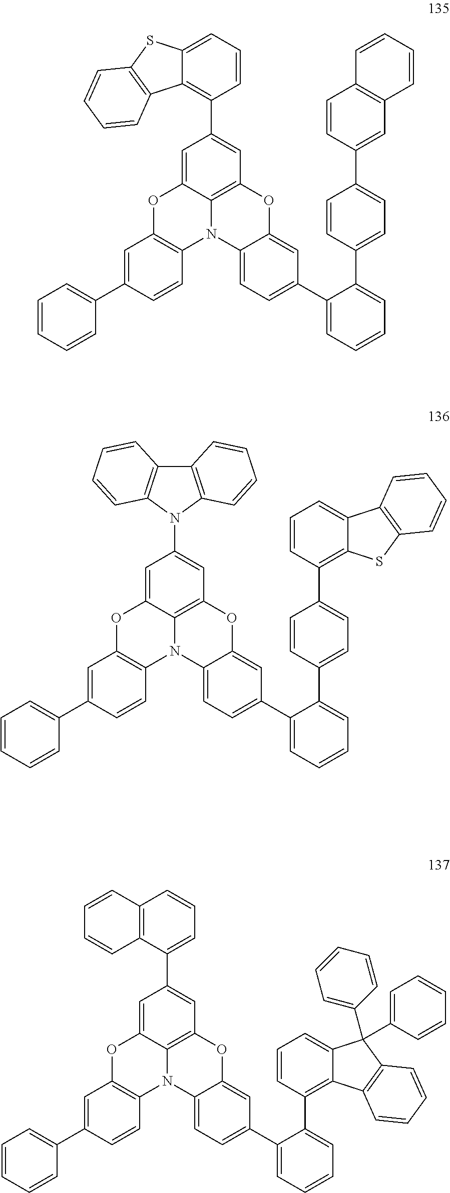 [SMILES: c1ccc(-c2ccc3c(c2)Oc2cc(-c4cccc5sc6ccccc6c45)cc4c2N3c2ccc(-c3ccccc3-c3ccc(-c5ccc6ccccc6c5)cc3)cc2O4)cc1 ; c1ccc(-c2ccc3c(c2)Oc2cc(-n4c5ccccc5c5ccccc54)cc4c2N3c2ccc(-c3ccccc3-c3ccc(-c5cccc6c5sc5ccccc56)cc3)cc2O4)cc1 ; c1ccc(-c2ccc3c(c2)Oc2cc(-c4cccc5ccccc45)cc4c2N3c2ccc(-c3ccccc3-c3cccc5c3-c3ccccc3C5(c3ccccc3)c3ccccc3)cc2O4)cc1]

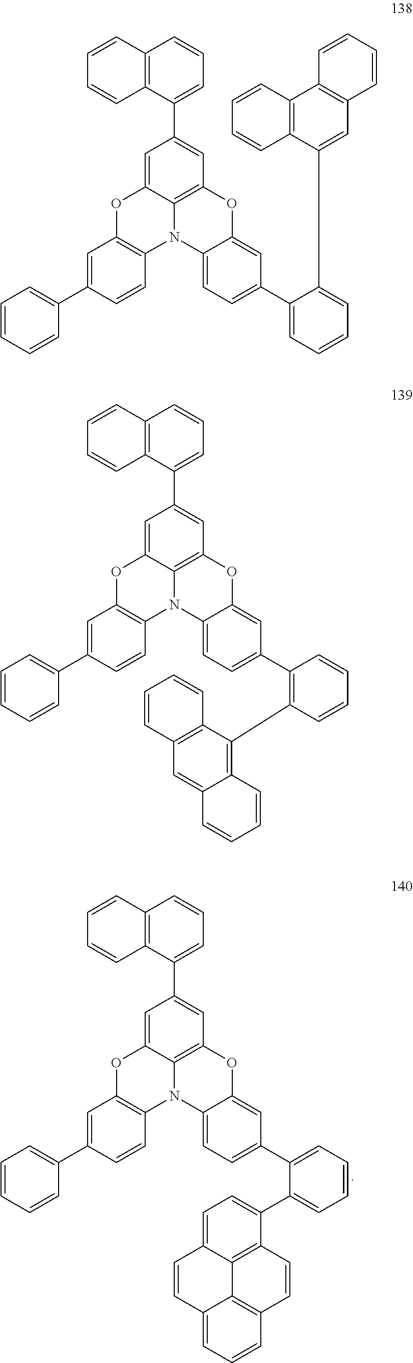 [SMILES: c1ccc(-c2ccc3c(c2)Oc2cc(-c4cccc5ccccc45)cc4c2N3c2ccc(-c3ccccc3-c3cc5ccccc5c5ccccc35)cc2O4)cc1 ; c1ccc(-c2ccc3c(c2)Oc2cc(-c4cccc5ccccc45)cc4c2N3c2ccc(-c3ccccc3-c3c5ccccc5cc5ccccc35)cc2O4)cc1 ; c1ccc(-c2ccc3c(c2)Oc2cc(-c4cccc5ccccc45)cc4c2N3c2ccc(-c3ccccc3-c3ccc5ccc6cccc7ccc3c5c67)cc2O4)cc1]

10. The light-emitting device of claim 1, wherein the first electrode comprises a first layer, a second layer, and a third layer between the first layer and the interlayer,
the second layer is located between the first layer and the third layer,
the first layer comprises a first material,
the second layer comprises a second material,
the third layer comprises the inorganic material,
the first material and the second material are different from each other, and
the second material and the inorganic material are different from each other.

11. The light-emitting device of claim 10, wherein the first layer is in direct contact with the second layer.

12. The light-emitting device of claim 10, wherein the second layer is in direct contact with the third layer.

13. The light-emitting device of claim 10, wherein the third layer is in direct contact with the hole transport layer.

14. The light-emitting device of claim 10, wherein the first material comprises a conductive oxide material.

15. The light-emitting device of claim 10, wherein the second material comprises a metal material or a metal alloy material.

16. The light-emitting device of claim 10, wherein the first material and the inorganic material are different from each other.

17. The light-emitting device of claim 10, wherein the third layer consists of the inorganic material.

18. An electronic apparatus comprising the light-emitting device of claim 1.

19. The electronic apparatus of claim 18, further comprising:
a thin-film transistor comprising a source electrode, a drain electrode, and an activation layer, wherein
the first electrode of the light-emitting device is electrically connected with the source electrode or the drain electrode of the thin-film transistor.

20. The electronic apparatus of claim 18, further comprising:
a touch screen layer, a polarizing layer, a color filter, a color conversion layer, or any combination thereof.
